SEQUENCE LISTING (128 sequences)

SEQ ID NO: 1 - protein, 339 aa, synthetic construct
MQIITTALVCLLLAGMWPEDVDSKSMQVPFSRCCFSFAEQEIPLRAILCYRNTSSICSNEGLIFKLKRGKEACALDTVGWVQRHRKMLRHCPSKRKSGGSGGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGEPEA

SEQ ID NO: 2 - protein, 96 aa, Homo sapiens
MQIITTALVCLLLAGMWPEDVDSKSMQVPFSRCCFSFAEQEIPLRAILCYRNTSSICSNEGLIFKLKRGKEACALDTVGWVQRHRKMLRHCPSKRK

SEQ ID NO: 3 - protein, 9 aa, synthetic construct
SGGSGGGSG

SEQ ID NO: 4 - protein, 234 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGEPEA

SEQ ID NO: 5 - protein, 322 aa, synthetic construct
LARRKCCLNPTNRPIPNPLLQDLSRVDYQAIGHDCGREAFRVTLQDGRQGCVSVGNKSLLDWLRGHKDLCPQIWSGCESLGGGSGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGEPEA

SEQ ID NO: 6 - protein, 80 aa, synthetic construct
LARRKCCLNPTNRPIPNPLLQDLSRVDYQAIGHDCGREAFRVTLQDGRQGCVSVGNKSLLDWLRGHKDLCPQIWSGCESL

SEQ ID NO: 7 - protein, 8 aa, synthetic construct
GGGSGGGS

SEQ ID NO: 8 - protein, 123 aa, synthetic construct
EVQLVETGGGLVQPKGSLKLSCAASGFTFNINAMNWVRQAPGKGLEWVARIRSKSNNYATYYADSVKDRFTISRDDSQSMLYLQMNNLKTEDTAMYYCVRGGYGSSPYDMDYWGQGTSVTVSS

SEQ ID NO: 9 - protein, 112 aa, synthetic construct
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGAGTKLELK

SEQ ID NO: 10 - protein, 446 aa, synthetic construct
EVQLVETGGGLVQPKGSLKLSCAASGFTFNINAMNWVRQAPGKGLEWVARIRSKSNNYATYYADSVKDRFTISRDDSQSMLYLQMNNLKTEDTAMYYCVRGGYGSSPYDMDYWGQGTSVTVSSAKTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPG

SEQ ID NO: 11 - protein, 219 aa, synthetic construct
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 10 aa, synthetic construct
GFTFNINAMN

SEQ ID NO: 13 - protein, 17 aa, synthetic construct
RIRSKSNNYATYYADSV

SEQ ID NO: 14 - protein, 14 aa, synthetic construct
VRGGYGSSPYDMDY

SEQ ID NO: 15 - protein, 16 aa, synthetic construct
RSSKSLLHSNGNTYLY

SEQ ID NO: 16 - protein, 7 aa, synthetic construct
RMSNLAS

SEQ ID NO: 17 - protein, 9 aa, synthetic construct
MQHLEYPFT

SEQ ID NO: 18 - protein, 452 aa, synthetic construct
EVQLVETGGGLVQPKGSLKLSCAASGFTFNINAMNWVRQAPGKGLEWVARIRSKSNNYATYYADSVKDRFTISRDDSQSMLYLQMNNLKTEDTAMYYCVRGGYGSSPYDMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 219 aa, synthetic construct
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 115 aa, synthetic construct
QVQLKESGPGLVAPSQSLSITCTVSGFSLARYDISWIRQPPGKGLEWLGVIWTGGGTNYNSAFMSRLSISKDNSKSQVFLKMNGLQTDDTAIYYCVSIRYDETYWGQGTLVTVSA

SEQ ID NO: 21 - protein, 106 aa, synthetic construct
QIVLTQSPAIMSASPGEKVTITCSASSSVIYMHWFQQKPGTSPKLWIYATSNLASGVPARFSGSGSGTSYSLTISRMEAEDAATYYCQQRSSYPLTFGAGTKLELK

SEQ ID NO: 22 - protein, 444 aa, synthetic construct
QVQLKESGPGLVAPSQSLSITCTVSGFSLARYDISWIRQPPGKGLEWLGVIWTGGGTNYNSAFMSRLSISKDNSKSQVFLKMNGLQTDDTAIYYCVSIRYDETYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 23 - protein, 213 aa, synthetic construct
QIVLTQSPAIMSASPGEKVTITCSASSSVIYMHWFQQKPGTSPKLWIYATSNLASGVPARFSGSGSGTSYSLTISRMEAEDAATYYCQQRSSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 24 - protein, 10 aa, synthetic construct
GFSLARYDIS

SEQ ID NO: 25 - protein, 16 aa, synthetic construct
GGTNYNSAFMSRLSIS

SEQ ID NO: 26 - protein, 9 aa, synthetic construct
VSIRYDETY

SEQ ID NO: 27 - protein, 10 aa, synthetic construct
SASSSVIYMH

SEQ ID NO: 28 - protein, 7 aa, synthetic construct
TSNLASG

SEQ ID NO: 29 - protein, 9 aa, synthetic construct
QQRSSYPLT

SEQ ID NO: 30 - protein, 444 aa, synthetic construct
QVQLKESGPGLVAPSQSLSITCTVSGFSLARYDISWIRQPPGKGLEWLGVIWTGGGTNYNSAFMSRLSISKDNSKSQVFLKMNGLQTDDTAIYYCVSIRYDETYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 213 aa, synthetic construct
QIVLTQSPAIMSASPGEKVTITCSASSSVIYMHWFQQKPGTSPKLWIYATSNLASGVPARFSGSGSGTSYSLTISRMEAEDAATYYCQQRSSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 128 aa, synthetic construct
EVQLVETGGGLVQPKGSLKLSCAASGFTFSTNAMNWVRQAPGKGLEWIARIRSKSNNYATHYADSVKDRFTISRDDSQSILHLQMNNLKNEDTAMYYCVRDSHYYVSTYVGLAWFAYWGQGTLVTVSA

SEQ ID NO: 33 - protein, 106 aa, synthetic construct
QIVLTQSPAIMSASPGEKVTITCSASSSVSYMHWFQQKPGTSPKLWIYSTSNLASGVPARFSGSGSGTSYFLTISRMEAEDAATYYCQQRSSYPYTFGGGTKLERK

SEQ ID NO: 34 - protein, 457 aa, synthetic construct
EVQLVETGGGLVQPKGSLKLSCAASGFTFSTNAMNWVRQAPGKGLEWIARIRSKSNNYATHYADSVKDRFTISRDDSQSILHLQMNNLKNEDTAMYYCVRDSHYYVSTYVGLAWFAYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 35 - protein, 213 aa, synthetic construct
QIVLTQSPAIMSASPGEKVTITCSASSSVSYMHWFQQKPGTSPKLWIYSTSNLASGVPARFSGSGSGTSYFLTISRMEAEDAATYYCQQRSSYPYTFGGGTKLERKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 36 - protein, 10 aa, synthetic construct
GFTFSTNAMN

SEQ ID NO: 37 - protein, 17 aa, synthetic construct
RIRSKSNNYATHYADSV

SEQ ID NO: 38 - protein, 15 aa, synthetic construct
VRDSHYYVSTYVGLA

SEQ ID NO: 39 - protein, 10 aa, synthetic construct
SASSSVSYMH

SEQ ID NO: 40 - protein, 6 aa, synthetic construct
TSNLAS

SEQ ID NO: 41 - protein, 9 aa, synthetic construct
QQRSSYPYT

SEQ ID NO: 42 - protein, 457 aa, synthetic construct
EVQLVETGGGLVQPKGSLKLSCAASGFTFSTNAMNWVRQAPGKGLEWIARIRSKSNNYATHYADSVKDRFTISRDDSQSILHLQMNNLKNEDTAMYYCVRDSHYYVSTYVGLAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 213 aa, synthetic construct
QIVLTQSPAIMSASPGEKVTITCSASSSVSYMHWFQQKPGTSPKLWIYSTSNLASGVPARFSGSGSGTSYFLTISRMEAEDAATYYCQQRSSYPYTFGGGTKLERKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 123 aa, synthetic construct
EVQLQQSVAELVRPGASVKLSCTASGFNIKNTQMHWVKQRPEQGLEWIGRIDPANGNTKYAPKFQGKATITGDTSSNTAYLQLSSLTSEDTAIYYCARFDYYYGSGDYAMDYWGQGTSVTVSS

SEQ ID NO: 45 - protein, 112 aa, synthetic construct
DVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTRFPWTFGGGTNLEIK

SEQ ID NO: 46 - protein, 521 aa, synthetic construct
EVQLQQSVAELVRPGASVKLSCTASGFNIKNTQMHWVKQRPEQGLEWIGRIDPANGNTKYAPKFQGKATITGDTSSNTAYLQLSSLTSEDTAIYYCARFDYYYGSGDYAMDYWGQGTSVTVSSTTTAPSVYPLVPGCSDTSGSSVTLGCLVKGYFPEPVTVKWNYGALSSGVRTVSSVLQSGFYSLSSLVTVPSSTWPSQTVICNVAHPASKTELIKRIEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPELELNETCAEAQDGELDGLWTTITIFISLFLLSVCYSASVTLFKVKWIFSSVVQVKQTAIPDYRNMIGQGA

SEQ ID NO: 47 - protein, 219 aa, synthetic construct
DVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTRFPWTFGGGTNLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 48 - protein, 10 aa, synthetic construct
GFNIKNTQMH

SEQ ID NO: 49 - protein, 13 aa, synthetic construct
RIDPANGNTKYAP

SEQ ID NO: 50 - protein, 16 aa, synthetic construct
ARFDYYYGSGDYAMDY

SEQ ID NO: 51 - protein, 16 aa, synthetic construct
KSSQSLLYSNGKTYLN

SEQ ID NO: 52 - protein, 7 aa, synthetic construct
LVSKLDS

SEQ ID NO: 53 - protein, 9 aa, synthetic construct
VQGTRFPWT

SEQ ID NO: 54 - protein, 452 aa, synthetic construct
EVQLQQSVAELVRPGASVKLSCTASGFNIKNTQMHWVKQRPEQGLEWIGRIDPANGNTKYAPKFQGKATITGDTSSNTAYLQLSSLTSEDTAIYYCARFDYYYGSGDYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 219 aa, synthetic construct
DVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTRFPWTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 117 aa, synthetic construct
QVQLQQPGAELVKPGASVKMSCKASGYTFTSYNMHWVKQTPGQGLEWIGAIYPGNGDTSYNQKFKGKATLTADKSSSTAYMHLSSLTSEDSAVYYCARKGGTPFAYWGQGTLLTVSA

SEQ ID NO: 57 - protein, 112 aa, synthetic construct
DVVMTQTPLSLPVSLGDQASISCRSSQSLVHSNGNTYLYWYLQKPGQSPKLLIYKVSNRFSGVPDRFSGSGSGTDFTLKISRVEAEDLGVYFCSQSTHVPYTFGGGTKLEIK

SEQ ID NO: 58 - protein, 440 aa, synthetic construct
QVQLQQPGAELVKPGASVKMSCKASGYTFTSYNMHWVKQTPGQGLEWIGAIYPGNGDTSYNQKFKGKATLTADKSSSTAYMHLSSLTSEDSAVYYCARKGGTPFAYWGQGTLLTVSAAKTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPG

SEQ ID NO: 59 - protein, 219 aa, synthetic construct
DVVMTQTPLSLPVSLGDQASISCRSSQSLVHSNGNTYLYWYLQKPGQSPKLLIYKVSNRFSGVPDRFSGSGSGTDFTLKISRVEAEDLGVYFCSQSTHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 60 - protein, 10 aa, synthetic construct
GYTFTSYNMH

SEQ ID NO: 61 - protein, 13 aa, synthetic construct
AIYPGNGDTSYNQ

SEQ ID NO: 62 - protein, 10 aa, synthetic construct
ARKGGTPFAY

SEQ ID NO: 63 - protein, 16 aa, synthetic construct
RSSQSLVHSNGNTYLY

SEQ ID NO: 64 - protein, 7 aa, synthetic construct
KVSNRFS

SEQ ID NO: 65 - protein, 9 aa, synthetic construct
SQSTHVPYT

SEQ ID NO: 66 - protein, 446 aa, synthetic construct
QVQLQQPGAELVKPGASVKMSCKASGYTFTSYNMHWVKQTPGQGLEWIGAIYPGNGDTSYNQKFKGKATLTADKSSSTAYMHLSSLTSEDSAVYYCARKGGTPFAYWGQGTLLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 219 aa, synthetic construct
DVVMTQTPLSLPVSLGDQASISCRSSQSLVHSNGNTYLYWYLQKPGQSPKLLIYKVSNRFSGVPDRFSGSGSGTDFTLKISRVEAEDLGVYFCSQSTHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 117 aa, synthetic construct
EVQLVQSGAEVKKPGASVKVSCKASGYTFTSYNMHWVRQAPGQRLEWIGAIYPGQGDTSYNQKFKGRATLTADKSASTAYMELSSLRSEDTAVYYCARKGGTPFAYWGQGTLVTVSS

SEQ ID NO: 69 - protein, 112 aa, synthetic construct
DVVMTQSPLSLPVTLGQPASISCRSSQSLVHSNAQTYLYWYQQRPGQSPRLLIYKVSNRFSGVPDRFSGSGSGTDFTLKISRVEAEDVGVYFCSQSTHVPYTFGQGTKLEIK

SEQ ID NO: 70 - protein, 446 aa, synthetic construct
EVQLVQSGAEVKKPGASVKVSCKASGYTFTSYNMHWVRQAPGQRLEWIGAIYPGQGDTSYNQKFKGRATLTADKSASTAYMELSSLRSEDTAVYYCARKGGTPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 219 aa, synthetic construct
DVVMTQSPLSLPVTLGQPASISCRSSQSLVHSNAQTYLYWYQQRPGQSPRLLIYKVSNRFSGVPDRFSGSGSGTDFTLKISRVEAEDVGVYFCSQSTHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 13 aa, synthetic construct
AIYPGQGDTSYNQ

SEQ ID NO: 73 - protein, 16 aa, synthetic construct
RSSQSLVHSNAQTYLY

SEQ ID NO: 74 - protein, 117 aa, synthetic construct
EVQLVQSGAEVKKPGASVKVSCKASGYTFTSYNMHWVRQAPGQGLEWIGAIYPGAGDTSYNQKFKGRATLTADKSTSTAYMELSSLRSEDTAVYYCARKGGTPFAYWGQGTLVTVSS

SEQ ID NO: 75 - protein, 112 aa, synthetic construct
DVVMTQSPLSLPVTLGQPASISCRSSQSLVHSNAATYLYWYQQRPGQSPRLLIYKVSNRFSGVPDRFSGSGSGTDFTLKISRVEAEDVGVYFCSQSTHVPYTFGQGTKLEIK

SEQ ID NO: 76 - protein, 446 aa, synthetic construct
EVQLVQSGAEVKKPGASVKVSCKASGYTFTSYNMHWVRQAPGQGLEWIGAIYPGAGDTSYNQKFKGRATLTADKSTSTAYMELSSLRSEDTAVYYCARKGGTPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 219 aa, synthetic construct
DVVMTQSPLSLPVTLGQPASISCRSSQSLVHSNAATYLYWYQQRPGQSPRLLIYKVSNRFSGVPDRFSGSGSGTDFTLKISRVEAEDVGVYFCSQSTHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 13 aa, synthetic construct
AIYPGAGDTSYNQ

SEQ ID NO: 79 - protein, 16 aa, synthetic construct
RSSQSLVHSNAATYLY

SEQ ID NO: 80 - protein, 121 aa, synthetic construct
EVQLVESGGGLVQPKGSLKLSCAASGFTFNTYAMNWVRQAPGKGLEWVARIRSKSNNYATYYADSVKDRFTISRDDSQSMLYLQMNNLKTEDTAMYYCVRGLLRYRFFDVWGAGTTVTVSS

SEQ ID NO: 81 - protein, 112 aa, synthetic construct
DIVMTQAEPSVPVTPGESISISCRSSKSLLHSNGNTYLYWFLQRPGQSPQLLIYRMSNLASGVPDRFSGSGSGTAFTLRISRVEAEDVGVYYCMQHLEYPFTFGGGTKLEIK

SEQ ID NO: 82 - protein, 444 aa, synthetic construct
EVQLVESGGGLVQPKGSLKLSCAASGFTFNTYAMNWVRQAPGKGLEWVARIRSKSNNYATYYADSVKDRFTISRDDSQSMLYLQMNNLKTEDTAMYYCVRGLLRYRFFDVWGAGTTVTVSSAKTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPG

SEQ ID NO: 83 - protein, 219 aa, synthetic construct
DIVMTQAEPSVPVTPGESISISCRSSKSLLHSNGNTYLYWFLQRPGQSPQLLIYRMSNLASGVPDRFSGSGSGTAFTLRISRVEAEDVGVYYCMQHLEYPFTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 84 - protein, 10 aa, synthetic construct
GFTFNTYAMN

SEQ ID NO: 85 - protein, 19 aa, synthetic construct
RIRSKSNNYATYYADSVKD

SEQ ID NO: 86 - protein, 12 aa, synthetic construct
VRGLLRYRFFDV

SEQ ID NO: 87 - protein, 16 aa, synthetic construct
RSSKSLLHSNGNTYLY

SEQ ID NO: 88 - protein, 7 aa, synthetic construct
RMSNLAS

SEQ ID NO: 89 - protein, 9 aa, synthetic construct
MQHLEYPFT

SEQ ID NO: 90 - protein, 450 aa, synthetic construct
EVQLVESGGGLVQPKGSLKLSCAASGFTFNTYAMNWVRQAPGKGLEWVARIRSKSNNYATYYADSVKDRFTISRDDSQSMLYLQMNNLKTEDTAMYYCVRGLLRYRFFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 219 aa, synthetic construct
DIVMTQAEPSVPVTPGESISISCRSSKSLLHSNGNTYLYWFLQRPGQSPQLLIYRMSNLASGVPDRFSGSGSGTAFTLRISRVEAEDVGVYYCMQHLEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 121 aa, synthetic construct
EVQLVESGGGLVQPGGSLKLSCAASGFTFNTYAMNWVRQASGKGLEWVGRIRSKSNNYATYYADSVKDRFTISRDDSKNTAYLQMNSLKTEDTAVYYCVRGLLRYRFFDVWGQGTTVTVSS

SEQ ID NO: 93 - protein, 112 aa, synthetic construct
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCMQHLEYPFTFGGGTKVEIK

SEQ ID NO: 94 - protein, 450 aa, synthetic construct
EVQLVESGGGLVQPGGSLKLSCAASGFTFNTYAMNWVRQASGKGLEWVGRIRSKSNNYATYYADSVKDRFTISRDDSKNTAYLQMNSLKTEDTAVYYCVRGLLRYRFFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 219 aa, synthetic construct
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCMQHLEYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 121 aa, synthetic construct
EVQLVESGGGLVQPGRSLRLSCTASGFTFATYAMNWVRQAPGKGLEWVARIRSKSNNYATYYADSVKDRFTISRDDSKSILYLQMNSLKTEDTAVYYCVRGLLRYRFFDVWGQGTTVTVSS

SEQ ID NO: 97 - protein, 112 aa, synthetic construct; identical to SEQ ID NO: 93

SEQ ID NO: 98 - protein, 450 aa, synthetic construct
EVQLVESGGGLVQPGRSLRLSCTASGFTFATYAMNWVRQAPGKGLEWVARIRSKSNNYATYYADSVKDRFTISRDDSKSILYLQMNSLKTEDTAVYYCVRGLLRYRFFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 219 aa, synthetic construct; identical to SEQ ID NO: 95

SEQ ID NO: 100 - protein, 10 aa, synthetic construct
GFTFATYAMN

SEQ ID NO: 101 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 102 - protein, 353 aa, Mus sp.
MDYTMEPNVTMTDYYPDFFTAPCDAEFLLRGSMLYLAILYCVLFVLGLLGNSLVILVLVGCKKLRSITDIYLLNLAASDLLFVLSIPFQTHNLLDQWVFGTAMCKVVSGLYYIGFFSSMFFITLMSVDRYLAIVHAVYAIKVRTASVGTALSLTVWLAAVTATIPLMVFYQVASEDGMLQCFQFYEEQSLRWKLFTHFEINALGLLLPFAILLFCYVRILQQLRGCLNHNRTRAIKLVLTVVIVSLLFWVPFNVALFLTSLHDLHILDGCATRQRLALAIHVTEVISFTHCCVNPVIYAFIGEKFKKHLMDVFQKSCSHIFLYLGRQMPVGALERQLSSNQRSSHSSTLDDIL

SEQ ID NO: 103 - protein, 355 aa, Macaca fascicularis
MDYTLDPSMTTMTDYYYPDSLSSPCDGELIQRNDKLLLAVFYCLLFVFSLLGNSLVILVLVVCKKLRNITDIYLLNLALSDLLFVFSFPFQTYYQLDQWVFGTVMCKVVSGFYYIGFYSSMFFITLMSVDRYLAVVHAVYAIKVRTIRMGTTLSLVVWLTAIMATIPLLVFYQVASEDGVLQCYSFYNQQTLKWKIFTNFEMNILGLLIPFTIFMFCYIKILHQLKRCQNHNKTKAIRLVLIVVIASLLFWVPFNVVLFLTSLHSMHILDGCSISQQLNYATHVTEIISFTHCCVNPVIYAFVGEKFKKHLSEIFQKSCSHIFIYLGRQMPRESCEKSSSCQQHSFRSSSIDYIL

SEQ ID NO: 104 - protein, 12 aa, synthetic construct
ARGLLRYRFFDV

SEQ ID NO: 105 - protein, 12 aa, synthetic construct
VAGLLRYRFFDV

SEQ ID NO: 106 - protein, 12 aa, synthetic construct
VRALLRYRFFDV

SEQ ID NO: 107 - protein, 12 aa, synthetic construct
VRGALRYRFFDV

SEQ ID NO: 108 - protein, 12 aa, synthetic construct
VRGLARYRFFDV

SEQ ID NO: 109 - protein, 12 aa, synthetic construct
VRGLLAYRFFDV

SEQ ID NO: 110 - protein, 12 aa, synthetic construct
VRGLLRARFFDV

SEQ ID NO: 111 - protein, 12 aa, synthetic construct
VRGLLRYAFFDV

SEQ ID NO: 112 - protein, 12 aa, synthetic construct
VRGLLRYRAFDV

SEQ ID NO: 113 - protein, 12 aa, synthetic construct
VRGLLRYRFADV

SEQ ID NO: 114 - protein, 12 aa, synthetic construct
VRGLLRYRFFAV

SEQ ID NO: 115 - protein, 12 aa, synthetic construct
VRGLLRYRFFDA

SEQ ID NO: 116 - protein, 12 aa, synthetic construct
ARGLLRARFFDV

SEQ ID NO: 117 - protein, 12 aa, synthetic construct
ARGLLRYRFFDA

SEQ ID NO: 118 - protein, 12 aa, synthetic construct
VRGLLRARFFDA

SEQ ID NO: 119 - protein, 12 aa, synthetic construct
ARGLLRARFFDA

SEQ ID NO: 120 - protein, 16 aa, synthetic construct
RSSQSLLHSNGYNYLD

SEQ ID NO: 121 - protein, 7 aa, synthetic construct
LGSNRAS

SEQ ID NO: 122 - protein, 9 aa, synthetic construct
MQALQTPFV

SEQ ID NO: 123 - protein, 10 aa, synthetic construct
GFTFSGSAMH

SEQ ID NO: 124 - protein, 19 aa, synthetic construct
RIRSKANSYATAYAASVKD

SEQ ID NO: 125 - protein, 11 aa, synthetic construct
TRYYYYYGMDV

SEQ ID NO: 126 - protein, 15 aa, synthetic construct
RRCPLYISYDPVCRR

SEQ ID NO: 127 - protein, 19 aa, synthetic construct
RRRRCPLYISYDPVCRRRR

SEQ ID NO: 128 - protein, 440 aa, synthetic construct
EEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLIDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTSGGGSGGGSGGGSGGGSHHHHHHGGGSGGGGSGGENLYFQGMDYTLDLSVTTVTDYYYPDIFSSPCDAELIQTNGK

(19) United States
(12) Patent Application Publication
(10) Pub. No.: US 2024/0165227 A1
(43) Pub. Date: May 23, 2024

(54) ANTICANCER THERAPIES USING ANTI-CCR8 ANTIBODY, CHEMO AND IMMUNOTHERAPY COMBINATIONS

(71) Applicant: Gilead Sciences, Inc., Foster City, CA (US)

(72) Inventors: Tianling Chen, Mountain View, CA (US); Renu Jain, Redwood City, CA (US); Michelle R. Kuhne, San Francisco, CA (US); Dorothée Saddier Axe, San Carlos, CA (US); Brian M. Weist, Dublin, CA (US)

(21) Appl. No.: 18/499,540

(22) Filed: Nov. 1, 2023

Related U.S. Application Data
(60) Provisional application No. 63/382,386, filed on Nov. 4, 2022.

(51) Int. Cl. A61K 39/395; A61K 31/282; A61K 31/337; A61K 31/4745; A61K 31/513; A61K 31/519; A61K 31/704; A61K 31/7048; A61K 31/7068; A61K 33/243; A61K 39/00; A61P 35/00

(52) U.S. Cl. CPC A61K 39/3955; A61K 31/282; A61K 31/337; A61K 31/4745; A61K 31/513; A61K 31/519; A61K 31/704; A61K 31/7048; A61K 31/7068; A61K 33/243; A61P 35/00; A61K 2039/505

(57) ABSTRACT

The present disclosure relates to methods of treating cancer in a subject by administrating an effective amount of an anti-CCR8 antibody, a chemotherapeutic agent (e.g., cisplatin, gemcitabine, docetaxel), and a PD1 inhibitor or PD-L1 inhibitor to the subject. In some embodiments, the chemotherapeutic agent is administered at a lower dose than in a standard of care chemotherapeutic regimen that does not comprise an anti-CCR8 antibody.

### CROSS REFERENCES TO RELATED APPLICATIONS

This application claims the benefit under 35 U.S.C. § 119(e) of U.S. provisional application No. 63/382,386, filed on Nov. 4, 2022, which is hereby incorporated by reference in its entirety for all purposes.

### FIELD

The present disclosure relates to methods of treating cancer in a subject by co-administrating an effective amount of an anti-CCR8 antibody, a chemotherapeutic agent (e.g., cisplatin, gemcitabine, docetaxel), and a PD1 inhibitor or PD-L1 inhibitor to the subject. In some embodiments, the chemotherapeutic agent is co-administered at a lower dose than in a standard of care chemotherapeutic regimen that does not comprise an anti-CCR8 antibody.

### SEQUENCE LISTING

The instant application contains a Sequence Listing which has been submitted electronically in XML format and is hereby incorporated by reference in its entirety. Said XML copy, created on Oct. 12, 2023, is named 1456-US—NP_SL.xml and is 126,949 bytes in size.

### BACKGROUND

Lack of effector T cell activity in solid tumors can be attributed to suppressive mechanisms utilized by regulatory T cells (Tregs) within the tumor microenvironment. An outstanding question in the field is how to selectively deplete intra-tumoral Tregs to avoid severe autoimmunity triggered by systemic depletion.

Chemokine (C—C motif) receptor 8 (CCR8) belongs to the G protein-coupled receptor (GPCR) family. CCR8 is expressed at high levels on the surface of tumor-infiltrating Tregs, but not on peripheral Tregs, nor effector T cells. CCR8 targeting antibodies, which lead to rapid depletion of intra-tumoral Tregs in mouse models and human explant systems, are currently in the clinic as monotherapy and in combination with checkpoint inhibitors.

There remains a need for further combination therapies to improve the efficacy of anti-CCR8 antibody based anticancer treatments.

### SUMMARY

In one aspect, provided herein is a method of treating cancer in a subject comprising co-administering to the subject an effective amount of (i) an anti-CCR8 antibody; (ii) a chemotherapeutic agent, and (iii) a PD-1 inhibitor or PD-L1 inhibitor (e.g. an anti-PD-1 antibody or anti-PD-L1 antibody); wherein the anti-CCR8 antibody has antibody-dependent cellular cytotoxicity (ADCC) activity and/or complement-dependent cytotoxicity (CDC) activity, and wherein the anti-CCR8 antibody is optionally a CCR8 neutralizing antibody.

In some embodiments, the chemotherapeutic agent is co-administered at a lower dose than in a standard of care chemotherapeutic regimen that does not comprise an anti-CCR8 antibody.

In another aspect, provided herein is a method of treating cancer in a subject comprising co-administering to the subject an effective amount of (i) an anti-CCR8 antibody; (ii) a chemotherapeutic agent, and (iii) optionally a PD-1 inhibitor or PD-L1 inhibitor (e.g., an anti-PD-1 antibody or anti-PD-L1 antibody); wherein the anti-CCR8 antibody has antibody-dependent cellular cytotoxicity (ADCC) activity and/or complement-dependent cytotoxicity (CDC) activity; wherein the anti-CCR8 antibody is optionally a CCR8 neutralizing antibody, and wherein the chemotherapeutic agent is co-administered at a lower dose than in a standard of care chemotherapeutic regimen that does not comprise an anti-CCR8 antibody.

In some embodiments, the chemotherapeutic agent is one chemotherapeutic agent. In some embodiments, the chemotherapeutic agent is a plurality of chemotherapeutic agents. In some embodiments, the chemotherapeutic agent is selected from the group consisting of a platinum complex, a taxane, pemetrexed, gemcitabine, fluorouracil, irinotecan, etoposide, and doxorubicin. In some embodiments, the chemotherapeutic agent comprises a platinum complex. In some embodiments, the platinum complex is selected from the group consisting of carboplatin, cisplatin, and oxaliplatin. In some embodiments, the chemotherapeutic agent comprises gemcitabine. In some embodiments, the chemotherapeutic agent comprises a taxane. In some embodiments the taxane is docetaxel. In some embodiments, the chemotherapeutic agent is administered at a dose that is 90% or lower, 80% or lower, 70% or lower, 60% or lower, 50% or lower, 50% or lower, 40% or lower, 30% or lower, or 20% or lower than the dose of the chemotherapeutic agent administered in a standard of care regimen that does not comprise an anti-CCR8 antibody.

In some embodiments the cancer comprises a solid tumor.

In some embodiments, the cancer comprises tumor-infiltrating Treg cells that express CCR8. In some embodiments, the CCR8 is expressed on the surface of the Treg cells at fewer than 10,000 copies per cell (as determined fluorescence-activated cell sorting (FACS) and/or flow cytometry).

In some embodiments the cancer is selected from the group consisting of breast cancer, colorectal cancer, head and neck cancer, lung cancer, ovarian cancer, gastric cancer, stomach adenocarcinoma, and thymoma. In some embodiments the cancer is selected from the group consisting of endometrial adenocarcinoma, colorectal cancer, ovarian cancer, vaginal squamous cell carcinoma, endometrial adenocarcinoma, colorectal cancer, melanoma (e.g. cutaneous), pancreatic cancer, small-cell lung cancer (SCLC), non-small cell lung cancer (NSCLC), uterine leiomyosacoma, cholangiocarcinoma, adenoid cyctic carcinoma, cervical cancer, renal cell carcinoma (RCC), anal cancer, esophagogastric junction (EGJ) adenocarcinoma, and gastric adenocarcinoma. In some embodiments, the cancer is selected from the group consisting of head and neck squamous cell carcinoma (HNSCC), non-small cell lung cancer (NSCLC), gastric adenocarcinoma, EGJ adenocarcinoma, and colorectal cancer (CRC) (e.g., microsatelite-stable (MSS) mCRC). In some embodiments, the cancer is selected from the group consisting of breast cancer, pancreatic cancer, and lung cancer. In some embodiments, the breast cancer is selected from triple-negative breast cancer (TNBC), HR⁺/HER²⁻ breast cancer, or HR⁺/HER2^{low} breast cancer. In some embodiments, the pancreatic cancer is pancreatic ductal adenocarcinoma (PDAC). In some embodiments the cancer is lung cancer. In some embodiments, the lung cancer is non-small cell lung cancer (NSCLC) or small cell lung cancer (SCLC). In some embodiments the lung cancer is NSCLC. In some embodiments, the cancer is metastatic.

In some embodiments, the the cancer is ovarian cancer and the co-administered chemotherapeutic agent is selected from the group consisting of 5-flourouracil, albumin bound paclitaxel, altretamine, anastrozole, capecitabine, carboplatin, cisplatin, cyclophosphamide, docetaxel, doxorubicin, etoposide, exemestane, gemcitabine, ifosfamide, irinotecan, letrozole, leuprolide acetate, liposomal doxorubicin, megestrol acetate, melphalan, olaparib, oxaliplatin, paclitaxel, pazopanib, pemetrexed, tamoxifen, topotecan, vinorelbine, and any combinations thereof.

In some embodiments, the cancer is HNSCC and the co-administered chemotherapeutic agent is selected from the group consisting of afatinib, bleomycin, capecitabine, carboplatin, cetuximab, cisplatin, docetaxel, fluorouracil, gemcitabine, hydroxyurea, methotrexate, nivolumab, paclitaxel, vinorelbine, and any combinations thereof.

In some embodiments, the cancer is gastric adenocarcinoma and the co-administered chemotherapeutic agent is selected from the group consisting of capecitabine, carboplatin, cisplatin, docetaxel, epirubicin, fluoropyrimidine, fluorouracil, irinotecan, leucovorin, mitomycin, oxaliplatin, paclitaxel, and any combinations thereof.

In some embodiments, the cancer is esophagogastric junction (EGJ) adenocarcinoma and the co-administered chemotherapeutic agent is selected from the group consisting of capecitabine, carboplatin, cisplatin, docetaxel, epirubicin, fluoropyrimidine, fluorouracil, irinotecan, leucovorin, oxaliplatin, paclitaxel, and any combinations thereof.

In some embodiments, the cancer is colorectal cancer and the co-administered chemotherapeutic agent is selected from the group consisting of capecitabine, cetuximab, fluorouracil, irinotecan, leucovorin, oxaliplatin, panitumumab, ziv-aflibercept, and any combinations thereof.

In some embodiments, the cancer is breast cancer and the co-administered chemotherapeutic agent is selected from the group consisting of albumin-bound paclitaxel, anastrozole, atezolizumab, capecitabine, carboplatin, cisplatin, cyclophosphamide, docetaxel, doxorubicin, epirubicin, everolimus, exemestane, fluorouracil, fulvestrant, gemcitabine, ixabepilone, lapatinib, letrozole, methotrexate, mitoxantrone, paclitaxel, pegylated liposomal doxorubicin, pertuzumab, tamoxifen, toremifene, trastuzumab, vinorelbine, and any combinations thereof.

In some embodiments, the breast cancer is is TNBC and the co-administered chemotherapeutic agent is selected from the group consisting of cyclophosphamide, docetaxel, doxorubicin, epirubicin, fluorouracil, paclitaxel, and any combinations thereof.

In some embodiments the lung cancer is NSCLC and the co-administered chemotherapeutic agent is selected from the group consisting of afatinib, albumin-bound paclitaxel, alectinib, cabozantinib, carboplatin, cisplatin, crizotinib, dabrafenib, docetaxel, erlotinib, etoposide, gemcitabine, paclitaxel, pemetrexed, vandetanib, vemurafenib, vinblastine, vinorelbine, and any combinations thereof.

In some embodiments, the lung cancer is SCLC and the co-administered chemotherapeutic agent is selected from the group consisting of 5-flourouracil, albumin bound paclitaxel, altretamine, anastrozole, capecitabine, carboplatin, cisplatin, cyclophosphamide, docetaxel, doxorubicin, etoposide, exemestane, gemcitabine, ifosfamide, irinotecan, letrozole, leuprolide acetate, liposomal doxorubicin, megestrol acetate, melphalan, olaparib, oxaliplatin, paclitaxel, pazopanib, pemetrexed, tamoxifen, topotecan, vinorelbine, and any combinations thereof.

In some embodiments, the cancer is a CCR8 expressing blood cancer. In some embodiments, the blood cancer is selected from the group consisting of T-cell adult acute lymphocytic leukemia, T-cell childhood acute lymphocytic leukemia, lymphoblastic lymphoma, acute lymphocytic leukemia, cutaneous T cell lymphoma (CTCL), T-cell acute lymphocytic leukemia, adult T cell leukemia/lymphoma, T cell lymphoblastic leukemia/lymphoma, and anaplastic large cell lymphoma. In some embodiments, the blood cancer is CTCL.

In some embodiments, the subject is human. In some embodiments, the subject is treatment naïve. In some embodiments, the subject has received one or more courses of anticancer treatments, and optionally wherein the cancer has progressed on one or more courses of anticancer treatments. In some embodiments, the anticancer treatment is selected from the group consisting of surgery, radiation therapy, a hormone therapy, a targeted anticancer agent, a chemotherapeutic agent, an immunotherapy, and an antibody drug conjugate (ADC). In some embodiments, the chemotherapeutic agent is selected from the group consisting of a platinum complex, a taxane, pemetrexed, gemcitabine, fluorouracil, irinotecan, etoposide, and doxorubicin. In some embodiments, the platinum complex is selected from the group consisting of carboplatin, cisplatin, and oxaliplatin. In some embodiments, the taxane is docetaxel. In some embodiments, the immunotherapy comprises an anti-PD-1 antibody or an anti-PD-L1 antibody. In some embodiments, the anti-PD-1 antibody or anti-PD-L1 antibody is selected from the group consisting of pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, and zimberelimab. In some embodiments, the immunotherapy further comprises an anti-TIGIT antibody. In some embodiments, the anti-TIGIT antibody is selected from the group consisting of tiragolumab, vibostolimab, domvanalimab, AB308, AK127, BMS-986207, or etigilimab.

In some embodiments, the anti-CCR8 antibody comprises: (a) an HCDR1 comprising the amino acid sequence of SEQ ID NO: 12, an HCDR2 comprising the amino acid sequence of SEQ ID NO: 13, an HCDR3 comprising the amino acid sequence of SEQ ID NO: 14, an LCDR1 comprising the amino acid sequence of SEQ ID NO: 15, an LCDR2 comprising the amino acid sequence of SEQ ID NO: 16, and an LCDR3 comprising the amino acid sequence of SEQ ID NO: 17; (b) an HCDR1 comprising the amino acid sequence of SEQ ID NO: 24, an HCDR2 comprising the amino acid sequence of SEQ ID NO: 25, an HCDR3 comprising the amino acid sequence of SEQ ID NO: 26, an LCDR1 comprising the amino acid sequence of SEQ ID NO: 27, an LCDR2 comprising the amino acid sequence of SEQ ID NO: 28, and an LCDR3 comprising the amino acid sequence of SEQ ID NO: 29; (c) an HCDR1 comprising the amino acid sequence of SEQ ID NO: 36, an HCDR2 comprising the amino acid sequence of SEQ ID NO: 37, an HCDR3 comprising the amino acid sequence of SEQ ID NO: 38, an LCDR1 comprising the amino acid sequence of SEQ ID NO: 39, an LCDR2 comprising the amino acid sequence of SEQ ID NO: 40, and an LCDR3 comprising the amino acid sequence of SEQ ID NO: 41; (d) an HCDR1 comprising the amino acid sequence of SEQ ID NO: 48, an HCDR2 comprising the amino acid sequence of SEQ ID NO: 49, an HCDR3 comprising the amino acid sequence of SEQ ID NO: 50, an LCDR1 comprising the amino acid sequence of SEQ ID NO: 51, an LCDR2 comprising the amino acid sequence of SEQ ID NO: 52, and an LCDR3 comprising the amino acid sequence of SEQ ID NO: 53; or (e) an HCDR1 comprising the amino acid sequence of SEQ ID NO: 60, an HCDR2 comprising the amino acid sequence of SEQ ID NO: 61, 72, or 78, an HCDR3 comprising the amino acid sequence of SEQ ID NO: 62, 73, or 79, an LCDR1 comprising the amino acid sequence of SEQ ID NO: 63, an LCDR2 comprising the amino acid sequence of SEQ ID NO: 64, and an LCDR3 comprising the amino acid sequence of SEQ ID NO: 65; or (f) an HCDR1 comprising the amino acid sequence of SEQ ID NO: 84 or 100, an HCDR2 comprising the amino acid sequence of SEQ ID NO: 85, an HCDR3 comprising the amino acid sequence of SEQ ID NO: 86, an LCDR1 comprising the amino acid sequence of SEQ ID NO: 87, an LCDR2 comprising the amino acid sequence of SEQ ID NO: 88, and an LCDR3 comprising the amino acid sequence of SEQ ID NO: 89. In some embodiments, the anti-CCR8 antibody comprises: (a) a heavy chain variable region (VH) comprising an amino acid sequence that is at least 90%, 91%, 92%, 93%, 94%, 95%, 96%, 97%, 98%, or 99% identical to the amino acid sequence of SEQ ID NO: 68 or 74, and a light chain variable region (VL) comprising an amino acid sequence that is at least 90%, 91%, 92%, 93%, 94%, 95%, 96%, 97%, 98%, or 99% identical to the amino acid sequence of SEQ ID NO: 69 or 75; or (b) a heavy chain variable region (VH) comprising an amino acid sequence that is at least 90%, 91%, 92%, 93%, 94%, 95%, 96%, 97%, 98%, or 99% identical to the amino acid sequence of SEQ ID NO: 92 or 96, and a light chain variable region (VL) comprising an amino acid sequence that is at least 90%, 91%, 92%, 93%, 94%, 95%, 96%, 97%, 98%, or 99% identical to the amino acid sequence of SEQ ID NO: 93 or 97. In some embodiments, the anti-CCR8 antibody comprises: (a) a heavy chain variable region (VH) comprising the amino acid sequence of SEQ ID NO: 68 or 74, and a light chain variable region (VL) comprising the amino acid sequence of SEQ ID NO: 69 or 75; or (b) a heavy chain variable region (VH) comprising the amino acid sequence of SEQ ID NO: 92 or 96, and a light chain variable region (VL) comprising the amino acid sequence of SEQ ID NO: 93 or 97. In some embodiments, the anti-CCR8 antibody is a monoclonal antibody. In some embodiments, the anti-CCR8 antibody is a humanized antibody. In some embodiments, the anti-CCR8 antibody is a full-length antibody. In some embodiments, the anti-CCR8 antibody is an IgG1 or IgG3 antibody. In some embodiments, the anti-CCR8 antibody comprises: (a) a heavy chain (HC) comprising an amino acid sequence that is at least 90%, 91%, 92%, 93%, 94%, 95%, 96%, 97%, 98%, or 99% identical to the amino acid sequence of SEQ ID NO: 70 or 76, and a light chain (LC) comprising an amino acid sequence that is at least 90%, 91%, 92%, 93%, 94%, 95%, 96%, 97%, 98%, or 99% identical to the amino acid sequence of SEQ ID NO: 71 or 77; or (b) a heavy chain (HC) comprising an amino acid sequence that is at least 90%, 91%, 92%, 93%, 94%, 95%, 96%, 97%, 98%, or 99% identical to the amino acid sequence of SEQ ID NO: 94 or 98, and a light chain (LC) comprising an amino acid sequence that is at least 90%, 91%, 92%, 93%, 94%, 95%, 96%, 97%, 98%, or 99% identical to the amino acid sequence of SEQ ID NO: 95 or 99. In some embodiments, the anti-CCR8 antibody comprises: (a) a heavy chain (HC) comprising the amino acid sequence of SEQ ID NO: 70 or 76, and a light chain (LC) comprising the amino acid sequence of SEQ ID NO: 71 or 77; or (b) a heavy chain (HC) comprising the amino acid sequence of SEQ ID NO: 94 or 98, and a light chain (LC) comprising the amino acid sequence of SEQ ID NO: 95 or 99. In some embodiments, the anti-CCR8 antibody is an afucosylated antibody. In some embodiments, the anti-CCR8 antibody comprises a heavy chain constant region mutation at one or more positions selected from L234, L235, G236, S239, F243, H268, D270, R292, S298, Y300, V305, K326, A330, 1332, E333, K334, and P396. In some embodiments, the anti-CCR8 antibody comprises a heavy chain constant region mutation selected from S239D, S239M, F243L, H268D, D270E, R292P, S298A, Y300L, V305I, K326D, A330L, A330M, 1332E, E333A, K334A, K334E, and P396L. In some embodiments, the anti-CCR8 antibody comprises a heavy chain constant region mutations selected from F243L/R292P/Y300L/V305I/P396L, S239D/1332E, S239D/1332E/A330L, S298A/E333A/K334A, L234Y/L235Q/G236W/S239M/H268D/D270E/S298A, and D270E/K326D/A330M/K334E. In some embodiments, the anti-CCR8 antibody inhibits binding of CCL1 to CCR8. In some embodiments, the anti-CCR8 antibody is selected from the group consisting of BMS-986340 (Bristol Myers Squibb), LM-108 (LaNova Medicines), S-531011 (Shionogi), FPA157 (Five Prime, Amgen), IPG-7236 (Immunophage Biomedical), ICP-B05 (InnoCare Pharma Tech), SRF-114 (Surface Oncology), HBM1022 (Harbour BioMed), HFB1011 (HiFiBio), BAY-3375968 (Bayer), IO-1 (Oncurious), ZL-1218 (Zai Lab), GB2101 (Genor), and PSB-114 (Sound Biologics).

In some embodiments the co-administered PD-1 inhibitor or PD-L1 inhibitor is an anti-PD1 antibody or anti-PD-L1 antibody. In some embodiments, the co-administered anti-PD-1 antibody or anti-PD-L1 antibody is selected from the group consisting of pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, and zimberelimab. In some embodiments the co-administered PD-1 inhibitor or PD-L1 inhibitor is a small molecule. In some embodiments the small molecule PD-1 inhibitor or PD-L1 inhibitor is selected from the group consisting of CA-170, GS-4224, GS-4416, INCB99280, INCB99318, and lazertinib.

In some embodiments, the methods provided herein further comprise co-administering to the subject one or more additional therapeutic agents.

In another aspect, provided herein is an anti-CCR8 antibody for use in combination with a chemotherapeutic agent and an anti-PD-1 antibody or anti-PD-L1 antibody in a method of treating cancer, wherein the method comprises co-administering the anti-CCR8 antibody, chemotherapeutic agent, and anti-PD1 antibody or anti-PD-L1 antibody to a subject, wherein the anti-CCR8 antibody has antibody-dependent cellular cytotoxicity (ADCC) activity and/or complement-dependent cytotoxicity (CDC) activity, and wherein the anti-CCR8 antibody is optionally a CCR8 neutralizing antibody.

In another aspect, provided herein is an anti-CCR8 antibody for use in combination with a chemotherapeutic agent, and optionally a PD-1 inhibitor or PD-L1 inhibitor (e.g., an anti-PD-1 antibody or anti-PD-L1 antibody) in a method of treating cancer, wherein the method comprises co-administering the anti-CCR8 antibody, chemotherapeutic agent, and PD-1 inhibitor or PD-L1 inhibitor (e.g., anti-PD1 antibody or anti-PD-L1 antibody) to a subject, wherein the anti-CCR8 antibody has antibody-dependent cellular cytotoxicity (ADCC) activity and/or complement-dependent cytotoxicity (CDC) activity, and wherein the anti-CCR8 antibody is optionally a CCR8 neutralizing antibody, and wherein the chemotherapeutic agent is administered at a lower dose than in a standard of care chemotherapeutic regimen that does not comprise an anti-CCR8 antibody class agent.

### BRIEF DESCRIPTION OF THE DRAWINGS

FIG. 1 shows tumor growth curves in a syngeneic mouse breast cancer model (4T1). Mice were each administered with a single dose of a control antibody, an anti-CCR8 antibody, low-dose chemotherapy (cisplatin), or a combination thereof.

FIG. 2 shows tumor growth curves in a syngeneic mouse pancreatic cancer model (Panc02). Mice were each administered with a single dose of a control antibody, an anti-CCR8 antibody, low-dose chemotherapy (gemcitabine), or a combination thereof.

FIG. 3 shows tumor growth curves in a syngeneic mouse melanoma model (B16F10). Mice were each administered with a single dose of control antibody, an anti-CCR8 antibody, low-dose chemotherapy (gemcitabine), or a combination thereof.

FIG. 4A and FIG. 4B show bar charts illustrating tumor infiltrating lymphocytes from a syngeneic mouse melanoma model (B16F10). Mice were each administered with a single dose of a control antibody, gemcitabine (“low dose SOC chemo”) and/or an anti-CCR8 antibody, and frequencies of regulatory T-cells (Tregs; FIG. 3A) or effector T-cells (FIG. 3B) were quantified. T test results are shown, wherein **** is p<0.0001, *** p<0.001, ** is p<0.01 and * is p<0.1.

FIG. 5 shows a tumor growth curve in a syngeneic mouse melanoma model (B16F10). Mice were each administered with a single dose of a control antibody, an anti-CCR8 antibody, an anti-PD-1 antibody, or a combination thereof.

FIG. 6 shows a tumor growth curve in a syngeneic mouse lung adenocarcinoma model (LLC). Mice were each administered with a single dose of a control antibody, an anti-CCR8 antibody, low dose chemotherapy (docetaxel), or a combination thereof.

FIG. 7 shows tumor growth curve in a syngeneic mouse lung adenocarcinoma model (LLC). Mice were each administered with a single dose of a control antibody, an anti-CCR8 antibody, low dose chemotherapy (docetaxel), an anti-PD-1 antibody, an anti-CCR8 antibody/chemotherapy combination, an anti-PD-1 antibody/chemotherapy combination, or an anti-CCR8 antibody/anti-PD-1 antibody/chemotherapy combination.

FIG. 8 shows a bar graph illustrating the tumor volumes on day 15 for the LLC model treatment cohorts shown in FIG. 7.

### DETAILED DESCRIPTION

### Definitions

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

As is understood by one skilled in the art, reference to “about” a value or parameter herein includes (and describes) embodiments that are directed to that value or parameter per se. For example, description referring to “about X” includes description of “X”.

“CCR8” and “C—C chemokine receptor type 8” and “chemokine receptor 8,” as used herein refer to any native CCR8 that results from expression and processing of CCR8 in a cell. The term includes CCR8 from any vertebrate source, including mammals such as primates (e.g., humans and cynomolgus monkeys) and rodents (e.g., mice and rats), unless otherwise indicated. The term also includes naturally occurring variants of CCR8, e.g., splice variants or allelic variants. The amino acid sequence of an exemplary human CCR8 protein is shown in SEQ ID NO: 101 (UniProt Identifier P51685). The amino acid sequence of an exemplary mouse CCR8 protein is shown in SEQ ID NO: 102 (UniProt Identifier P56484). The amino acid sequence of an exemplary cynomolgus monkey CCR8 protein is shown in SEQ ID NO: 103 (UniProt Identifier G7NYJ2).

“CCL1” and “C—C motif chemokine 1,” as used herein refer to any native CCR1 that results from expression and processing of CCR1 in a cell. The term includes CCR1 from any vertebrate source, including mammals such as primates (e.g., humans and cynomolgus monkeys) and rodents (e.g., mice and rats), unless otherwise indicated. The term also includes naturally occurring variants of CCR1, e.g., splice variants or allelic variants. The amino acid sequence of an exemplary human CCR1 protein is shown in SEQ ID NO: 2 (UniProt Identifier P22362.1). An exemplary mature CCR1 protein comprises amino acids 24-96 of SEQ ID NO: 2.

As used herein, a “7-B16 antibody” should be understood as any antibody that binds to CCR8 and comprises (i) a heavy chain comprising SEQ ID NO:82 and a light chain comprising SEQ ID NO: 83, (ii) a variable heavy chain region comprising SEQ ID NO: 80 and a variable light chain region comprising SEQ ID NO: 81, or (iii) an HCDR1, an HCDR2, and an HCDR 3 comprising SEQ ID NOs: 84, 85, and 86, respectively, and an LCDR1, an LCDR2, and an LCDR 3 comprising SEQ ID NOs: 87, 88, and 89, respectively; as well as chimeric, human, or humanized versions of any of the foregoing (i), (ii), or (iii). In some embodiments, a “7-B16 antibody” may be used to specifically refer to an antibody comprising a heavy chain of SEQ ID NO: 82 and a light chain of SEQ ID NO: 83.

As used herein, the terms “anti-PD-1 antibody” or “anti-PD-L1 antibody” refer to antibodies that a) bind to programmed cell death protein 1 (PD-1, CD279; NCBI Gene ID: 5133) or programmed death-ligand 1 (PD-L1, CD274; NCBI Gene ID: 29126); and b) inhibit the PD-1/PD-L1 interaction and PD-1/PD-L1 pathway. The PD-1/PD-L1 pathway and its role in cancer immunotherapy is described, for example, in Salmaninejad et al, J. Cell Physiol (2019) 234 (10): 16824-16837. Anti-PD-1 antibodies or anti-PD-L1 antibodies that can be used in the methods provided herein include, for example, pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, and zimberelimab. In some embodiments the anti-PD-1 antibody is zimberelimab.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a CCR8 epitope is an antibody that binds this epitope with greater affinity, avidity, more readily, and/or with greater duration than it binds to other CCR8 epitopes or non-CCR8 epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

As used herein, “substantially pure” refers to material which is at least 50% pure (that is, free from contaminants), more preferably, at least 90% pure, more preferably, at least 95% pure, yet more preferably, at least 98% pure, and most preferably, at least 99% pure.

As used herein, the term “epitope” refers to a site on a target molecule (for example, an antigen, such as a protein, nucleic acid, carbohydrate, or lipid) to which an antigen-binding molecule (for example, an antibody, antibody fragment, or scaffold protein containing antibody binding regions) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some examples an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between an antibody residue and an antigen residue. An epitope can be identified by various scans as well, for example, an alanine or arginine scan can indicate one or more residues that the antigen-binding molecule can interact with. Unless explicitly denoted, a set of residues as an epitope does not exclude other residues from being part of the epitope for a particular antibody. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

The term “antibody” herein is used in the broadest sense and encompasses various antibody structures, including but not limited to monoclonal antibodies, polyclonal antibodies, multispecific antibodies (for example, bispecific (such as Bi-specific T-cell engagers) and trispecific antibodies), and antibody fragments so long as they exhibit the desired antigen-binding activity.

The term antibody includes, but is not limited to, fragments that are capable of binding to an antigen, such as Fv, single-chain Fv (scFv), Fab, Fab′, di-scFv, sdAb (single domain antibody) and (Fab′)₂ (including a chemically linked F(ab′)₂). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. The term antibody also includes, but is not limited to, chimeric antibodies, humanized antibodies, and antibodies of various species such as mouse, human, cynomolgus monkey, etc. Furthermore, for all antibody constructs provided herein, variants having the sequences from other organisms are also contemplated. Thus, if a human version of an antibody is disclosed, one of skill in the art will appreciate how to transform the human sequence-based antibody into a mouse, rat, cat, dog, horse, etc. sequence. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, human scFv or a mouse scFv). This denotes the sequences of at least part of the non-CDR regions, rather than the source of the construct.

The term “monoclonal antibody” refers to an antibody of a substantially homogeneous population of antibodies, that is, the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, the contact definition, and/or a combination of the Kabat, Chothia, AbM, and/or contact definitions. Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, MD (1991)). The AbM definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, H26-H35B of H1, 50-58 of H2, and 95-102 of H3. The Contact definition can include, for example, CDRs (CDR-L1, CDR-L2, CDR-L3, CDR-H1, CDR-H2, and CDR-H3) at amino acid residues 30-36 of L1, 46-55 of L2, 89-96 of L3, 30-35 of H1, 47-58 of H2, and 93-101 of H3. The Chothia definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, 26-32 . . . 34 of H1, 52-56 of H2, and 95-102 of H3. CDRs can also be provided as shown in any one or more of the accompanying figures. With the exception of CDR1 in V_{H}, CDRs generally comprise the amino acid residues that form the hypervariable loops. The various CDRs within an antibody can be designated by their appropriate number and chain type, including, without limitation as: a) CDR-L1, CDR-L2, CDR-L3, CDR-H1, CDR-H2, and CDR-H3; b) CDRL1, CDRL2, CDRL3, CDRH1, CDRH2, and CDRH3; c) LCDR-1, LCDR-2, LCDR-3, HCDR-1, HCDR-2, and HCDR-3; or d) LCDR1, LCDR2, LCDR3, HCDR1, HCDR2, and HCDR3; etc. The term “CDR” is used herein to also encompass HVR or a “hyper variable region”, including hypervariable loops. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).)

The term “heavy chain variable region” as used herein refers to a region comprising at least three heavy chain CDRs. In some embodiments, the heavy chain variable region includes the three CDRs and at least FR2 and FR3. In some embodiments, the heavy chain variable region includes at least heavy chain HCDR1, framework (FR) 2, HCDR2, FR3, and HCDR3. In some embodiments, a heavy chain variable region also comprises at least a portion of an FR1 and/or at least a portion of an FR4.

The term “heavy chain constant region” as used herein refers to a region comprising at least three heavy chain constant domains, C_{H}1, C_{H}2, and C_{H}3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include E and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an E constant region is an IgE antibody. Certain isotypes can be further subdivided into subclasses. For example, IgG antibodies include, but are not limited to, IgG1 (comprising a γ₁ constant region), IgG2 (comprising a γ2 constant region), IgG3 (comprising a γ₃ constant region), and IgG4 (comprising a γ4 constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising at least three light chain CDRs. In some embodiments, the light chain variable region includes the three CDRs and at least FR2 and FR3. In some embodiments, the light chain variable region includes at least light chain LCDR1, framework (FR) 2, LCDR2, FR3, and LCDR3. For example, a light chain variable region may comprise light chain CDR1, framework (FR) 2, CDR2, FR3, and CDR3. In some embodiments, a light chain variable region also comprises at least a portion of an FR1 and/or at least a portion of an FR4.

The term “light chain constant region” as used herein refers to a region comprising a light chain constant domain, C_{L}. Nonlimiting exemplary light chain constant regions include λ and κ. Of course, non-function-altering deletions and alterations within the domains are encompassed within the scope of the term “light chain constant region,” unless designated otherwise.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

An “acceptor human framework” for the purposes herein is a framework comprising the amino acid sequence of a light chain variable domain (V_{L}) framework or a heavy chain variable domain (V_{H}) framework derived from a human immunoglobulin framework or a human consensus framework, as defined below. An acceptor human framework derived from a human immunoglobulin framework or a human consensus framework can comprise the same amino acid sequence thereof, or it can contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the V_{L} acceptor human framework is identical in sequence to the V_{L} human immunoglobulin framework sequence or human consensus framework sequence.

“Affinity” refers to the strength of the sum total of noncovalent interactions between a single binding site of a molecule (for example, an antibody) and its binding partner (for example, an antigen). The affinity of a molecule X for its partner Y can generally be represented by the dissociation constant (K_{D}). Affinity can be measured by common methods known in the art (such as, for example, ELISA K_{D}, KinExA, bio-layer interferometry (BLI), and/or surface plasmon resonance devices (such as a BIAcore® device), including those described herein).

The term “K_{D}”, as used herein, refers to the equilibrium dissociation constant of an antibody-antigen interaction.

In some embodiments, the “K_{D},” “K_{d},” “Kd” or “Kd value” of the antibody is measured by using surface plasmon resonance assays using a BIACORE©-2000 or a BIACORE©-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μL/minute to achieve approximately 10 response units (RU) of coupled protein. Following the injection of antigen, 1 M ethanolamine is injected to block unreacted groups. For kinetics measurements, serial dilutions of polypeptide, for example, full length antibody, are injected in PBS with 0.05% TWEEN-20™ surfactant (PBST) at 25° C. at a flow rate of approximately 25 μL/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, for example, Chen et al., J. Mol. Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

The term “biological activity” refers to any one or more biological properties of a molecule (whether present naturally as found in vivo, or provided or enabled by recombinant means). Biological properties include, but are not limited to, binding a cytokine, inducing cell proliferation, inhibiting cell growth, inducing other cytokines, inducing apoptosis, and enzymatic activity. In some embodiments, biological activities of CCR8 include antiapoptotic activities, cell chemotaxis, immune suppressive functions, and capacity to polarize cells toward various cell differentiation paths.

A “chimeric antibody” as used herein refers to an antibody in which a portion of the heavy and/or light chain is derived from a particular source or species, while at least a part of the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species. The chimeric construct can also be a functional fragment, as noted above.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an antibody fragment, such as Fab, an scFv, a (Fab′)₂, etc. The term humanized also denotes forms of non-human (for example, murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence of non-human immunoglobulin. Humanized antibodies can include human immunoglobulins (recipient antibody) in which residues from a complementary determining region (CDR) of the recipient are substituted by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody can comprise residues that are found neither in the recipient antibody nor in the imported CDR or framework sequences, but are included to further refine and optimize antibody performance. In general, the humanized antibody can comprise substantially all of at least one, and typically two, variable domains, in which all or substantially all of the CDR regions correspond to those of a non-human immunoglobulin and all or substantially all of the FR regions are those of a human immunoglobulin consensus sequence. In some embodiments, the humanized antibody can also comprise at least a portion of an immunoglobulin constant region or domain (Fc), typically that of a human immunoglobulin. Other forms of humanized antibodies have one or more CDRs (CDR L1, CDR L2, CDR L3, CDR H1, CDR H2, and/or CDR H3) which are altered with respect to the original antibody, which are also termed one or more CDRs “derived from” one or more CDRs from the original antibody. As will be appreciated, a humanized sequence can be identified by its primary sequence and does not necessarily denote the process by which the antibody was created.

An “CDR-grafted antibody” as used herein refers to a humanized antibody in which one or more complementarity determining regions (CDRs) of a first (non-human) species have been grafted onto the framework regions (FRs) of a second (human) species.

A “human antibody” as used herein encompasses antibodies produced in humans, antibodies produced in non-human animals that comprise human immunoglobulin genes, such as XenoMouse® mice, and antibodies selected using in vitro methods, such as phage display (Vaughan et al., 1996, Nature Biotechnology, 14:309-314; Sheets et al., 1998, Proc. Natl. Acad. Sci. (USA) 95:6157-6162; Hoogenboom and Winter, 1991, J. Mol. Biol., 227:381; Marks et al., 1991, J. Mol. Biol., 222:581), wherein the antibody repertoire is based on a human immunoglobulin sequence. The term “human antibody” denotes the genus of sequences that are human sequences. Thus, the term is not designating the process by which the antibody was created, but the genus of sequences that are relevant.

A “functional Fc region” possesses an “effector function” of a native sequence Fc region. Exemplary “effector functions” include Fc receptor binding; C1q binding; CDC; ADCC; phagocytosis; down regulation of cell surface receptors (for example B cell receptor; BCR), etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see, for example, Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

“Effector functions” refer to biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (for example B cell receptor); and B cell activation.

“Human effector cells” are leukocytes which express one or more FcRs and perform effector functions. In some embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, for example, from blood.

“Antibody-dependent cell-mediated cytotoxicity” or “ADCC” refers to a form of cytotoxicity in which secreted Ig bound onto Fc receptors (FcRs) present on certain cytotoxic cells (for example NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or U.S. Pat. No. 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, for example, in an animal model such as that disclosed in Clynes et al. Proc. Natl. Acad. Sci. (USA) 95:652-656 (1998). Additional polypeptide variants with altered Fc region amino acid sequences (polypeptides with a variant Fc region) and increased or decreased ADCC activity are described, for example, in U.S. Pat. Nos. 7,923,538, and 7,994,290.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, for example, as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, for example, in U.S. Pat. No. 6,194,551 B1, U.S. Pat. Nos. 7,923,538, 7,994,290 and WO 1999/51642. See also, for example, Idusogie et al., J. Immunol. 164: 4178-4184 (2000).

A polypeptide variant with “altered” FcR binding affinity or ADCC activity is one which has either enhanced or diminished FcR binding activity and/or ADCC activity compared to a parent polypeptide or to a polypeptide comprising a native sequence Fc region. The polypeptide variant which “displays increased binding” to an FcR binds at least one FcR with better affinity than the parent polypeptide. The polypeptide variant which “displays decreased binding” to an FcR, binds at least one FcR with lower affinity than a parent polypeptide. Such variants which display decreased binding to an FcR may possess little or no appreciable binding to an FcR, for example, 0-20% binding to the FcR compared to a native sequence IgG Fc region.

The polypeptide variant which “mediates antibody-dependent cell-mediated cytotoxicity (ADCC) in the presence of human effector cells more effectively” than a parent antibody is one which in vitro or in vivo is more effective at mediating ADCC, when the amounts of polypeptide variant and parent antibody used in the assay are essentially the same. Generally, such variants will be identified using the in vitro ADCC assay as herein disclosed, but other assays or methods for determining ADCC activity, for example in an animal model etc., are contemplated.

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

The phrase “substantially different,” as used herein, denotes a sufficiently high degree of difference between two numeric values such that one of skill in the art would consider the difference between the two values to be of statistical significance within the context of the biological characteristic measured by said values. In some embodiments, the two substantially different numeric values differ by greater than about any one of 10%, 20%, 25%, 30%, 35%, 40%, 45%, 50%, 60%, 70%, 80%, 90%, or 100%.

The phrase “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 20%, 25%, 30%, 35%, 40%, 45%, 50%, 60%, 70%, 80%, 90%, or 100% compared to the reference value.

The term “leader sequence” refers to a sequence of amino acid residues located at the N-terminus of a polypeptide that facilitates secretion of a polypeptide from a mammalian cell. A leader sequence can be cleaved upon export of the polypeptide from the mammalian cell, forming a mature protein. Leader sequences can be natural or synthetic, and they can be heterologous or homologous to the protein to which they are attached.

A “native sequence” polypeptide comprises a polypeptide having the same amino acid sequence as a polypeptide found in nature. Thus, a native sequence polypeptide can have the amino acid sequence of naturally occurring polypeptide from any mammal. Such native sequence polypeptide can be isolated from nature or can be produced by recombinant or synthetic means. The term “native sequence” polypeptide specifically encompasses naturally occurring truncated or secreted forms of the polypeptide (for example, an extracellular domain sequence), naturally occurring variant forms (for example, alternatively spliced forms) and naturally occurring allelic variants of the polypeptide.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary conservative substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, 0-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E and DG44 cells, respectively. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) a provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” or “subject” are used interchangeably herein to refer to an animal, for example, a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

A “disease” or “disorder” as used herein refers to a condition where treatment is needed and/or desired.

“Cancer” and “tumor,” as used herein, are interchangeable terms that refer to any abnormal cell or tissue growth or proliferation in an animal. As used herein, the terms “cancer” and “tumor” encompass solid and hematological/lymphatic cancers as well as malignant, pre-malignant, and benign growth, such as dysplasia. Examples of cancer include but are not limited to, carcinoma, lymphoma, blastoma, sarcoma, and leukemia. More particular non-limiting examples of such cancers include squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, mesothelioma, and various types of head and neck cancer. In some embodiments, hematological/lymphatic cancers are referred to as “blood cancers.” Nonlimiting exemplary blood cancers include B- and T-cell mixed leukemia, B-cell lymphoma, chronic myeloid leukemia (CML), chronic myelomonocytic leukemia, diffuse large B-cell lymphoma (DLBC), lymphoma, mantle cell lymphoma (MCL), multiple myeloma, myelodysplastic syndromes (MDS), myeloproliferative disorders, peripheral T-cell lymphoma, T-cell leukemia, acute myeloid leukemia (AML), chronic lymphocytic leukemia (CLL), small lymphocytic lymphoma (SLL), CLL/SLL, mature T-cell and NK-cell lymphoma, follicular lymphoma, acute lymphocytic leukemia (ALL), T-cell acute lymphocytic leukemia (TALL), T-cell adult acute lymphocytic leukemia, T-cell childhood acute lymphocytic leukemia, lymphoblastic lymphoma, cutaneous T cell lymphoma (CTCL), adult T cell leukemia/lymphoma (ATLL), T cell lymphoblastic leukemia/lymphoma (TLLL), angioimmunoblastic T cell lymphoma (ATCL), hepatosplenic T cell lymphoma (HTCL), peripheral T cell lymphoma not otherwise specified (PTCL NOS), Burkitt lymphoma (BL), chronic myelomonocytic leukemia (CMML), extranodal NK/T cell lymphoma (NKTCL), primary effusion lymphoma (PEL), acute lymphocytic leukemia/acute myeloid leukemia (ALL, AML), histiocytic lymphoma (HL), marginal zone lymphoma (MZL), B-cell acute lymphocytic leukemia, and anaplastic large cell lymphoma (ALCL).

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering an anti-CCR8 antibody. “Ameliorating” also includes shortening or reduction in duration of a symptom.

In the context of cancer, the term “treating” includes any or all of: inhibiting growth of cancer cells, inhibiting replication of cancer cells, lessening of overall tumor burden and ameliorating one or more symptoms associated with the disease.

As used herein, the term “Regulatory T cells” (also known as “Tregs” or “Treg cells” or “suppressor T cells”) are a subpopulation of T cells that are immunosuppressive and generally suppress or downregulate induction and proliferation of effector T cells. Tregs express CD4, FOXP3, and CD25 (IL-2 receptor α-chain). Human Foxp3+CD4+ T cells have been divided into three subfractions based upon the expression level of Foxp3 and the cell surface molecules CD25 and CD45RA. The Foxp3hiCD45RA-CD25hi and Foxp3loCD45RA+CD251o phenotypes correspond to suppressive Treg cells, whereas the Foxp3loCD45RA-CD251o fraction marks activated T effector (Teff) cells without suppressive activity. In addition, Treg cells from cancer patients, as compared to those in healthy subjects, are usually characterized by a distinct expression profile of chemokine receptors, such as CCR4, CXCR4, and CCR5, which facilitates their migration into tumors in response to the corresponding chemokine ligands derived from tumor microenvironment. See, e.g., Liu, et al., FEBS J. (2016) 283(14):2731-48 and Miyara, et al., Immunity (2009) 30, 899-911.

“Conventional T cells” or “Tconv” are a population of T cells that are generally CD4 positive (i.e., CD4+), but which are distinguishable from Tregs in that Tconv are generally FoxP3 negative (i.e., FoxP3−).

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

The term “control” refers to a composition known to not contain an analyte (“negative control”) or to contain analyte (“positive control”). A positive control can comprise a known concentration of analyte. “Control,” “positive control,” and “calibrator” may be used interchangeably herein to refer to a composition comprising a known concentration of analyte. A “positive control” can be used to establish assay performance characteristics and is a useful indicator of the integrity of reagents (for example, analytes).

“Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (for example, severity of disease, progression/non progression/improvement, etc.). While the present disclosure may provide exemplary predetermined levels, it is well-known that cutoff values may vary depending on the nature of the immunoassay (for example, antibodies employed, etc.). It further is well within the skill of one of ordinary skill in the art to adapt the disclosure herein for other immunoassays to obtain immunoassay-specific cutoff values for those other immunoassays based on this disclosure. Whereas the precise value of the predetermined cutoff/level may vary between assays, correlations as described herein (if any) may be generally applicable.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time. A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased on non-treated sample of a subject individual. In some examples, a reference is obtained from one or more healthy individuals who are not the subject or patient.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce”, “inhibit”, or “prevent” do not denote or require complete prevention over all time.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

A “therapeutically effective amount” of a substance/molecule, agonist or antagonist may vary according to factors such as the disease state, age, sex, and weight of the individual, and the ability of the substance/molecule, agonist or antagonist, to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

A “chimeric antigen receptor T cell therapy” or “CAR-T therapy” refers to a therapeutic agent comprising a T cell genetically modified to express a receptor that recognizes an antigen expressed by tumor cell. The antigen may be an antigen specifically expressed by the tumor or an antigen expressed by both cancerous cells and healthy tissue. In some embodiments CAR-T therapy is adoptive CAR-T therapy, in which a patients T cells are removed and modified to express the chimeric antigen receptor, and then returned to the patient. See, e.g., Dai et al., 2016, J Natl Cancer Inst, 108 (7): djv439, doi: 10.1093/jnci/djv439; Gill et al., 2015, Blood Rev, pii: S0268-960X(15)00080-6, doi: 10.1016/j.blre.2015.10.003; Gill et al., 2015, Immunol Rev, 263(1):68-89. doi: 10.1111/imr.12243.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes.

The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes.

As used herein, “co-administering” refers to administration of one treatment modality in addition to another treatment modality. As such, “co-administering” refers to administration of one treatment modality before, during or after administration of the other treatment modality to the subject.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

The terms “label” and “detectable label” mean a moiety attached to an antibody or its analyte to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y ⁹⁹Tc, ¹¹¹In, ¹²⁵, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

The term “conjugate” refers to an antibody that is chemically linked to a second chemical moiety, such as a therapeutic or cytotoxic agent. The term “agent” includes a chemical compound, a mixture of chemical compounds, a biological macromolecule, or an extract made from biological materials. In some embodiments, the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, the conjugate antibody may be a detectably labeled antibody used as the detection antibody.

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993); and updated versions thereof.

### Treatment Methods

This disclosure is based, at least in part, on the recognition that intra-tumoral Tregs expressing CCR8 can be bound by an anti-CCR8 antibody and selectively depleted through antibody-dependent cellular cytotoxicity (ADCC) by Natural Killer cells (NK cells). Without wishing to be bound by any theory, it is further believed that the simultaneous targeting of tumor cells with chemotherapy can trigger immunogenic cell death and lead to the uptake of tumor antigens by antigen-presenting cells (APCs). Without the immunosuppressive actions of Tregs, effector T cells (Teff) can be efficiently activated and lead to improved tumor killing. Additionally, following chronic stimulation, Teff cells can upregulate PD-1 and become dysfunctional. A combination of anti-CCR8 antibody mediated Treg depletion combined with chemotherapy and PD-1 blockade can further enhance anti-tumor T cell immunity and result in inhibition of tumor growth.

Chemotherapy has the potential to trigger immunogenic cell death and lead to enhanced T cell stimulation and activation. This disclosure is based, at least in part, on the recognition that low dose chemotherapy treatment can be combined with a Treg-depleting agent, such as an anti-CCR8 antibody, and lead to improved outcomes due to the ability of chemotherapy to induce immunogenic cell death. The combination of reduced Treg suppression and enhanced tumor antigen release during the T cell stimulation was shown to work together to lead to greater effector T cell activation and killing within the tumor microenvironment (see, e.g., Example 1).

In one aspect, provided herein is a method of treating cancer in a subject comprising co-administering to the subject an effective amount of (i) an anti-CCR8 antibody; (ii) a chemotherapeutic agent, and (iii) a PD-1 inhibitor or PD-L1 inhibitor (e.g., an anti-PD-1 antibody or anti-PD-L1 antibody); wherein the anti-CCR8 antibody has antibody-dependent cellular cytotoxicity (ADCC) activity and/or complement-dependent cytotoxicity (CDC) activity, and wherein the anti-CCR8 antibody is optionally a CCR8 neutralizing antibody. In some embodiments, the chemotherapeutic agent is co-administered at a lower dose than in a standard of care chemotherapeutic regimen that does not comprise an anti-CCR8 antibody.

In another aspect, provided herein are methods of treating cancer in a subject comprising co-administering to the subject an effective amount of (i) an anti-CCR8 antibody; (ii) a chemotherapeutic agent, and (iii) optionally a PD-1 inhibitor or PD-L1 inhibitor (e.g., an anti-PD-1 antibody or anti-PD-L1 antibody); wherein the anti-CCR8 antibody has antibody-dependent cellular cytotoxicity (ADCC) activity and/or complement-dependent cytotoxicity (CDC) activity; wherein the anti-CCR8 antibody is optionally a CCR8 neutralizing antibody, and wherein the chemotherapeutic agent is administered at a lower dose than in a standard of care chemotherapeutic regimen that does not comprise an anti-CCR8 antibody.

The administration of chemotherapeutic agents and regimens to subjects, such as human cancer patients, is well understood by physicians, e.g., clinical oncologists. Standard of care (SOC) chemotherapy regimens are provided, for example by medical associations, such as the American Society of Clinical Oncology (ASCO) and the Oncology Nursing Society (ONS) (see, e.g., Neuss et al. Chemotherapy Administration, Guidelines, Safety, Standards, Pediatric Oncology. ONF 2017, 44(1), 31-41. In some embodiments of the methods provided herein chemotherapeutic agents are administered to a subject as part of an anti-CCR8 antibody combination therapy at a lower dose than the dose used in a standard of care chemotherapeutic regimen that does not comprise an anti-CCR8 antibody. In some embodiments the chemotherapeutic agent is administered at a dose that is 90% or lower, 80% or lower, 70% or lower, 60% or lower, 50% or lower, 50% or lower, 40% or lower, 30% or lower, or 20% or lower than the dose of the chemotherapeutic agent administered in a standard of care regimen that does not comprise an anti-CCR8 antibody.

In some embodiments of the methods provided herein the co-administered chemotherapeutic agent is a single chemotherapeutic agent. In some embodiments the co-administered chemotherapeutic agent is a plurality of chemotherapeutic agents. In some embodiments the plurality of co-administered chemotherapeutic agents is two, three, four, or five chemotherapeutic agents.

In some embodiments of the methods provided herein the chemotherapeutic agent is administered at a dose that is 90% or lower, 80% or lower, 70% or lower, 60% or lower, 50% or lower, 50% or lower, 40% or lower, 30% or lower, or 20% or lower than the dose of the chemotherapeutic agent administered in a standard of care regimen that does not comprise an anti-CCR8 antibody.

In some embodiments the cancer comprises a solid tumor.

In some embodiments, the cancer comprises tumor-infiltrating Treg cells that express CCR8. In some embodiments, the CCR8 is expressed on the surface of the Treg cells at fewer than 10,000 copies per cell (as determined fluorescence-activated cell sorting (FACS) and/or flow cytometry). In some embodiments, 5%, 10%, 15%, 20%, 25%, 30%, 35%, 40%, 45%, 50%, 55%, 60%, 65%, 70%, 75%, 80%, 85%, 90%, 95%, 99%, or 100% of the tumor-infiltrating Tregs express the surface of the cell fewer than 10,000 copies of CCR8 per cell.

In some embodiments the cancer is selected from the group consisting of breast cancer, colorectal cancer, head and neck cancer, lung cancer, ovarian cancer, gastric cancer, stomach adenocarcinoma, and thymoma. In some embodiments the cancer is selected from the group consisting of endometrial adenocarcinoma, colorectal cancer, ovarian cancer, vaginal squamous cell carcinoma, endometrial adenocarcinoma, colorectal cancer, cutaneous melanoma, pancreatic cancer, small-cell lung cancer (SCLC), non-small cell lung cancer (NSCLC), uterine leiomyosacoma, cholangiocarcinoma, adenoid cyctic carcinoma, cervical cancer, anal cancer, esophagogastric junction (EGJ) adenocarcinoma, and gastric adenocarcinoma. In some embodiments, the cancer is selected from the group consisting of head and neck squamous cell carcinoma (HNSCC), non-small cell lung cancer (NSCLC), gastric adenocarcinoma, EGJ adenocarcinoma, and colorectal cancer (CRC) (e.g., microsatelite-stable (MSS) mCRC). In some embodiments, the cancer is selected from the group consisting of breast cancer, pancreatic cancer, and lung cancer. In some embodiments, the breast cancer is selected from triple-negative breast cancer (TNBC), HR⁺/HER²⁻ breast cancer, or HR⁺/HER2^{low} breast cancer. In some embodiments, the pancreatic cancer is pancreatic ductal adenocarcinoma (PDAC). In some embodiments, the lung cancer is non-small cell lung cancer (NSCLC) or small cell lung cancer (SCLC). In some embodiments, the cancer is metastatic.

In some embodiments, the cancer is a CCR8 expressing blood cancer. In some embodiments, the blood cancer is selected from the group consisting of T-cell adult acute lymphocytic leukemia, T-cell childhood acute lymphocytic leukemia, lymphoblastic lymphoma, acute lymphocytic leukemia, cutaneous T cell lymphoma (CTCL), T-cell acute lymphocytic leukemia, adult T cell leukemia/lymphoma, T cell lymphoblastic leukemia/lymphoma, and anaplastic large cell lymphoma. In some embodiments, the blood cancer is CTCL.

In some embodiments, the subject is human. In some embodiments, the subject is treatment naïve. In some embodiments, the subject has received one or more courses of anticancer treatments, and optionally wherein the cancer has progressed on one or more courses of anticancer treatments. In some embodiments, the anticancer treatment on which the cancer has progressed is selected from the group consisting of surgery, radiation therapy, a hormone therapy, a targeted anticancer agent, a chemotherapeutic agent, an immunotherapy, and an antibody drug conjugate (ADC). In some embodiments, the chemotherapeutic agent on which the cancer has progressed is selected from the group consisting of a platinum complex, a taxane, pemetrexed, gemcitabine, fluorouracil, irinotecan, etoposide, and doxorubicin. In some embodiments, the platinum complex is selected from the group consisting of carboplatin, cisplatin, and oxaliplatin. In some embodiments the taxane is paclitaxel, albumin paclitaxel, or docetaxel. In some embodiments, the taxane is docetaxel. In some embodiments, the immunotherapy on which the cancer has progressed comprises an anti-PD-1 antibody or an anti-PD-L1 antibody. In some embodiments, the anti-PD-1 antibody or anti-PD-L1 antibody is selected from the group consisting of pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, and zimberelimab. In some embodiments, the immunotherapy on which the cancer has progressed further comprises an anti-TIGIT antibody. In some embodiments, the anti-TIGIT antibody is selected from the group consisting of tiragolumab, vibostolimab, domvanalimab, AB308, AK127, BMS-986207, or etigilimab.

In another aspect, provided herein is an anti-CCR8 antibody for use in combination with a chemotherapeutic agent and a PD-1 inhibitor or PD-L1 inhibitor (e.g., an anti-PD-1 antibody or anti-PD-L1 antibody) in a method of treating cancer, wherein the method comprises co-administering the anti-CCR8 antibody, chemotherapeutic agent, and a PD-1 inhibitor or PD-L1 inhibitor (e.g., an anti-PD1 antibody or anti-PD-L1 antibody) to a subject, wherein the anti-CCR8 antibody has antibody-dependent cellular cytotoxicity (ADCC) activity and/or complement-dependent cytotoxicity (CDC) activity, and wherein the anti-CCR8 antibody is optionally a CCR8 neutralizing antibody.

In another aspect, provided herein is an anti-CCR8 antibody for use in combination with a chemotherapeutic agent, and optionally a PD-1 inhibitor or PD-L1 inhibitor (e.g., an anti-PD-1 antibody or anti-PD-L1 antibody) in a method of treating cancer, wherein the method comprises co-administering the anti-CCR8 antibody, chemotherapeutic agent, and PD-1 inhibitor or PD-L1 inhibitor (e.g., an anti-PD1 antibody or anti-PD-L1 antibody) to a subject, wherein the anti-CCR8 antibody has antibody-dependent cellular cytotoxicity (ADCC) activity and/or complement-dependent cytotoxicity (CDC) activity, and wherein the anti-CCR8 antibody is optionally a CCR8 neutralizing antibody, and wherein the chemotherapeutic agent is administered at a lower dose than in a standard of care chemotherapeutic regimen that does not comprise an anti-CCR8 antibody.

### Anti-CCR8 Antibodies

The anti-CCR8 antibodies that can be used in the methods provided herein generally have the ability to deplete CCR8 expressing target cells, such as Tregs or CCR8 expressing cancer cells. Such anti-CCR8 antibodies can include, but are not limited to, humanized antibodies, chimeric antibodies, mouse antibodies, human antibodies, and antibodies comprising the heavy chain and/or light chain CDRs discussed herein. In some embodiments, an isolated antibody that binds to CCR8 is used. In some embodiments, a monoclonal antibody that binds to CCR8 is used. In some embodiments, an anti-CCR8 antibody is an antagonist anti-CCR8 antibody. In some embodiments, an anti-CCR8 antibody used in the methods provided herein inhibits binding of CCR8 to CCL1. In some embodiments, co-administration of the anti-CCR8 antibodies described herein reduces infiltrating Treg cells in a cancer in a subject. In some embodiments, co-administration of the anti-CCR8 antibodies herein treats blood cancer that expresses CCR8.

In some embodiments, anti-CCR8 antibodies that can be used in the methods provided herein are as described in International Patent Publication No. WO 2021/163064.

In some embodiments, an anti-CCR8 antibody comprises at least one, two, three, four, five, or six CDRs selected from (a) HCDR1 comprising the amino acid sequence of SEQ ID NO: 12; (b) HCDR2 comprising the amino acid sequence of SEQ ID NO: 13; (c) HCDR3 comprising the amino acid sequence of SEQ ID NO: 14; (d) LCDR1 comprising the amino acid sequence of SEQ ID NO: 15; (e) LCDR2 comprising the amino acid sequence of SEQ ID NO: 16; and (f) LCDR3 comprising the amino acid sequence of SEQ ID NO: 17.

In some embodiments, an anti-CCR8 antibody comprises at least one, two, three, four, five, or six CDRs selected from (a) HCDR1 comprising the amino acid sequence of SEQ ID NO: 24; (b) HCDR2 comprising the amino acid sequence of SEQ ID NO: 25; (c) HCDR3 comprising the amino acid sequence of SEQ ID NO: 26; (d) LCDR1 comprising the amino acid sequence of SEQ ID NO: 27; (e) LCDR2 comprising the amino acid sequence of SEQ ID NO: 28; and (f) LCDR3 comprising the amino acid sequence of SEQ ID NO: 29.

In some embodiments, an anti-CCR8 antibody comprises at least one, two, three, four, five, or six CDRs selected from (a) HCDR1 comprising the amino acid sequence of SEQ ID NO: 36; (b) HCDR2 comprising the amino acid sequence of SEQ ID NO: 37; (c) HCDR3 comprising the amino acid sequence of SEQ ID NO: 38; (d) LCDR1 comprising the amino acid sequence of SEQ ID NO: 39; (e) LCDR2 comprising the amino acid sequence of SEQ ID NO: 40; and (f) LCDR3 comprising the amino acid sequence of SEQ ID NO: 41.

In some embodiments, an anti-CCR8 antibody comprises at least one, two, three, four, five, or six CDRs selected from (a) HCDR1 comprising the amino acid sequence of SEQ ID NO: 48; (b) HCDR2 comprising the amino acid sequence of SEQ ID NO: 49; (c) HCDR3 comprising the amino acid sequence of SEQ ID NO: 50; (d) LCDR1 comprising the amino acid sequence of SEQ ID NO: 51; (e) LCDR2 comprising the amino acid sequence of SEQ ID NO: 52; and (f) LCDR3 comprising the amino acid sequence of SEQ ID NO: 53.

In some embodiments, an anti-CCR8 antibody comprises at least one, two, three, four, five, or six CDRs selected from (a) HCDR1 comprising the amino acid sequence of SEQ ID NO: 60; (b) HCDR2 comprising the amino acid sequence of SEQ ID NO: 61, 72, or 78; (c) HCDR3 comprising the amino acid sequence of SEQ ID NO: 62, 73, or 79; (d) LCDR1 comprising the amino acid sequence of SEQ ID NO: 63; (e) LCDR2 comprising the amino acid sequence of SEQ ID NO: 64; and (f) LCDR3 comprising the amino acid sequence of SEQ ID NO: 65.

In some embodiments, an anti-CCR8 antibody comprises at least one, two, three, four, five, or six CDRs selected from (a) HCDR1 comprising the amino acid sequence of SEQ ID NO: 84 or 100; (b) HCDR2 comprising the amino acid sequence of SEQ ID NO: 85; (c) HCDR3 comprising the amino acid sequence of SEQ ID NO: 86; (d) LCDR1 comprising the amino acid sequence selected of SEQ ID NO: 87; (e) LCDR2 comprising the amino acid sequence of SEQ ID NO: 88; and (f) LCDR3 comprising the amino acid sequence of SEQ ID NO: 89.

In some embodiments, an anti-CCR8 antibody comprises a heavy chain variable region and a light chain variable region. In some embodiments, an anti-CCR8 antibody comprises at least one heavy chain comprising a heavy chain variable region and at least a portion of a heavy chain constant region, and at least one light chain comprising a light chain variable region and at least a portion of a light chain constant region. In some embodiments, an anti-CCR8 antibody comprises two heavy chains, wherein each heavy chain comprises a heavy chain variable region and at least a portion of a heavy chain constant region, and two light chains, wherein each light chain comprises a light chain variable region and at least a portion of a light chain constant region. As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some embodiments, the heavy chain is the region of the anti-CCR8 antibody that comprises the three heavy chain CDRs. In some embodiments, the light chain is the region of the anti-CCR8 antibody that comprises the three light chain CDRs.

In some embodiments, the anti-CCR8 antibody comprises six CDRs including (a) HCDR1 comprising the amino acid sequence of SEQ ID NO: 12; (b) HCDR2 comprising the amino acid sequence of SEQ ID NO: 13; (c) HCDR3 comprising the amino acid sequence of SEQ ID NO: 14; (d) LCDR1 comprising the amino acid sequence of SEQ ID NO: 15; (e) LCDR2 comprising the amino acid sequence of SEQ ID NO: 16; and (f) LCDR3 comprising the amino acid sequence of SEQ ID NO: 17.

In some embodiments, the anti-CCR8 antibody comprises six CDRs including (a) HCDR1 comprising the amino acid sequence of SEQ ID NO: 24; (b) HCDR2 comprising the amino acid sequence of SEQ ID NO: 25; (c) HCDR3 comprising the amino acid sequence of SEQ ID NO: 26; (d) LCDR1 comprising the amino acid sequence of SEQ ID NO: 27; (e) LCDR2 comprising the amino acid sequence of SEQ ID NO: 28; and (f) LCDR3 comprising the amino acid sequence of SEQ ID NO: 29.

In some embodiments, the anti-CCR8 antibody comprises six CDRs including (a) HCDR1 comprising the amino acid sequence of SEQ ID NO: 36; (b) HCDR2 comprising the amino acid sequence of SEQ ID NO: 37; (c) HCDR3 comprising the amino acid sequence of SEQ ID NO: 38; (d) LCDR1 comprising the amino acid sequence of SEQ ID NO: 39; (e) LCDR2 comprising the amino acid sequence of SEQ ID NO: 40; and (f) LCDR3 comprising the amino acid sequence of SEQ ID NO: 41.

In some embodiments, the anti-CCR8 antibody comprises six CDRs including (a) HCDR1 comprising the amino acid sequence of SEQ ID NO: 48; (b) HCDR2 comprising the amino acid sequence of SEQ ID NO: 49; (c) HCDR3 comprising the amino acid sequence of SEQ ID NO: 50; (d) LCDR1 comprising the amino acid sequence of SEQ ID NO: 51; (e) LCDR2 comprising the amino acid sequence of SEQ ID NO: 52; and (f) LCDR3 comprising the amino acid sequence of SEQ ID NO: 53.

In some embodiments, the anti-CCR8 antibody comprises six CDRs including (a) HCDR1 comprising the amino acid sequence of SEQ ID NO: 60; (b) HCDR2 comprising the amino acid sequence of SEQ ID NO: 61, 72, or 78; (c) HCDR3 comprising the amino acid sequence of SEQ ID NO: 62, 73, or 79; (d) LCDR1 comprising the amino acid sequence of SEQ ID NO: 63; (e) LCDR2 comprising the amino acid sequence of SEQ ID NO: 64; and (f) LCDR3 comprising the amino acid sequence of SEQ ID NO: 65.

In some embodiments, the anti-CCR8 antibody comprises six CDRs including (a) HCDR1 comprising the amino acid sequence of SEQ ID NO: 84 or 100; (b) HCDR2 comprising the amino acid sequence of SEQ ID NO: 85; (c) HCDR3 comprising the amino acid sequence of SEQ ID NO: 86; (d) LCDR1 comprising the amino acid sequence of SEQ ID NO: 87; (e) LCDR2 comprising the amino acid sequence of SEQ ID NO: 88; and (f) LCDR3 comprising the amino acid sequence of SEQ ID NO: 89.

In some embodiments, the anti-CCR8 antibody comprises the six CDRs as described above and binds to CCR8. In some embodiments, the anti-CCR8 antibody comprises the six CDRs as described above, binds to CCR8 and inhibits binding of CCR8 to CCL1. In some embodiments, the anti-CCR8 antibody comprises the six CDRs as described above, binds to CCR8 and enhances an immune response in a subject, and/or increases activation of T cells in a subject following administration of the antibody to the subject.

In some embodiments, an anti-CCR8 antibody is used that competes with an anti-CCR8 antibody described herein for binding to CCR8. In some embodiments, an antibody that competes for binding with any of the antibodies described herein can be made and/or used.

In some embodiments, the anti-CCR8 antibody comprises at least one, at least two, or all three VH CDR sequences selected from (a) HCDR1 comprising the amino acid sequence of SEQ ID NO: 12; (b) HCDR2 comprising the amino acid sequence of SEQ ID NO: 13; (c) HCDR3 comprising the amino acid sequence of SEQ ID NO: 14.

In some embodiments, the anti-CCR8 antibody comprises at least one, at least two, or all three VH CDR sequences selected from (a) HCDR1 comprising the amino acid sequence of SEQ ID NO: 24; (b) HCDR2 comprising the amino acid sequence of SEQ ID NO: 25; (c) HCDR3 comprising the amino acid sequence of SEQ ID NO: 26.

In some embodiments, the anti-CCR8 antibody comprises at least one, at least two, or all three VH CDR sequences selected from (a) HCDR1 comprising the amino acid sequence of SEQ ID NO: 36; (b) HCDR2 comprising the amino acid sequence of SEQ ID NO: 37; (c) HCDR3 comprising the amino acid sequence of SEQ ID NO: 38.

In some embodiments, the anti-CCR8 antibody comprises at least one, at least two, or all three VH CDR sequences selected from (a) HCDR1 comprising the amino acid sequence of SEQ ID NO: 48; (b) HCDR2 comprising the amino acid sequence of SEQ ID NO: 49; (c) HCDR3 comprising the amino acid sequence of SEQ ID NO: 50.

In some embodiments, the anti-CCR8 antibody comprises at least one, at least two, or all three VH CDR sequences selected from (a) HCDR1 comprising the amino acid sequence of SEQ ID NO: 60; (b) HCDR2 comprising the amino acid sequence of SEQ ID NO: 61, 72, or 78; (c) HCDR3 comprising the amino acid sequence of SEQ ID NO: 62, 73, or 79.

In some embodiments, the anti-CCR8 antibody comprises at least one, at least two, or all three VH CDR sequences selected from (a) HCDR1 comprising the amino acid sequence of SEQ ID NO: 84 or 100; (b) HCDR2 comprising the amino acid sequence of SEQ ID NO: 85; and (c) HCDR3 comprising the amino acid sequence of SEQ ID NO: 86.

In some embodiments, the anti-CCR8 antibody comprises at least one, at least two, or all three VL CDR sequences selected from (a) LCDR1 comprising the amino acid sequence of SEQ ID NO: 15; (b) LCDR2 comprising the amino acid sequence of SEQ ID NO: 16; and (c) LCDR3 comprising the amino acid sequence of SEQ ID NO: 17.

In some embodiments, the anti-CCR8 antibody comprises at least one, at least two, or all three VL CDR sequences selected from (a) LCDR1 comprising the amino acid sequence of SEQ ID NO: 27; (b) LCDR2 comprising the amino acid sequence of SEQ ID NO: 28; and (c) LCDR3 comprising the amino acid sequence of SEQ ID NO: 29.

In some embodiments, the anti-CCR8 antibody comprises at least one, at least two, or all three VL CDR sequences selected from (a) LCDR1 comprising the amino acid sequence of SEQ ID NO: 39; (b) LCDR2 comprising the amino acid sequence of SEQ ID NO: 40; and (c) LCDR3 comprising the amino acid sequence of SEQ ID NO: 41.

In some embodiments, the anti-CCR8 antibody comprises at least one, at least two, or all three VL CDR sequences selected from (a) LCDR1 comprising the amino acid sequence of SEQ ID NO: 51; (b) LCDR2 comprising the amino acid sequence of SEQ ID NO: 52; and (c) LCDR3 comprising the amino acid sequence of SEQ ID NO: 53.

In some embodiments, the anti-CCR8 antibody comprises at least one, at least two, or all three VL CDR sequences selected from (a) LCDR1 comprising the amino acid sequence of SEQ ID NO: 63; (b) LCDR2 comprising the amino acid sequence of SEQ ID NO: 64; and (c) LCDR3 comprising the amino acid sequence of SEQ ID NO: 65.

In some embodiments, the anti-CCR8 antibody comprises at least one, at least two, or all three VL CDR sequences selected from (a) LCDR1 comprising the amino acid sequence of SEQ ID NO: 87; (b) LCDR2 comprising the amino acid sequence of SEQ ID NO: 88; and (c) LCDR3 comprising the amino acid sequence of SEQ ID NO: 89.

In some embodiments, any of the six CDRs described herein can be combined as subparts with any of the other CDRs described herein, for a total of six CDRs in a construct. Thus, in some embodiments, two CDRs from a first antibody (for example, HCDR1 and HCDR2) can be combined with four CDRs from a second antibody (HCDR3, LCDR1, LCDR2, and LCDR3). In some embodiments, two or fewer residues in one or more of the CDRs can be replaced to obtain a variant thereof. In some embodiments, two or fewer residues can be replaced in 1, 2, 3, 4, 5, or 6 of the CDRs.

In some embodiments, the anti-CCR8 antibody comprises (I) a VH domain comprising at least one, at least two, or all three VH CDR sequences selected from (a) HCDR1 comprising the amino acid sequence of SEQ ID NO: 12; (b) HCDR2 comprising the amino acid sequence of SEQ ID NO: 13; (c) HCDR3 comprising the amino acid sequence of SEQ ID NO: 14; and (II) a VL domain comprising at least one, at least two, or all three VL CDR sequences selected from (d) LCDR1 comprising the amino acid sequence of SEQ ID NO: 15; (e) LCDR2 comprising the amino acid sequence of SEQ ID NO: 16; and (f) LCDR3 comprising the amino acid sequence of SEQ ID NO: 17.

In some embodiments, the anti-CCR8 antibody comprises (I) a VH domain comprising at least one, at least two, or all three VH CDR sequences selected from (a) HCDR1 comprising the amino acid sequence of SEQ ID NO: 24; (b) HCDR2 comprising the amino acid sequence of SEQ ID NO: 25; (c) HCDR3 comprising the amino acid sequence of SEQ ID NO: 26; and (II) a VL domain comprising at least one, at least two, or all three VL CDR sequences selected from (d) LCDR1 comprising the amino acid sequence of SEQ ID NO: 27; (e) LCDR2 comprising the amino acid sequence of SEQ ID NO: 28; and (f) LCDR3 comprising the amino acid sequence of SEQ ID NO: 29.

In some embodiments, the anti-CCR8 antibody comprises (I) a VH domain comprising at least one, at least two, or all three VH CDR sequences selected from (a) HCDR1 comprising the amino acid sequence of SEQ ID NO: 36; (b) HCDR2 comprising the amino acid sequence of SEQ ID NO: 37; (c) HCDR3 comprising the amino acid sequence of SEQ ID NO: 38; and (II) a VL domain comprising at least one, at least two, or all three VL CDR sequences selected from (d) LCDR1 comprising the amino acid sequence of SEQ ID NO: 39; (e) LCDR2 comprising the amino acid sequence of SEQ ID NO: 40; and (f) LCDR3 comprising the amino acid sequence of SEQ ID NO: 41.

In some embodiments, the anti-CCR8 antibody comprises (I) a VH domain comprising at least one, at least two, or all three VH CDR sequences selected from (a) HCDR1 comprising the amino acid sequence of SEQ ID NO: 48; (b) HCDR2 comprising the amino acid sequence of SEQ ID NO: 49; (c) HCDR3 comprising the amino acid sequence of SEQ ID NO: 50; and (II) a VL domain comprising at least one, at least two, or all three VL CDR sequences selected from (d) LCDR1 comprising the amino acid sequence of SEQ ID NO: 51; (e) LCDR2 comprising the amino acid sequence of SEQ ID NO: 52; and (f) LCDR3 comprising the amino acid sequence of SEQ ID NO: 53.

In some embodiments, the anti-CCR8 antibody comprises (I) a VH domain comprising at least one, at least two, or all three VH CDR sequences selected from (a) HCDR1 comprising the amino acid sequence of SEQ ID NO: 60; (b) HCDR2 comprising the amino acid sequence of SEQ ID NO: 61, 72, or 78; (c) HCDR3 comprising the amino acid sequence of SEQ ID NO: 62, 73, or 79; and (II) a VL domain comprising at least one, at least two, or all three VL CDR sequences selected from (d) LCDR1 comprising the amino acid sequence of SEQ ID NO: 63; (e) LCDR2 comprising the amino acid sequence of SEQ ID NO: 64; and (f) LCDR3 comprising the amino acid sequence of SEQ ID NO: 65.

In some embodiments, the anti-CCR8 antibody comprises (I) a VH domain comprising at least one, at least two, or all three VH CDR sequences selected from (a) HCDR1 comprising the amino acid sequence of SEQ ID NO: 84 or 100; (b) HCDR2 comprising the amino acid sequence of SEQ ID NO: 85; (c) HCDR3 comprising the amino acid sequence of SEQ ID NO: 86; and (II) a VL domain comprising at least one, at least two, or all three VL CDR sequences selected from (d) LCDR1 comprising the amino acid sequence of SEQ ID NO: 87; (e) LCDR2 comprising the amino acid sequence of SEQ ID NO: 88; and (f) LCDR3 comprising the amino acid sequence of SEQ ID NO: 89.

In some embodiments, an anti-CCR8 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 68 or 74. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, but an anti-CCR8 antibody comprising that sequence retains the ability to bind to CCR8. In some embodiments, a total of 1 to 10 amino acids (e.g., 1, 2, 3, 4, 5, 6, 7, 8, 9, or 10 amino acids) have been substituted, inserted and/or deleted in SEQ ID NO: 68 or 74. In some embodiments, substitutions, insertions, or deletions occur in regions outside the CDRs (that is, in the FRs). Optionally, the anti-CCR8 antibody comprises the VH sequence in SEQ ID NO: 68 or 74, including post-translational modifications of that sequence.

In some embodiments, the VH comprises: (a) HCDR1 comprising the amino acid sequence of SEQ ID NO: 60; (b) HCDR2 comprising the amino acid sequence of SEQ ID NO: 61, 72, or 78; (c) HCDR3 comprising the amino acid sequence of SEQ ID NO: 62, 73, or 79.

In some embodiments, an anti-CCR8 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 92 or 96. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, but an anti-CCR8 antibody comprising that sequence retains the ability to bind to CCR8. In some embodiments, a total of 1 to 10 amino acids (e.g., 1, 2, 3, 4, 5, 6, 7, 8, 9, or 10 amino acids) have been substituted, inserted and/or deleted in SEQ ID NO: 92 or 96. In some embodiments, substitutions, insertions, or deletions occur in regions outside the CDRs (that is, in the FRs). Optionally, the anti-CCR8 antibody comprises the VH sequence in SEQ ID NO: 92 or 96, including post-translational modifications of that sequence.

In some embodiments, the VH comprises: (a) HCDR1 comprising the amino acid sequence of SEQ ID NO: 84 or 100; (b) HCDR2 comprising the amino acid sequence of SEQ ID NO: 85; (c) HCDR3 comprising the amino acid sequence of SEQ ID NO: 86.

In some embodiments, an anti-CCR8 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 69 or 75. In some embodiments, a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-CCR8 antibody comprising that sequence retains the ability to bind to CCR8. In some embodiments, a total of 1 to 10 amino acids (e.g., 1, 2, 3, 4, 5, 6, 7, 8, 9, or 10 amino acids) have been substituted, inserted and/or deleted in SEQ ID NO: 69 or 75. In some embodiments, the substitutions, insertions, or deletions occur in regions outside the CDRs (that is, in the FRs). Optionally, the anti-CCR8 antibody comprises the VL sequence in SEQ ID NO: 69 or 75, including post-translational modifications of that sequence.

In some embodiments, the VL comprises: (a) LCDR1 comprising the amino acid sequence of SEQ ID NO: 63; (b) LCDR2 comprising the amino acid sequence of SEQ ID NO: 64; and (c) LCDR3 comprising the amino acid sequence of SEQ ID NO: 65.

In some embodiments, an anti-CCR8 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 93 or 97. In some embodiments, a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-CCR8 antibody comprising that sequence retains the ability to bind to CCR8. In some embodiments, a total of 1 to 10 amino acids (e.g., 1, 2, 3, 4, 5, 6, 7, 8, 9, or 10 amino acids) have been substituted, inserted and/or deleted in SEQ ID NO: 93 or 97. In some embodiments, the substitutions, insertions, or deletions occur in regions outside the CDRs (that is, in the FRs). Optionally, the anti-CCR8 antibody comprises the VL sequence in SEQ ID NO: 93 or 97, including post-translational modifications of that sequence.

In some embodiments, the VL comprises: (a) LCDR1 comprising the amino acid sequence of SEQ ID NO: 87; (b) LCDR2 comprising the amino acid sequence of SEQ ID NO: 88; and (c) LCDR3 comprising the amino acid sequence of SEQ ID NO: 89.

In some embodiments, an anti-CCR8 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 68 or 74 and a light chain variable domain (VL) having at least 90%, 91%, 92%, 93%, 94%, 95%, 96%, 97%, 98%, 99%, or 100% sequence identity to the amino acid sequence of SEQ ID NO: 69 or 75. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, and a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-CCR8 antibody comprising that sequence retains the ability to bind to CCR8. In some embodiments, a total of 1 to 10 amino acids (e.g., 1, 2, 3, 4, 5, 6, 7, 8, 9, or 10 amino acids) have been substituted, inserted and/or deleted in SEQ ID NO: 68 or 74. In some embodiments, a total of 1 to 10 amino acids (e.g., 1, 2, 3, 4, 5, 6, 7, 8, 9, or 10 amino acids) have been substituted, inserted and/or deleted in SEQ ID NO: 69 or 75. In some embodiments, substitutions, insertions, or deletions occur in regions outside the CDRs (that is, in the FRs). In some embodiments, the anti-CCR8 antibody comprises (a) HCDR1 comprising the amino acid sequence of SEQ ID NO: 60; (b) HCDR2 comprising the amino acid sequence of SEQ ID NO: 61, 72, or 78; (c) HCDR3 comprising the amino acid sequence of SEQ ID NO: 62, 73, or 79; (d) LCDR1 comprising the amino acid sequence of SEQ ID NO: 63; (e) LCDR2 comprising the amino acid sequence of SEQ ID NO: 64; and (f) LCDR3 comprising the amino acid sequence of SEQ ID NO: 65.

In some embodiments, the anti-CCR8 antibody comprises the VH sequence in SEQ ID NO: 68 or 74, including post-translational modifications of one or both sequences, and comprises the VL sequence in SEQ ID NO: 69 or 75, including post-translational modifications of one or both sequences.

In some embodiments, an anti-CCR8 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 92 or 96 and a light chain variable domain (VL) having at least 90%, 91%, 92%, 93%, 94%, 95%, 96%, 97%, 98%, 99%, or 100% sequence identity to the amino acid sequence of SEQ ID NO: 93 or 97. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, and a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-CCR8 antibody comprising that sequence retains the ability to bind to CCR8. In some embodiments, a total of 1 to 10 amino acids (e.g., 1, 2, 3, 4, 5, 6, 7, 8, 9, or 10 amino acids) have been substituted, inserted and/or deleted in SEQ ID NO: 92 or 96. In some embodiments, a total of 1 to 10 amino acids (e.g., 1, 2, 3, 4, 5, 6, 7, 8, 9, or 10 amino acids) have been substituted, inserted and/or deleted in SEQ ID NO: 93 or 97. In some embodiments, substitutions, insertions, or deletions occur in regions outside the CDRs (that is, in the FRs). In some embodiments, the anti-CCR8 antibody comprises (a) HCDR1 comprising the amino acid sequence of SEQ ID NO: 84 or 100; (b) HCDR2 comprising the amino acid sequence of SEQ ID NO: 85; (c) HCDR3 comprising the amino acid sequence of SEQ ID NO: 86; (d) LCDR1 comprising the amino acid sequence of SEQ ID NO: 87; (e) LCDR2 comprising the amino acid sequence of SEQ ID NO: 88; and (f) LCDR3 comprising the amino acid sequence of SEQ ID NO: 89.

In some embodiments, the anti-CCR8 antibody comprises the VH sequence in SEQ ID NO: 92 or 96, including post-translational modifications of one or both sequences, and comprises the VL sequence in SEQ ID NO: 93 or 97, including post-translational modifications of one or both sequences.

In some embodiments, an anti-CCR8 antibody comprises a VH as in any of the embodiments provided herein, and a VL as in any of the embodiments provided herein. In some embodiments, the antibody comprises the VH and VL sequences in SEQ ID NO: 68 or 74 and SEQ ID NO: 69 or 75, respectively, including post-translational modifications of those sequences. In some embodiments, the antibody comprises the VH and VL sequences in SEQ ID NO: 92 or 96 and SEQ ID NO: 93 or 97, respectively, including post-translational modifications of those sequences.

In some embodiments, an anti-CCR8 antibody is used in the methods provided herein, wherein the antibody comprises a heavy chain (HC) having at least 90%, 91%, 92%, 93%, 94%, 95%, 96%, 97%, 98%, 99%, or 100% sequence identity to the amino acid sequence of SEQ ID NO: 70 or 76. Optionally, the anti-CCR8 antibody comprises the HC sequence in SEQ ID NO: 70 or 76, including post-translational modifications.

In some embodiments, an anti-CCR8 antibody is used in the methods provided herein, wherein the antibody comprises a HC having at least 90%, 91%, 92%, 93%, 94%, 95%, 96%, 97%, 98%, 99%, or 100% sequence identity to the amino acid sequence of SEQ ID NO: 94 or 98. Optionally, the anti-CCR8 antibody comprises the HC sequence in SEQ ID NO: 94 or 98, including post-translational modifications.

In some embodiments, an anti-CCR8 antibody is used in the methods provided herein, wherein the antibody comprises a light chain (LC) having at least 90%, 91%, 92%, 93%, 94%, 95%, 96%, 97%, 98%, 99%, or 100% sequence identity to the amino acid sequence of SEQ ID NO: 71 or 77. Optionally, the anti-CCR8 antibody comprises the LC sequence in SEQ ID NO: 71 or 77, including post-translational modifications.

In some embodiments, an anti-CCR8 antibody is used in the methods provided herein, wherein the antibody comprises a LC having at least 90%, 91%, 92%, 93%, 94%, 95%, 96%, 97%, 98%, 99%, or 100% sequence identity to the amino acid sequence of SEQ ID NO: 95 or 99. Optionally, the anti-CCR8 antibody comprises the LC sequence in SEQ ID NO: 95 or 99, including post-translational modifications.

In some embodiments, an anti-CCR8 antibody comprises a HC as in any of the embodiments provided herein, and a LC as in any of the embodiments provided herein. In some embodiments, the antibody comprises the HC and LC sequences in SEQ ID NO: 70 or 76 and SEQ ID NO: 71 or 77, respectively, including post-translational modifications of those sequences. In some embodiments, the antibody comprises the HC and LC sequences in SEQ ID NO: 94 or 98 and SEQ ID NO: 95 or 99, respectively, including post-translational modifications of those sequences.

In some embodiments, antibodies that compete with the anti-CCR8 antibodies described herein for binding to CCR8 are used in the methods provided herein. In some embodiments, antibodies compete with the anti-CCR8 antibodies provided herein for binding to an epitope on CCR8.

In some embodiments, competition assays may be used to identify a monoclonal antibody that competes with an anti-CCR8 antibody described herein (such as 1-K16, 1-K17, 6-B09, 7-B16, 13-E16, and/or 19-007) for binding to CCR8. Competition assays can be used to determine whether two antibodies bind the same epitope by recognizing identical or sterically overlapping epitopes or one antibody competitively inhibits binding of another antibody to the antigen. In some embodiments, such a competing antibody binds to the same epitope that is bound by an antibody described herein. Exemplary competition assays include, but are not limited to, routine assays such as those provided in Harlow and Lane (1988) Antibodies: A Laboratory Manual ch.14 (Cold Spring Harbor Laboratory, Cold Spring Harbor, N.Y.). Detailed exemplary methods for mapping an epitope to which an antibody binds are provided in Morris (1996) “Epitope Mapping Protocols,” in Methods in Molecular Biology vol. 66 (Humana Press, Totowa, N.J.). In some embodiments, two antibodies are said to bind to the same epitope if each blocks binding of the other by 50% or more. In some embodiments, the antibody that competes with an anti-CCR8 antibody described herein is a chimeric, humanized, or human antibody. In some embodiments, an antibody that competes with a chimeric, humanized, or human anti-CCR8 antibody as described herein is provided.

Additionally, the present disclosure also includes variants of the foregoing disclosed antibodies, such as variants of a 7-B16 antibody. For example, in some embodiments, the disclosure includes an isolated antibody that binds human CCR8, wherein the antibody comprises a HCDR3 comprising SEQ ID NO: 86 or a variant of SEQ ID NO: 86 comprising 1, 2, or 3 mutations, and wherein the antibody binds to human CCR8 and possesses ADCC activity. In some embodiments, the mutation is a substitution (e.g., a conservative or a non-conservative substitution), a deletion, or an insertion. In some embodiments, the 1, 2, or 3 mutations are located in at least one of amino acid positions 1-4, 6, 7, or 12 of SEQ ID NO: 86. In some embodiments, the substitution is a conservative substitution. In some embodiments, the conservative subsition is at amino acid position 1, 4, or 12 of SEQ ID NO: 86. In some embodiments, the substitution is a non-conservative substitution. In some embodiments, the non-conservative substitution is at amino acid position 7 of SEQ ID NO: 86. In some embodiments, the antibody comprises at least 2 substitions in HCDR3. In some embodiments, the at least 2 substitutions are located in at least one of amino acid positions 1-4, 6, 7, or 12 of SEQ ID NO: 86. In some embodiments, the at least 2 substitutions are conservative substitutions. In some embodiments, at least one of the conservative substitutions are at amino acid position 1, 4, or 12 of SEQ ID NO: 86. In some embodiments, the at least 2 substitutions are non-conservative substitutions. In some embodiments, at least one of the non-conservative substitutions is at amino acid position 7 of SEQ ID NO: 86. In some embodiments, the mutations comprise a conservative substitution and a non-conservative substitution when more than one substitution mutation is present. In some embodiments, the disclosure provides an isolated antibody that binds human CCR8, wherein the antibody comprises a HCDR3 that is at least 90%, 91%, 92%, 93%, 94%, 95%, 96%, 97%, 98%, or 99% identical to SEQ ID NO: 86, and wherein the antibody binds to human CCR8 and possesses ADCC activity. In some embodiments, the HCDR3 comprises an amino acid sequence selected from SEQ ID NO: 86 and any one of SEQ ID NOs: 104-119. In some embodiments, the antibody comprises a HCDR1 comprising SEQ ID NO: 84 or SEQ ID NO: 123. In some embodiments, the antibody comprises a HCDR2 comprising SEQ ID NO: 85 or SEQ ID NO: 124. In some embodiments, the antibody comprises a LCDR1 comprising SEQ ID NO: 87 or SEQ ID NO: 120. In some embodiments, the antibody comprises a LCDR2 comprising SEQ ID NO: 88 or SEQ ID NO: 121. In some embodiments, the antibody comprises a LCDR3 comprising SEQ ID NO: 89 or SEQ ID NO: 122. In some embodiments, the ADCC activity comprises an EC50 value of less than 200, 175, 150, 125, 100, 75, 50, 25, 20, 15, 10, 9, 8, 7, 6, 5, 4, 3, 2, or 1 ng/ml as measured by an ADCC reporter mechanism of action (MOA)-based bioassay. In some embodiments, the ADCC activity is more potent than a 7-B16 antibody. In some embodiments, the ADCC activity is at least as potent as a 7-B16 antibody. In some embodiments, the antibody possesses a K_{D} for human CCR8 that is equal to or lower than a 7-B16 antibody (as determined by Kinetic Exclusion Assay (i.e., KinExA), for example). In some embodiments, the antibody possesses an on-cell K_{D} for human CCR8 that is equal to or lower than a 7-B16 antibody (as determined by Kinetic Exclusion Assay (i.e., KinExA), for example). In some embodiments, the antibody comprises at least one modification that enhances cell killing. In some embodiments, the enhanced cell killing is enhanced antibody-dependent cellular cytotoxicity (ADCC) and/or complement-dependent cytotoxicity (CDC). In some embodiments, the at least one modification is afucosylation. In some embodiments, the at least one modification is one or more heavy chain constant regions mutations at one or more positions selected from L234, L235, G236, S239, F243, H268, D270, R292, S298, Y300, V305, K326, A330, 1332, E333, K334, and P396. In some embodiments, the one or more heavy chain constant region mutations are one or more mutation selected from S239D, S239M, F243L, H268D, D270E, R292P, S298A, Y300L, V305I, K326D, A330L, A330M, 1332E, E333A, K334A, K334E, and P396L. In some embodiments, the one or more heavy chain constant region mutations are selected from: F243L/R292P/Y300L/V305I/P396L, S239D/1332E, S239D/1332E/A330L, S298A/E333A/K334A, L234Y/L235Q/G236W/S239M/H268D/D270E/S298A, and D270E/K326D/A330M/K334E. In some embodiments, the at least one modification is galactosylation. In some embodiments, the antibody binds human CCR8 with an affinity (K_{D}) (as determined by Kinetic Exclusion Assay (i.e., KinExA), for example) of less than 10 nM, or less than 5 nM, or less than 1 nM, or less than 500 pM, or less than 250 pM, or less than 100 pM, or less than 75 pM, or less than 50 pM, or less than 25 pM. In some embodiments, the antibody binds human CCR8 with an on-cell affinity (K_{D}) of less than 10 nM, or less than 5 nM, or less than 1 nM, or less than 500 pM, or less than 250 pM, or less than 100 pM, or less than 75, or less than 50 pM, or less than 25 pM, as determined by, for example, Kinetic Exclusion Assay (i.e., KinExA). In some embodiments, the antibody is a monoclonal antibody. In some embodiments, the antibody is a human or humanized antibody. In some embodiments, the antibody is a full-length antibody. In some embodiments, the antibody is an IgG1 or IgG3 antibody. Such variants can be utilized in methods of treating cancer, including both blood cancers and solid tumors.

In some embodiments, antibodies that bind to any one or more of the epitopes that the antibodies described herein can be used in the methods provided herein. In some embodiments, antibodies that bind and overlap an epitope to which the present antibodies bind to are described. In some embodiments, an antibody is used that competes with at least one of the antibodies described herein. In some embodiments, an antibody is used that competes with at least two of the antibodies described herein. In some embodiments, an antibody is used that competes with at least three of the antibodies described herein. In some embodiments, the entire epitope is bound and/or obstructed by the competing antibody. In some embodiments, a part of the epitope is bound and/or obstructed by the competing antibody. In some embodiments, the competing antibody's paratope binds to at least a part of the epitope of an antibody provided herein. In some embodiments, the competing antibody's paratope binds the target, and a different section of the competing antibody's structure obstruct at least a part of the epitope of an antibody provided herein.

### Exemplary Chimeric Anti-CCR8 Antibodies

In some embodiments, an anti-CCR8 antibody that can be used in the methods described herein is a chimeric antibody. Certain chimeric antibodies are described, for example, in U.S. Pat. No. 4,816,567; and Morrison et al., (1984) Proc. Natl. Acad. Sci. USA, 81:6851-6855 (1984)). In one example, a chimeric antibody comprises a non-human variable region (for example, a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

Nonlimiting exemplary chimeric antibodies include chimeric antibodies comprising the heavy and/or light chain variable regions of an antibody selected from, e.g., antibody 1-K16, 1-K17, 6-B09, 7-B16, 13-E16 and 19-007, as disclosed herein. Additional nonlimiting exemplary chimeric antibodies include chimeric antibodies comprising heavy chain CDR1, CDR2, and CDR3, and/or light chain CDR1, CDR2, and CDR3 of an antibody selected from antibody 1-K16, 1-K17, 6-B09, 7-B16, 13-E16 and 19-O07, as disclosed herein. In some embodiments, the chimeric anti-CCR8 antibody comprises the variable regions described above and binds to CCR8. In some embodiments, the chimeric anti-CCR8 antibody comprises the variable regions described above, binds to CCR8 and inhibits binding of CCR8 to CCL1. In some embodiments, the anti-CCR8 antibody comprises the variable regions described above, binds to CCR8 and enhances an immune response in a subject, and/or increases activation of T cells in a subject following administration of the antibody to the subject. In some embodiments, administration of the anti-CCR8 antibodies described herein stimulates the activity of an immune cell, reduces the downmodulation of an immune cell, or increases a T cell response in a subject.

In some embodiments, a chimeric antibody described herein comprises one or more human constant regions. In some embodiments, the human heavy chain constant region is of an isotype selected from IgA, IgG, IgD, and IgE. In some embodiments, the human light chain constant region is of an isotype selected from κ and λ. In some embodiments, a chimeric antibody described herein comprises a human IgG constant region. In some embodiments, a chimeric antibody described herein comprises a human IgG4 heavy chain constant region. In some embodiments, a chimeric antibody described herein comprises a human IgG4 constant region and a human K light chain.

As noted above, whether or not effector function is desirable may depend on the particular method of treatment intended for an antibody. Thus, in some embodiments, when effector function is desirable, a chimeric anti-CCR8 antibody comprising a human IgG1 heavy chain constant region or a human IgG3 heavy chain constant region is selected. In some embodiments, when effector function is not desirable, a chimeric anti-CCR8 antibody comprising a human IgG4 or IgG2 heavy chain constant region is selected. In some embodiments, enhanced effector function is desirable.

### Exemplary Humanized Anti-CCR8 Antibodies

In some embodiments, humanized antibodies that bind CCR8 can be used in the methods provided herein. Humanized antibodies are useful as therapeutic molecules because humanized antibodies reduce or eliminate the human immune response as compared to non-human antibodies, which can result in an immune response to an antibody therapeutic (such as the human anti-mouse antibody (HAMA) response), and decreased effectiveness of the therapeutic.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (for example, the antibody from which the CDR residues are derived), for example, to restore or improve antibody specificity or affinity.

Humanized antibodies and methods of making them are reviewed, for example, in Almagro and Fransson, (2008) Front. Biosci. 13: 1619-1633, and are further described, for example, in Riechmann et al., (1988) Nature 332:323-329; Queen et al., (1989) Proc. Natl Acad. Sci. USA 86: 10029-10033; U.S. Pat. Nos. 5,821,337, 7,527,791, 6,982,321, and 7,087,409; Kashmiri et al., (2005) Methods 36:25-34; Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling).

Human framework regions that can be used for humanization include but are not limited to: framework regions selected using the “best-fit” method (see, for example, Sims et al. (1993) J. Immunol. 151:2296); framework regions derived from the consensus sequence of human antibodies of a particular subgroup of light or heavy chain variable regions (see, for example, Carter et al. (1992) Proc. Natl. Acad. Sci. USA, 89:4285; and Presta et al. (1993) J. Immunol, 151:2623); human mature (somatically mutated) framework regions or human germline framework regions (see, for example, Almagro and Fransson, (2008) Front. Biosci. 13:1619-1633); and framework regions derived from screening FR libraries (see, for example, Baca et al., (1997) J. Biol. Chem. 272: 10678-10684 and Rosok et al., (1996) J. Biol. Chem. 271:22611-22618).

In some embodiments, a humanized anti-CCR8 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 68 or 74 and a light chain variable domain (VL) having at least 90%, 91%, 92%, 93%, 94%, 95%, 96%, 97%, 98%, 99%, or 100% sequence identity to the amino acid sequence of SEQ ID NO: 69 or 75. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, and a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-CCR8 antibody comprising that sequence retains the ability to bind to CCR8. In some embodiments, a total of 1 to 10 amino acids (e.g., 1, 2, 3, 4, 5, 6, 7, 8, 9, or 10 amino acids) have been substituted, inserted and/or deleted in SEQ ID NO: 68 or 74. In some embodiments, a total of 1 to 10 amino acids (e.g., 1, 2, 3, 4, 5, 6, 7, 8, 9, or 10 amino acids) have been substituted, inserted and/or deleted in SEQ ID NO: 69 or 75. In some embodiments, substitutions, insertions, or deletions occur in regions outside the CDRs (that is, in the FRs). In some embodiments, the anti-CCR8 antibody comprises (a) HCDR1 comprising the amino acid sequence of SEQ ID NO: 60; (b) HCDR2 comprising the amino acid sequence of SEQ ID NO: 61, 72, or 78; (c) HCDR3 comprising the amino acid sequence of SEQ ID NO: 62, 73, or 79; (d) LCDR1 comprising the amino acid sequence of SEQ ID NO: 63; (e) LCDR2 comprising the amino acid sequence of SEQ ID NO: 64; and (f) LCDR3 comprising the amino acid sequence of SEQ ID NO: 65. In some embodiments, the antibody possesses ADCC activity. In some embodiments, the ADCC activity comprises an EC50 value of less than 200, 175, 150, 125, 100, 75, 50, 25, 20, 15, 10, 9, 8, 7, 6, 5, 4, 3, 2, or 1 ng/ml as measured by an ADCC reporter mechanism of action (MOA)-based bioassay. In some embodiments, the ADCC activity is more potent than a 7-B16 antibody. In some embodiments, the ADCC activity is at least as potent as a 7-B16 antibody. In some embodiments, the antibody possesses a K_{D} for human CCR8 that is equal to or lower than a 7-B16 antibody (as determined by Kinetic Exclusion Assay (i.e., KinExA), for example). In some embodiments, the antibody possesses an on-cell K_{D} for human CCR8 that is equal to or lower than a 7-B16 antibody (as determined by Kinetic Exclusion Assay (i.e., KinExA), for example).

In some embodiments, the humanized anti-CCR8 antibody comprises the VH sequence in SEQ ID NO: 68 or 74, including post-translational modifications of one or both sequences, and comprises the VL sequence in SEQ ID NO: 69 or 75, including post-translational modifications of one or both sequences.

In some embodiments, a humanized anti-CCR8 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 92 or 96 and a light chain variable domain (VL) having at least 90%, 91%, 92%, 93%, 94%, 95%, 96%, 97%, 98%, 99%, or 100% sequence identity to the amino acid sequence of SEQ ID NO: 93 or 97. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, and a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-CCR8 antibody comprising that sequence retains the ability to bind to CCR8. In some embodiments, a total of 1 to 10 amino acids (e.g., 1, 2, 3, 4, 5, 6, 7, 8, 9, or 10 amino acids) have been substituted, inserted and/or deleted in SEQ ID NO: 92 or 96. In some embodiments, a total of 1 to 10 amino acids (e.g., 1, 2, 3, 4, 5, 6, 7, 8, 9, or 10 amino acids) have been substituted, inserted and/or deleted in SEQ ID NO: 93 or 97. In some embodiments, substitutions, insertions, or deletions occur in regions outside the CDRs (that is, in the FRs). In some embodiments, the anti-CCR8 antibody comprises (a) HCDR1 comprising the amino acid sequence of SEQ ID NO: 84 or 100; (b) HCDR2 comprising the amino acid sequence of SEQ ID NO: 85; (c) HCDR3 comprising the amino acid sequence of SEQ ID NO: 86; (d) LCDR1 comprising the amino acid sequence of SEQ ID NO: 87; (e) LCDR2 comprising the amino acid sequence of SEQ ID NO: 88; and (f) LCDR3 comprising the amino acid sequence of SEQ ID NO: 89.

In some embodiments, the humanized anti-CCR8 antibody comprises the VH sequence in SEQ ID NO: 92 or 96, including post-translational modifications of one or both sequences, and comprises the VL sequence in SEQ ID NO: 93 or 97, including post-translational modifications of one or both sequences.

Exemplary humanized anti-CCR8 antibodies include antibodies that compete for binding to CCR8 with an antibody or fragment thereof described herein. Thus, in some embodiments, a humanized anti-CCR8 antibody is provided that competes for binding to CCR8 with an antibody or fragment thereof selected from antibody 1-K16, 1-K17, 6-B09, 7-B16, 13-E16 and 19-007. In some embodiments, the humanized anti-CCR8 antibody competes for binding to CCR8 with an antibody described herein and inhibits binding of CCR8 to CCL1. In some embodiments, the humanized anti-CCR8 antibody competes for binding to CCR8 with an antibody described herein.

### Exemplary Human Anti-CCR8 Antibodies

In some embodiments, an anti-CCR8 antibody used in a method provided herein is a human antibody. Human antibodies can be produced using various techniques known in the art. Human antibodies are described generally in van Dijk and van de Winkel, (2001) Curr. Opin. Pharmacol. 5:368-374 and Lonberg, (2008) Curr. Opin. Immunol. 20:450-459. In some embodiments, the human antibody is not a naturally occurring antibody. In some embodiments, the human antibody is a monoclonal antibody; thus, in some embodiments, each of the human antibodies in a set can bind to the same epitope on the antigen.

Human antibodies can be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, (2005) Nat. Biotech. 23: 1117-1125. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, for example, by combining with a different human constant region.

Human antibodies can also be made by hybridoma-based methods. Human myeloma and mouse-human heteromyeloma cell lines for the production of human monoclonal antibodies have been described. (See, for example, Kozbor (1984) J. Immunol, 133: 3001; Brodeur et al., Monoclonal Antibody Production Techniques and Applications, pp. 51-63 (Marcel Dekker, Inc., New York, 1987); and Boerner et al, (1991) J. Immunol., 147:86). Human antibodies generated via human B-cell hybridoma technology are also described in Li et al., (2006) Proc. Natl. Acad. Sci. USA, 103:3557-3562. Additional methods include those described, for example, in U.S. Pat. No. 7,189,826 (describing production of monoclonal human IgM antibodies from hybridoma cell lines) and Ni, (2006) Xiandai Mianyixue, 26(4):265-268 (describing human-human hybridomas). Human hybridoma technology (Trioma technology) is also described in Vollmers and Brandlein, (2005) Histology and Histopathology, 20(3):927-937 (2005) and Vollmers and Brandlein, (2005) Methods and Findings in Experimental and Clinical Pharmacology, 27(3): 185-191.

Human antibodies can also be generated by isolating Fv clone variable domain sequences selected from human-derived phage display libraries. Such variable domain sequences may then be combined with a desired human constant domain. Techniques for selecting human antibodies from antibody libraries are described below.

Antibodies may be isolated by screening combinatorial libraries for antibodies with the desired activity or activities. For example, a variety of methods are known in the art for generating phage display libraries and screening such libraries for antibodies possessing the desired binding characteristics. Such methods are reviewed, for example, in Hoogenboom et al. in Methods in Molecular Biology 178: 1-37 (O'Brien et al., ed., Human Press, Totowa, N J, 2001) and further described, for example, in the McCafferty et al, (1990) Nature 348:552-554; Clackson et al, (1991) Nature 352: 624-628; Marks et al, (1992) J. Mol. Biol 222: 581-597; Marks and Bradbury, in Methods in Molecular Biology 248: 161-175 (Lo, ed., Human Press, Totowa, N J, 2003); Sidhu et al, (2004) J. Mol. Biol. 338(2): 299-310; Lee et al., (2004) J. Mol. Biol. 340(5): 1073-1093; Fellouse, (2004) Proc. Natl. Acad. Sci. USA 101(34): 12467-12472; and Lee et al, (2004) J. Immunol. Methods 284(1-2): 119-132 and PCT publication WO 99/10494.

In certain phage display methods, repertoires of V_{H} and V_{L} genes are separately cloned by polymerase chain reaction (PCR) and recombined randomly in phage libraries, which can then be screened for antigen-binding phage as described in Winter et al., (1994) Ann. Rev. Immunol., 12:433-455. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naïve repertoire can be cloned (for example, from human) to provide a single source of antibodies to a wide range of non-self and also self-antigens without any immunization as described by Griffiths et al., (1993) EMBO J 12:725-734. Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter (1992), J. Mol. Biol, 227:381-388. Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

In some embodiments, the human anti-CCR8 antibody binds to CCR8 and inhibits binding of CCR8 to CCL1.

Exemplary human anti-CCR8 antibodies also include antibodies that compete for binding to CCR8 with a human antibody or fragment thereof described herein. Thus, in some embodiments, a human anti-CCR8 antibody is provided that competes for binding to CCR8 with an antibody or fragment thereof selected from antibody 1-K16, 1-K17, 6-B09, 7-B16, 13-E16 and 19-007. In some embodiments, the human anti-CCR8 antibody competes for binding to CCR8 with an antibody described herein and inhibits binding of CCR8 to CCL1.

In some embodiments, a chimeric human anti-CCR8 antibody is provided, where the antibody comprises the variable region from a human antibody that binds CCR8 and the constant region from a different human antibody. In some embodiments, a chimeric human anti-CCR8 antibody, where the antibody comprises the CDRs from a human antibody that binds CCR8 and a framework from a different human antibody is provided. In some embodiments, the antibody is not a naturally occurring human antibody.

In some embodiments, a human anti-CCR8 antibody comprises one or more human constant regions. In some embodiments, the human heavy chain constant region is of an isotype selected from IgA, IgG, IgD, and IgE. In some embodiments, the human light chain constant region is of an isotype selected from κ and λ. In some embodiments, a human antibody described herein comprises a human IgG constant region. In some embodiments, a human antibody described herein comprises a human IgG4 heavy chain constant region. In some embodiments, a human antibody described herein comprises a human IgG4 constant region and a human K light chain.

In some embodiments, when effector function is desirable, a human anti-CCR8 antibody comprising a human IgG1 heavy chain constant region or a human IgG3 heavy chain constant region is selected. In some embodiments, when effector function is not desirable, a human anti-CCR8 antibody comprising a human IgG4 or IgG2 heavy chain constant region is selected.

As noted herein, the term “human antibody” denotes the genus of possible sequences for the antibody construct, rather than a source of the antibody.

### Exemplary Anti-CCR8 Antibody Constant Regions and Fc Regions

In some embodiments, an antibody described herein comprises one or more human constant regions. In some embodiments, the human heavy chain constant region is of an isotype selected from IgA, IgG, IgD, and IgE. In some embodiments, an antibody described herein comprises a human IgG constant region. In some embodiments, when effector function is desirable, an anti-CCR8 antibody comprising a human IgG1 heavy chain constant region or a human IgG3 heavy chain constant region is selected. In some embodiments, the human light chain constant region is of an isotype selected from κ and λ. In some embodiments, an antibody described herein comprises a human IgG1 heavy chain constant region. In some embodiments, an antibody described herein comprises a human IgG1 constant region and a human K light chain.

In some embodiments, a fusion protein described herein comprises one or more human Fc regions. In some embodiments, the Fc region is of an isotype selected from IgA, IgG, IgD, and IgE. In some embodiments, a fusion protein described herein comprises a human Fc region. In some embodiments, when effector function is desirable, a fusion protein comprising a human IgG1 Fc region or a human IgG3 Fc region is selected.

Throughout the present specification and claims unless explicitly stated or known to one skilled in the art, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

As noted above, whether or not effector function is desirable may depend on the particular method of treatment intended for an antibody. Thus, in some embodiments, when effector function is desirable, an anti-CCR8 antibody comprising a human IgG1 heavy chain constant region or a human IgG3 heavy chain constant region is selected.

In some embodiments, an antibody comprises a variant Fc region having at least one amino acid substitution compared to the Fc region of a wild-type IgG Fc region. In some embodiments, the variant Fc region has two or more amino acid substitutions compared to the wild-type Fc region. In some embodiments, the variant Fc region has three or more amino acid substitutions compared to a wild-type Fc region. In some embodiments, the variant Fc region has at least one, two or three or more Fc region amino acid substitutions described herein. In some embodiments, the variant Fc region herein will possess at least about 80% homology with a native sequence Fc region and/or with an Fc region of a parent polypeptide. In some embodiments, the variant Fc region herein will possess at least about 90% homology with a native sequence Fc region and/or with an Fc region of a parent polypeptide. In some embodiments, the variant Fc region herein will possess at least about 95% homology with a native sequence Fc region and/or with an Fc region of a parent polypeptide. In some embodiments, a heavy chain constant region or Fc region lacks the C-terminal lysine (K) residue. In some such embodiments, the heavy chain constant region or Fc region may be referred to as “desK.” In some embodiments, the heavy chain constant region or Fc region lacking the C-terminal lysine is an IgG, such as an IgG1, IgG2, IgG3, or IgG4.

In some embodiments, an antibody or fusion protein provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

The carbohydrate attached to an Fc region may be altered. Native antibodies produced by mammalian cells typically comprise a branched, biantennary oligosaccharide that is generally attached by an N-linkage to Asn297 of the CH2 domain of the Fc region. See, for example, Wright et al. TIBTECH 15:26-32 (1997). The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody or fusion protein may be made in order to create antibody variants with certain improved properties.

In some embodiments, antibody or fusion protein variants are provided having a carbohydrate structure that lacks fucose attached (directly or indirectly) to an Fc region (i.e., afucosylated). For example, the amount of fucose in such variants may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn297 (for example, complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, that is, between positions 294 and 300, due to minor sequence variations in antibodies. Such fucosylation variants may have improved ADCC function. See, for example, US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, for example, Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such variants are described, for example, in WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such variants may have improved CDC function. Such variants are described, for example, in WO 1997/30087 (Patel et al.); WO 1998/58964 (Raju, S.); and WO 1999/22764 (Raju, S.).

Antibody or Fe region variants are provided with Fe mutations that increase ADCC activity. In some embodiments, an antibody or Fc region variant comprises one or more mutations that enhance FcγRIIIa binding and/or decreased FcγRIIIb binding. Nonlimiting exemplary such mutations may be made at one or more amino acid positions selected from L234, L235, G236, S239, F243, H268, D270, R292, S298, Y300, V305, K326, A330, 1332, E333, K334, and P396. Nonlimiting exemplary mutations include L234Y, L235Q, G236W, S239D, S239M, F243L, H268D, D270E, R292P, S298A, Y300L, V305I, K326D, A330L, A330M, 1332E, E333A, K334A, K334E, and P396L. In some embodiments, an antibody or Fc region variant comprises mutations F243L/R292P/Y300L/V305I/P396L. See, e.g., Stavenhagen et al., 2007, Cancer Res. 67:8882-8890. In some embodiments, an antibody or Fc region variant comprises mutations S239D/I332E or S239D/I332E/A330L. See, e.g., Lazar et al., 2006, PNAS USA, 103: 4005-4010. In some embodiments, an antibody or Fc region variant comprises mutations S298A/E333A/K334A. See, e.g., Shields et al., 2001, J. Biol. Chem., 276: 6591-6604. In some embodiments, an antibody or Fc region variant comprises mutations L234Y/L235Q/G236W/S239M/H268D/D270E/S298A or mutations D270E/K326D/A330M/K334E, or one heavy chain constant region or Fc comprises mutations L234Y/L235Q/G236W/S239M/H268D/D270E/S298A and the other heavy chain constant region or Fc comprises mutations D270E/K326D/A330M/K334E. See, e.g., Mimoto et al., 2013, MAbs, 5:229-236.

Antibody and Fc region variants are also provided with amino-terminal leader extensions. For example, one or more amino acid residues of the amino-terminal leader sequence are present at the amino-terminus of any one or more heavy or light chains of an antibody. An exemplary amino-terminal leader extension comprises or consists of three amino acid residues, VHS, present on one or both light chains of an antibody variant.

The in vivo or serum half-life of human FcRn high affinity binding polypeptides can be assayed, for example, in transgenic mice, in humans, or in non-human primates to which the polypeptides with a variant Fc region are administered. See also, for example, Petkova et al. International Immunology 18(12):1759-1769 (2006).

In some embodiments, the antibody or Fc region variant mediates ADCC in the presence of human effector cells more effectively than a parent antibody. In some embodiments, the antibody or Fc region variant is substantially more effective at mediating ADCC in vitro, when the amounts of polypeptide variant and parent antibody or Fc region used in the assay are essentially the same. In some embodiments, the antibody or Fc region variant is substantially more effective at mediating ADCC in vivo, when the amounts of polypeptide variant and parent antibody or Fc region used in the assay are essentially the same. Generally, such variants will be identified using the in vitro ADCC assay as herein disclosed, but other assays or methods for determining ADCC activity, for example in an animal model etc., are contemplated.

### Additional Anti-CCR8 Antibodies

In some embodiments, anti-CCR8 antibodies that can be used in the methods provided herein can include BMS-986340 (Bristol Myers Squibb), LM-108 (LaNova Medicines), S-531011 (Shionogi), FPA157 (Five Prime, Amgen), IPG-7236 (Immunophage Biomedical), ICP-B05 (InnoCare Pharma Tech), SRF-114 (Surface Oncology), HBM1022 (Harbour BioMed), HFB1011 (HiFiBio), BAY-3375968 (Bayer), IO-1 (Oncurious), ZL-1218 (Zai Lab), GB2101 (Genor), or PSB-114 (Sound Biologics).

In some embodiments, anti-CCR8 antibodies that can be used in the methods provided herein are as described in WO2022078277, WO2022081718, WO2022000443, WO2022042690, or WO2022003156.

In some embodiments, anti-CCR8 antibodies that can be used in the methods provided herein can be obtained from the hybridoma having ATCC Accession No. PTA-6940, PTA-6938, or PTA-6939.

In some embodiments, the anti-CCR8 antibody that can be used in the methods provided herein is the HBM1022 antibody as disclosed in Lu et al. HBM1022, a novel anti-CCR8 antibody depletes tumor-infiltrating regulatory T cells via enhanced ADCC activity, mediates potent anti-tumor activity with Keytruda. Journal for ImmunoTherapy of Cancer 2020; 8:doi: 10.1136/jitc-2020-SITC2020.0711

In some embodiments, the anti-CCR8 antibody that can be used in the methods provided herein is the FPA157 antibody as disclosed in Rankin A, Naik E861 Development of FPA157, an anti-CCR8 depleting antibody engineered to preferentially eliminate tumor-infiltrating T regulatory cells. Journal for ImmunoTherapy of Cancer 2020; 8:doi: 10.1136/jitc-2020-SITC2020.0861

In some embodiments, the anti-CCR8 antibody that can be used in the methods provided herein is the SRFl 14 antibody as disclosed in Lake A, Warren M, Das S, et a1726 SRF114 is a fully human, CCR8 selective IgG1 antibody that induces destruction of tumor Tregs through ADCC. Journal for ImmunoTherapy of Cancer 2020; 8:doi: 10.1136/jitc-2020-SITC2020.0726

In some embodiments, the anti-CCR8 antibody that can be used in the methods provided herein is antibody is the anti-CCR8 h1gG1-nonfucosylated BMS-986340 antibody as disclosed in Lan, Ruth, et al. “Highly selective anti-CCR8 antibody-mediated depletion of regulatory T cells leads to potent antitumor activity alone and in combination with anti-PD-1 in preclinical models.” (2020): 6694-6694 and in Bayati F. Mohammadi M, Valadi M. Jamshidi S, Foma A M, Sharif-Paghaleh E. The Therapeutic Potential of Regulatory T Cells: Challenges and Opportunities. Front Immunol. 2021; 11:585819. Published 2021 Jan. 15. doi: 10.3389/fimmu.2020.585819

In some embodiments, the anti-CCR8 antibody that can be used in the methods provided herein is the nanobody as disclosed in Van Damme H, Dombrecht B, Kiss M, Roose H, Allen E, Van Overmeire E, Kancheva D, Martens L, Murgaski A, Bardet P M R, Blancke G, Jans M, Bolli E, Martins M S, Elkrim Y, Dooley J, Boon L, Schwarze J K, Tacke F, Movahedi K, Vandanme N, Neyns B, Ocak S, Scheyltjens I, Vereecke L, Nana F A. Merchiers P, Laoui D, Van Ginderachter J A. Therapeutic depletion of CCR8+ tumor-infiltrating regulatory T cells elicits antitumor immunity and synergizes with anti-PD-1 therapy. J Imnunother Cancer. 2021 Feb.; 9(2):e001749. doi: 10.1136/j itc-2020-001749. PMID: 33589525; PMCID: PMC7887378.

### PD-1 Inhibitors or PD-L1 Inhibitors

The PD-1 inhibitors or PD-L1 inhibitors used in the methods provided herein can be small molecule inhibitors or anti-PD-1 antibodies or anti-PD-L1 antibodies.

Exemplary anti-PD-1 antibodies or anti-PD-L1 antibodies that can be co-administered in the methods provided herein include, for example, pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, and zimberelimab. In some embodiments, the anti-PD-1 antibody is zimberelimab.

Additional illustrative anti-PD-1 antibodies or anti-PD-L1 antibodies that can be co-administered in the methods provided herein include pembrolizumab, nivolumab, cemiplimab, pidilizumab, AMP-224, MEDI0680 (AMP-514), spartalizumab, atezolizumab, avelumab, durvalumab, BMS-936559, cosibelimab (CK-301), sasanlimab (PF-06801591), tislelizumab (BGB-A317), GLS-010 (WBP-3055), AK-103 (HX-008), AK-105, CS-1003, HLX-10, retifanlimab (MGA-012), BI-754091, balstilimab (AGEN-2034), AMG-404, toripalimab (JS-001), cetrelimab (JNJ-63723283), genolimzumab (CBT-501), LZM-009, prolgolimab (BCD-100), lodapolimab (LY-3300054), SHR-1201, camrelizumab (SHR-1210), Sym-021, budigalimab (ABBV-181), PD1-PIK, BAT-1306, avelumab (MSB0010718C), CX-072, CBT-502, dostarlimab (TSR-042), MSB-2311, JTX-4014, BGB-A333, SHR-1316, CS-1001 (WBP-3155, envafolimab (KN-035), sintilimab (IBI-308), HLX-20, KL-A167, STI-A1014, STI-A1015 (IMC-001), BCD-135, FAZ-053, TQB-2450, MDX1105-01, GS-4224, GS-4416, INCB086550, MAX10181, zimberelimab (AB122), spartalizumab (PDR-001), and compounds disclosed in WO2018195321, WO2020014643, WO2019160882, or WO2018195321, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1), RO-7247669 (PD-1/LAG-3), MGD-019 (PD-1/CTLA4), KN-046 (PD-1/CTLA4), MEDI-5752 (CTLA4/PD-1), RO-7121661 (PD-1/TIM-3), RG7769 (PD-1/TIM-3), TAK-252 (PD-1/OX40L), XmAb-20717 (PD-1/CTLA4), AK-104 (CTLA4/PD-1), FS-118 (LAG-3/PD-L1), FPT-155 (CTLA4/PD-L1/CD28), GEN-1046 (PD-L1/4-1BB), bintrafusp alpha (M7824; PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In some embodiments, the anti-PD-1 antibody is selected from the group consisting of zimberelimab (AB122, GLS-010, WBP-3055), pembrolizumab (KEYTRUDA®, MK-3475, SCH900475), nivolumab (OPDIVO®, BMS-936558, MDX-1106), cemiplimab (LIBTAYO®; cemiplimab-rwlc, REGN-2810), pidilizumab (CT-011), AMG-404, MEDI0680 (AMP-514), spartalizumab (PDR001), tislelizumab (BGB-A317), toripalimab (JS-001), genolimzumab (CBT-501, APL-501, GB 226), camrelizumab (SHR-1210), sintilimab (TYVYT®; IBI-308), dostarlimab (TSR-042, WBP-285), sasanlimab (PF-06801591), cetrelimab (JNJ-63723283), serplulimab (HLX-10), retifanlimab (MGA-012), balstilimab (AGEN-2034), prolgolimab (BCD-100), budigalimab (ABBV-181), vopratelimab (JTX-4014), AK-103 (HX-008), AK-105, CS-1003, BI-754091, LZM-009, Sym-021, BAT-1306, PD1-PIK, as well as multi-specific inhibitors tebotelimab (MGD013; PD-1/LAG-3), RG-6139 (RO-7247669 PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1) and MEDI-5752 (CTLA4/PD-1).

In some embodiments, the anti-PD-L1 antibody is selected from the group consisting of atezolizumab (TECENTRIQ®), avelumab (BAVENCIO®; MSB0010718C), envafolimab (ASC22), durvalumab (IMFINZI®; MEDI-4736), cosibelimab (CK-301), lodapolimab (LY 3300054), garivulimab (BGB-A333), envafolimab (KN035), opucolimab (HLX-20), manelimab (BCD-135), CX-072, CBT-502 (TQB-2450), MSB-2311, SHR-1316, sugemalimab (CS-1001; WBP3155), A167 (KL-A167, HBM 9167), STI-A1015 (IMC-001), FAZ-053, BMS-936559 (MDX1105), INCB086550, as well as multi-specific inhibitors GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR).

In some embodiments the small molecule PD-1 inhibitors or PD-L1 inhibitors are selected from the group consisting of CA-170, GS-4224, GS-4416, INCB99280, INCB99318, and lazertinib.

### Chemotherapeutic Agents

In some embodiments, the chemotherapeutic agent that can be co-administered in the methods provided herein is selected from the group consisting of a platinum complex, a taxane, pemetrexed, gemcitabine, fluorouracil, irinotecan, etoposide, and doxorubicin. In some embodiments the platinum complex is selected from the group consisting of carboplatin, cisplatin, and oxaliplatin. In some embodiments the taxane is selected from the group consisting of paclitaxel, albumin paclitaxel (e.g., ABRAXANE®) and docetaxel.

In some embodiments, the chemotherapeutic agent that can be co-administered in the methods provided herein is selected from capectiabine, cyclophosphamide, dacarbazine, temozolomide, cyclophosphamide, docetaxel, doxorubicin, daunorubicin, cisplatin, carboplatin, epirubicin, eribulin, 5-FU, gemcitabine, irinotecan, ixabepilone, methotrexate, mitoxantrone, oxaliplatin, paclitaxel, nab-paclitaxel, ABRAXANE® (protein-bound paclitaxel), pemetrexed, vinorelbine, and vincristine. In some embodiments, the chemotherapeutic agent is a kinase inhibitor. Nonlimiting exemplary kinase inhibitors include erlotinib, afatinib, gefitinib, crizotinib, dabrafenib, trametinib, vemurafenib, and cobimetanib.

### Breast Cancer

In some embodiments the chemotherapeutic agent that can be co-administered in the methods provided herein to a subject having breast cancer is selected from the group consisting of albumin-bound paclitaxel, anastrozole, atezolizumab, capecitabine, carboplatin, cisplatin, cyclophosphamide, docetaxel, doxorubicin, epirubicin, everolimus, exemestane, fluorouracil, fulvestrant, gemcitabine, ixabepilone, lapatinib, letrozole, methotrexate, mitoxantrone, paclitaxel, pegylated liposomal doxorubicin, pertuzumab, tamoxifen, toremifene, trastuzumab, vinorelbine, and any combinations thereof.

### Triple Negative Breast Cancer

In some embodiments the chemotherapeutic agent that can be co-administered in the methods provided herein to a subject having TNBC is selected from the group consisting of cyclophosphamide, docetaxel, doxorubicin, epirubicin, fluorouracil, paclitaxel, and combinations thereof.

### Colorectal Cancer

In some embodiments the chemotherapeutic agent that can be co-administered in the methods provided herein to a subject having colorectal cancer (e.g., MSS mCRC) is selected from the group consisting of capecitabine, cetuximab, fluorouracil, irinotecan, leucovorin, oxaliplatin, panitumumab, ziv-aflibercept, and any combinations thereof.

### Esophageal and Esophagogastric Junction Cancer

In some embodiments the chemotherapeutic agent that can be co-administered in the methods provided herein to a subject having esophageal or esophagogastric junction cancer is selected from the group consisting of capecitabine, carboplatin, cisplatin, docetaxel, epirubicin, fluoropyrimidine, fluorouracil, irinotecan, leucovorin, oxaliplatin, paclitaxel, and any combinations thereof.

### Gastric Cancer

In some embodiments the chemotherapeutic agent that can be co-administered in the methods provided herein to a subject having gastric cancer is selected from the group consisting of capecitabine, carboplatin, cisplatin, docetaxel, epirubicin, fluoropyrimidine, fluorouracil, irinotecan, leucovorin, mitomycin, oxaliplatin, paclitaxel, and any combinations thereof.

### Head and Neck Cancer

In some embodiments the chemotherapeutic agent that can be co-administered in the methods provided herein to a subject having head and neck cancer is selected from the group consisting of afatinib, bleomycin, capecitabine, carboplatin, cetuximab, cisplatin, docetaxel, fluorouracil, gemcitabine, hydroxyurea, methotrexate, nivolumab, paclitaxel, vinorelbine, and any combinations thereof.

### Non-Small Cell Lung Cancer Combination Therapy

In some embodiments the chemotherapeutic agent that can be co-administered in the methods provided herein to a subject having non-small cell lung cancer (NSCLC) is selected from the group consisting ofafatinib, albumin-bound paclitaxel, alectinib, cabozantinib, carboplatin, cisplatin, crizotinib, dabrafenib, docetaxel, erlotinib, etoposide, gemcitabine, paclitaxel, pemetrexed, vandetanib, vemurafenib, vinblastine, vinorelbine, and any combinations thereof.

### Small Cell Lung Cancer

In some embodiments the chemotherapeutic agent that can be co-administered in the methods provided herein to a subject having small cell lung cancer (SCLC) is selected from the group consisting of bendamustime, carboplatin, cisplatin, cyclophosphamide, docetaxel, doxorubicin, etoposide, gemcitabine, irinotecan, paclitaxel, temozolomide, topotecan, vincristine, vinorelbine, and any combinations thereof.

### Ovarian Cancer

In some embodiments the chemotherapeutic agent that can be co-administered in the methods provided herein to a subject having ovarian cancer is selected from the group consisting of 5-flourouracil, albumin bound paclitaxel, altretamine, anastrozole, capecitabine, carboplatin, cisplatin, cyclophosphamide, docetaxel, doxorubicin, etoposide, exemestane, gemcitabine, ifosfamide, irinotecan, letrozole, leuprolide acetate, liposomal doxorubicin, megestrol acetate, melphalan, olaparib, oxaliplatin, paclitaxel, pazopanib, pemetrexed, tamoxifen, topotecan, vinorelbine, and any combinations thereof.

### Routes of Administration

In some embodiments, the anti-CCR8 antibodies, anti-PD-1 antibodies, anti-PD-L1 antibodies, and chemotherapeutic agents described herein can be administered in vivo by various routes, including, but not limited to, intravenous, intra-arterial, parenteral, intratumoral, intraperitoneal or subcutaneous. The appropriate formulation and route of administration may be selected according to the intended application.

### Kits/Articles of Manufacture

Provided herein are also kits, medicines, compositions, and unit dosage forms for use in any of the methods described herein.

Kits can include one or more containers comprising anti-CCR8 antibodies, anti-PD-1 antibodies, anti-PD-L1 antibodies, or chemotherapeutic agents described, or unit dosage forms and/or articles of manufacture. In some embodiments, a unit dosage is provided wherein the unit dosage contains a predetermined amount of a composition comprising an antibody and/or fusion protein provided herein, with or without one or more additional agents. In some embodiments, such a unit dosage is supplied in single-use prefilled syringe for injection. In some embodiments, the composition contained in the unit dosage can comprise saline, sucrose, or the like; a buffer, such as phosphate, or the like; and/or be formulated within a stable and effective pH range. In some embodiments, the composition can be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition comprises heparin and/or a proteoglycan.

In some embodiments, kits further comprise instructions for use in the treatment of cancer in accordance with any of the methods described herein. The kit may further comprise a description of selection an individual suitable or treatment. Instructions supplied in the kits are typically written instructions on a label or package insert (for example, a paper sheet included in the kit), but machine-readable instructions (for example, instructions carried on a magnetic or optical storage disk) are also acceptable. In some embodiments, the kit further comprises another therapeutic agent.

The kits are in suitable packaging. Suitable packaging includes, but is not limited to, vials, bottles, jars, flexible packaging (for example, sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

### EXAMPLES

### Example 1: Syngeneic Mouse Tumor Studies of Anti-CCR8 Antibody/Chemotherapy Combination Treatments

The ability of an anti-CCR8 antibody to augment tumor-specific effector T cell responses when paired with low-dose chemotherapy with or without an anti-PD-1 mAb was tested in vivo in different mouse syngeneic models, including 4T1 (breast cancer), Pan02 (pancreatic cancer), B16F10 (melanoma), LLC (lung cancer). 4T1 and Pan02 models are commonly understood to represent solid tumor models with strong immunosuppressive components. B16F10 and LLC are commonly viewed as cold tumor models.

### Reagents

The anti-CCR8 antibody used for the studies described herein is a mouse IgG2a isotype, and as described, e.g., by Campbell, J. R., et al. (2021). “Fc-Optimized Anti-CCR8 Antibody Depletes Regulatory T Cells in Human Tumor Models.” Cancer Res 81(11): 2983-2994. Isotype Control is a mouse IgG2a (BioXcell). Anti-PD-1 antibody is a mouse IgG1 antibody with D265A mutation.

### 4T1 Model

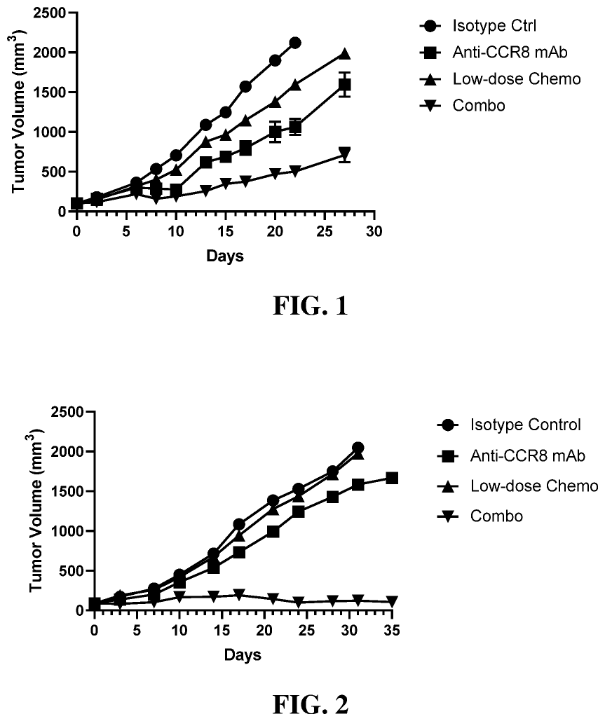

Following inoculation, mice were randomized and grouped (n=8 animals per group) when tumors reached 80-120 mm³ and administered a single dose of low-dose cisplatin at 3 mg/kg (approximately 50% of most efficacious dose) and/or anti-CCR8 antibody Q3D at 1 mg/kg. A control group received an isotype control antibody. Results are shown in FIG. 1. The anti-CCR8 antibody/cisplatin combination treatment was found to result in greater tumor growth inhibition than anti-CCR8 antibody or cisplatin single agent treatments respectively.

### Pan02 Model

Following inoculation, mice were randomized and grouped (n=8 animals per group) when tumors reached 80-120 mm³ and administered a single dose of low dose gemcitabine at 15 mg/kg (approximately 50% of most efficacious dose) and/or anti-CCR8 antibody Q3D at 1 mg/kg. A control group received an isotype control antibody. Results are shown in FIG. 2. The anti-CCR8 antibody/gemcitabine combination treatment was found to result in greater tumor growth inhibition than anti-CCR8 antibody or gemcitabine single agent treatments respectively.

### B16F10 Model

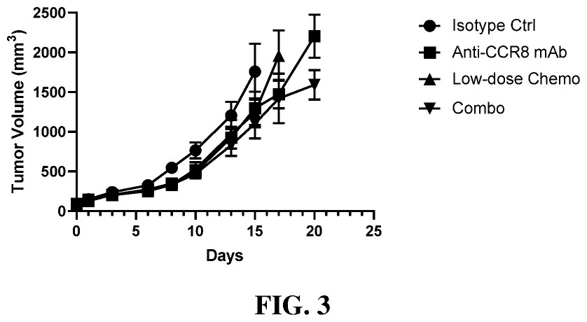

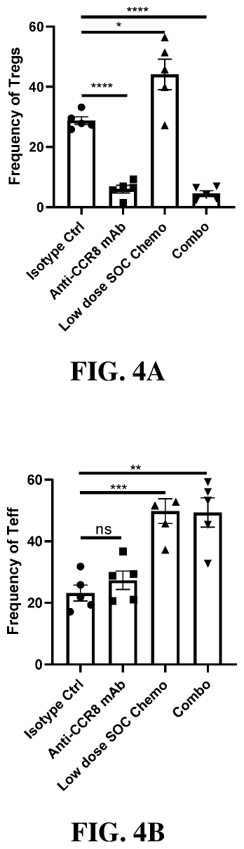

A first study using the B16F10 model tested the relative antitumor activities of anti-CCR8 antibody and gemcitabine administrations as single agents and in combination. Following inoculation, mice were randomized and grouped (n=10 animals per group) when tumors reached 80-120 mm³ and administered a single dose of low dose gemcitabine at 15 mg/kg (approximately 50% of most efficacious dose) and/or anti-CCR8 antibody Q3D at 1 mg/kg. A control group received an isotype control antibody. Results are shown in FIG. 3, FIG. 4A and FIG. 4B. Anti-CCR8 antibody and gemcitabine administrations, alone or in combination, did not result in substantial tumor growth inhibition (FIG. 3). Tumors were collected at Day 5 post-dose. Pharmacodynamic data showed significant Treg depletion in the tumors in the anti-CCR8 antibody treated groups (FIG. 4A), and significant CD8+ T-cell infiltration in the tumors of chemotherapy treated groups (FIG. 4B). Briefly, tumors were dissociated and prepared for flow cytometry staining. Cells were stained as follows: L/D, CD45, CD3, CD4, CD8, CD25, FoxP3. Samples were acquired on flow cytometer and data generated was used to determine Treg frequency (L/D negative, CD45 positive, CD3 positive, CD4 positive, CD25 positive, FoxP3 positive) and CD8 frequency (L/D negative, CD45 positive, CD3 positive, CD8 positive).

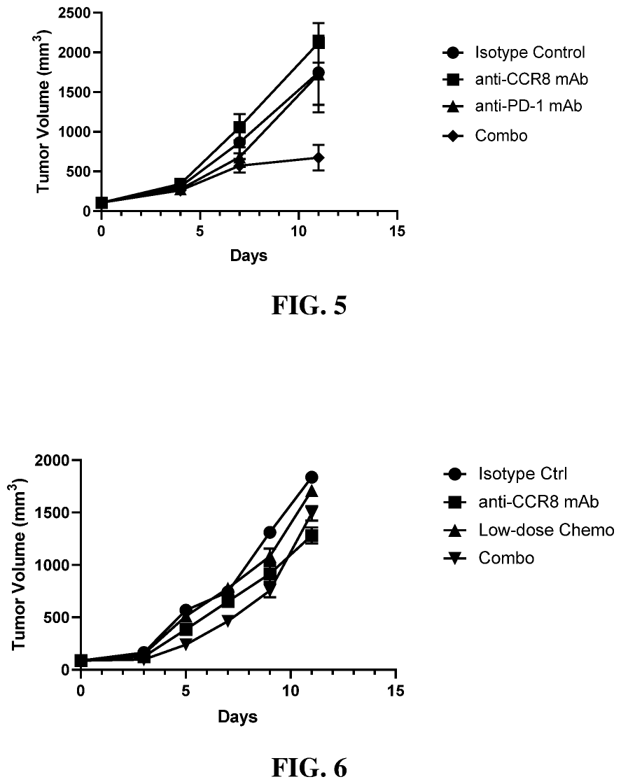

A second study using the B16F10 model tested an anti-CCR8 antibody and an anti-PD-1 antibody either as single agents or in combination. Results are shown in FIG. 5. The anti-CCR8 antibody/anti-PD-1 antibody combination treatment resulted in substantially greater tumor growth inhibition than single agent treatments.

### LLC Model

A first study using the LLC model tested the relative antitumor activities of anti-CCR8 antibody and docetaxel administrations as single agents and in combination. Following inoculation, mice were randomized and grouped (n=8 animals per group) when tumors reached 80-120 mm³ and administered a single dose of low dose docetaxel at 5 mg/kg (approximately 50% of most efficacious dose) and/or anti-CCR8 antibody Q3D at 1 mg/kg. A control group received an isotype control antibody. Results are shown in FIG. 6. Anti-CCR8 antibody and docetaxel administrations, alone or in combination, did not result in substantial tumor growth inhibition (FIG. 6). anti-CCR8 antibody and docetaxel administrations as single agents and in combination.

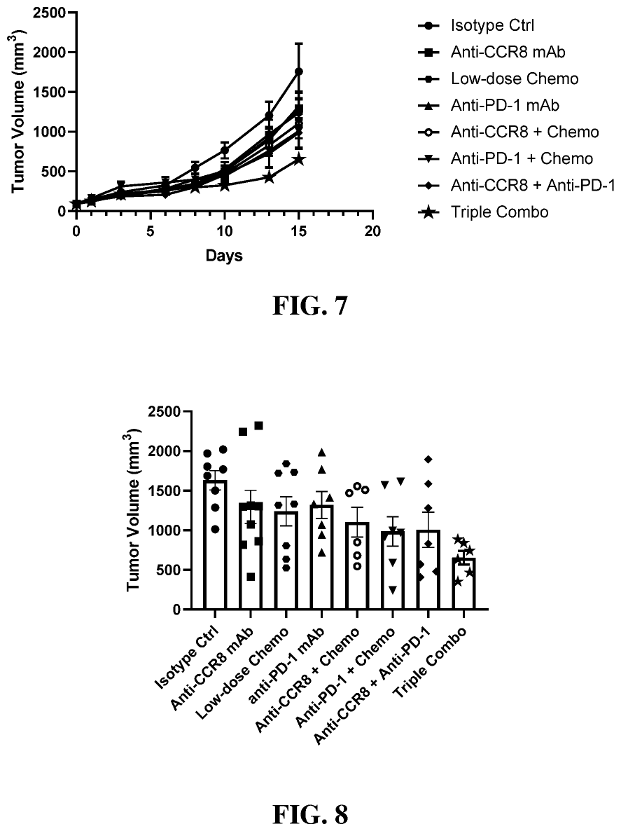

A second study using the LLC model tested the relative antitumor activities of anti-CCR8 antibody, anti-PD-1 antibody, and low dose docetaxel as single agents, dual combinations, and a triple combination. Following inoculation, mice were randomized and grouped (n=10 animals per group) when tumors reached 80-120 mm³ and administered a single dose of docetaxel at 5 mg/kg, and/or anti-CCR8 antibody Q3D at 1 mg/kg, and/or anti-PD-1 antibody Q3D at 10 mg/kg. A control group received an isotype control antibody. Tumor growth inhibition results are shown in FIG. 7. FIG. 8 highlights data for individual mice in different treatment cohorts on day 15. Partial tumor growth inhibition was observed with each anti-CCR8 antibody, anti-PD-1 antibody, and docetaxel single agent treatment. Improved tumor growth inhibition was observed with anti-CCR8 antibody/docetaxel, anti-PD-1 antibody/docetaxel, and anti-CCR8 antibody/anti-PD-1 antibody dual combination treatments. Further improved tumor growth inhibition was observed with an anti-CCR8 antibody/anti-PD-1 antibody/docetaxel triple combination treatment.

### CONCLUSIONS

This Example demonstrates that targeted Treg depletion in combination with chemotherapy can lead to significantly reduced tumor growth in breast and pancreatic tumor models with strong immunosuppressive components (FIG. 1 and FIG. 2). No efficacy of chemotherapy and anti-CCR8 mAb combination was observed in models with low immune infiltration (FIG. 3) despite confirmed depletion of intra-tumoral Tregs (FIG. 4A) and observed Teff infiltration (FIG. 4B). With the addition of PD-1 blockade, selective Treg depletion was found to result in a strong response in a cold tumor model (FIG. 5). Addition of an anti-PD-1 antibody to low-dose chemotherapy and Treg depletion in the LLC model (unresponsive to single therapy or combination therapy anti-CCR8 antibody and chemotherapy, FIG. 6) resulted in 72% tumor growth inhibition in the triple combination group (FIG. 7 and FIG. 8) suggesting PD-1 expression can limit T cell responses even in the absence of Tregs.

In summary, this Example demonstrated that Treg depletion can augment responses to chemotherapy treatment, including low-dose chemotherapy treatments. PD-1 blockade can potentiate the effects of selective Treg depletion and chemotherapy to result in strong anti-tumor immune activity.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

TABLE of
Certain Sequences

| Description | Sequence | SEQ ID NO |
|---|---|---|
| Human | MQIITTALVCLLLAGMWPEDVDSKSMQVPFSRCCFSFAEQEI | 1 |
| CCL1.hFc1 | PLRAILCYRNTSSICSNEGLIFKLKRGKEACALDTVGWVQR | |
| Full length | HRKMLRHCPSKRKSGGSGGGSGDKTHTCPPCPAPELLGGPS | |
| sequence | VFLFPPKPKDTLMISRTPEVTCVVVDVSHEDPEVKFNWYVD | |
| (mature | GVEVHNAKTKPREEQYNSTYRVVSVLTVLHQDWLNGKEY | |
| sequence | KCKVSNKALPAPIEKTISKAKGQPREPQVYTLPPSREEMTKN | |
| amino acids | QVSLTCLVKGFYPSDIAVEWESNGQPENNYKTTPPVLDSDGS | |
| 24-316) | FFLYSKLTVDKSRWQQGNVFSCSVMHEALHNHYTQKSLSLS | |
| | PGKGSGEPEA | |
| Human | MQIITTALVCLLLAGMWPEDVDSKSMQVPFSRCCFSFAEQEI | 2 |
| CCL | PLRAILCYRNTSSICSNEGLIFKLKRGKEACALDTVGWVQR | |
| sequence | HRKMLRHCPSKRK | |
| (mature | | |
| sequence | | |
| amino acids | | |
| 24-96) | | |
| Human | SGGSGGGSG | 3 |
| CCL1.hFc1 | | |
| Linker | | |
| sequence | | |
| hFc1 | DKTHTCPPCPAPELLGGPSVFLFPPKPKDTLMISRTPEVTCVV | 4 |
| sequence | VDVSHEDPEVKFNWYVDGVEVHNAKTKPREEQYNSTYRV | |
| | VSVLTVLHQDWLNGKEYKCKVSNKALPAPIEKTISKAKGQP | |
| | REPQVYTLPPSREEMTKNQVSLTCLVKGFYPSDIAVEWESNG | |
| | QPENNYKTTPPVLDSDGSFFLYSKLTVDKSRWQQGNVFSCS | |
| | VMHEALHNHYTQKSLSLSPGKGSGEPEA | |
| MC148.hFc | LARRKCCLNPTNRPIPNPLLQDLSRVDYQAIGHDCGREAFRV | 5 |
| 1 Full | TLQDGRQGCVSVGNKSLLDWLRGHKDLCPQIWSGCESLGG | |
| length | GSGGGSDKTHTCPPCPAPELLGGPSVFLFPPKPKDTLMISRTP | |
| sequence | EVTCVVVDVSHEDPEVKFNWYVDGVEVHNAKTKPREEQY | |
| | NSTYRVVSVLTVLHQDWLNGKEYKCKVSNKALPAPIEKTIS | |
| | KAKGQPREPQVYTLPPSREEMTKNQVSLTCLVKGFYPSDIAV | |
| | EWESNGQPENNYKTTPPVLDSDGSFFLYSKLTVDKSRWQQG | |
| | NVFSCSVMHEALHNHYTQKSLSLSPGKGSGEPEA | |
| MC148 | LARRKCCLNPTNRPIPNPLLQDLSRVDYQAIGHDCGREAFRV | 6 |
| sequence | TLQDGRQGCVSVGNKSLLDWLRGHKDLCPQIWSGCESL | |
| MC148.hFc | GGGSGGGS | 7 |
| 1 Linker | | |
| sequence | | |
| 1-K16 VH | EVQLVETGGGLVQPKGSLKLSCAASGFTFNINAMNWVRQA | 8 |
| | PGKGLEWVARIRSKSNNYATYYADSVKDRFTISRDDSQSML | |
| | YLQMNNLKTEDTAMYYCVRGGYGSSPYDMDYWGQGTSV | |
| | TVSS | |
| 1-K16 VL | DIVMTQAAPSVPVTPGESVSISCRSSKSLLHSNGNTYLYWFL | 9 |
| | QRPGQSPQLLIYRMSNLASGVPDRFSGSGSGTAFTLRISRVE | |
| | AEDVGVYYCMQHLEYPFTFGAGTKLELK | |
| 1-K16 HC | EVQLVETGGGLVQPKGSLKLSCAASGFTFNINAMNWVRQA | 10 |
| | PGKGLEWVARIRSKSNNYATYYADSVKDRFTISRDDSQSML | |
| | YLQMNNLKTEDTAMYYCVRGGYGSSPYDMDYWGQGTSV | |
| | TVSSAKTTPPSVYPLAPGSAAQTNSMVTLGCLVKGYFPEPV | |
| | TVTWNSGSLSSGVHTFPAVLESDLYTLSSSVTVPSSPRPSETV | |
| | TCNVAHPASSTKVDKKIVPRDCGCKPCICTVPEVSSVFIFPPK | |
| | PKDVLTITLTPKVTCVVVDISKDDPEVQFSWFVDDVEVHTA | |
| | QTQPREEQFNSTFRSVSELPIMHQDWLNGKEFKCRVNSAAF | |
| | PAPIEKTISKTKGRPKAPQVYTIPPPKEQMAKDKVSLTCMIT | |
| | DFFPEDITVEWQWNGQPAENYKNTQPIMNINGSYFVYSKL | |
| | NVQKSNWEAGNTFTCSVLHEGLHNHHTEKSLSHSPG | |
| 1-K16 LC | DIVMTQAAPSVPVTPGESVSISCRSSKSLLHSNGNTYLYWFL | 11 |
| | QRPGQSPQLLIYRMSNLASGVPDRFSGSGSGTAFTLRISRVE | |
| | AEDVGVYYCMQHLEYPFTFGAGTKLELKRADAAPTVSIFPP | |
| | SSEQLTSGGASVVCFLNNFYPKDINVKWKIDGSERQNGVLN | |
| | SWTDQDSKDSTYSMSSTLTLTKDEYERHNSYTCEATHKTST | |
| | SPIVKSFNRNEC | |
| 1-K16 | GFTFNINAMN | 12 |
| CDR-H1 | | |
| 1-K16 | RIRSKSNNYATYYADSV | 13 |
| CDR-H2 | | |
| 1-K16 | VRGGYGSSPYDMDY | 14 |
| CDR-H3 | | |
| 1-K16 | RSSKSLLHSNGNTYLY | 15 |
| CDR-L1 | | |
| 1-K16 | RMSNLAS | 16 |
| CDR-L2 | | |
| 1-K16 | MQHLEYPFT | 17 |
| chimera | | |
| CDR-L3 | | |
| 1-K16 | EVQLVETGGGLVQPKGSLKLSCAASGFTFNINAMNWVRQA | 18 |
| chimera HC | PGKGLEWVARIRSKSNNYATYYADSVKDRFTISRDDSQSML | |
| | YLQMNNLKTEDTAMYYCVRGGYGSSPYDMDYWGQGTSV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPELLGGP | |
| | SVFLFPPKPKDTLMISRTPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSREEMTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPG | |
| 1-K16 | DIVMTQAAPSVPVTPGESVSISCRSSKSLLHSNGNTYLYWFL | 19 |
| chimera LC | QRPGQSPQLLIYRMSNLASGVPDRESGSGSGTAFTLRISRVE | |
| | AEDVGVYYCMQHLEYPFTFGAGTKLELKRTVAAPSVFIFPP | |
| | SDEQLKSGTASVVCLLNNFYPREAKVQWKVDNALQSGNSQ | |
| | ESVTEQDSKDSTYSLSSTLTLSKADYEKHKVYACEVTHQGL | |
| | SSPVTKSFNRGEC | |
| 6-B09 VH | QVQLKESGPGLVAPSQSLSITCTVSGFSLARYDISWIRQPPGK | 20 |
| | GLEWLGVIWTGGGTNYNSAFMSRLSISKDNSKSQVFLKMN | |
| | GLQTDDTAIYYCVSIRYDETYWGQGTLVTVSA | |
| 6-B09 VL | QIVLTQSPAIMSASPGEKVTITCSASSSVIYMHWFQQKPGTSP | 21 |
| | KLWIYATSNLASGVPARFSGSGSGTSYSLTISRMEAEDAATY | |
| | YCQQRSSYPLTFGAGTKLELK | |
| 6-B09 HC | QVQLKESGPGLVAPSQSLSITCTVSGFSLARYDISWIRQPPGK | 22 |
| | GLEWLGVIWTGGGTNYNSAFMSRLSISKDNSKSQVFLKMN | |
| | GLQTDDTAIYYCVSIRYDETYWGQGTLVTVSAAKTTAPSVY | |
| | PLAPVCGDTTGSSVTLGCLVKGYFPEPVTLTWNSGSLSSGV | |
| | HTFPAVLQSDLYTLSSSVTVTSSTWPSQSITCNVAHPASSTKV | |
| | DKKIEPRGPTIKPCPPCKCPAPNLLGGPSVFIFPPKIKDVLMIS | |
| | LSPIVTCVVVDVSEDDPDVQISWFVNNVEVHTAQTQTHRED | |
| | YNSTLRVVSALPIQHQDWMSGKEFKCKVNNKDLPAPIERTIS | |
| | KPKGSVRAPQVYVLPPPEEEMTKKQVTLTCMVTDFMPEDIY | |
| | VEWTNNGKTELNYKNTEPVLDSDGSYFMYSKLRVEKKNW | |
| | VERNSYSCSVVHEGLHNHHTTKSFSRTPG | |
| 6-B09 LC | QIVLTQSPAIMSASPGEKVTITCSASSSVIYMHWFQQKPGTSP | 23 |
| | KLWIYATSNLASGVPARFSGSGSGTSYSLTISRMEAEDAATY | |
| | YCQQRSSYPLTFGAGTKLELKRADAAPTVSIFPPSSEQLTSG | |
| | GASVVCFLNNFYPKDINVKWKIDGSERQNGVLNSWTDQDS | |
| | KDSTYSMSSTLTLTKDEYERHNSYTCEATHKTSTSPIVKSFN | |
| | RNEC | |
| 6-B09 | GFSLARYDIS | 24 |
| CDR-H1 | | |
| 6-B09 | GGTNYNSAFMSRLSIS | 25 |
| CDR-H2 | | |
| 6-B09 | VSIRYDETY | 26 |
| CDR-H3 | | |
| 6-B09 | SASSSVIYMH | 27 |
| CDR-L1 | | |
| 6-B09 | TSNLASG | 28 |
| CDR-L2 | | |
| 6-B09 | QQRSSYPLT | 29 |
| CDR-L3 | | |
| 6-B09 | QVQLKESGPGLVAPSQSLSITCTVSGFSLARYDISWIRQPPGK | 30 |
| chimera HC | GLEWLGVIWTGGGTNYNSAFMSRLSISKDNSKSQVFLKMN | |
| | GLQTDDTAIYYCVSIRYDETYWGQGTLVTVSAASTKGPSVF | |
| | PLAPSSKSTSGGTAALGCLVKDYFPEPVTVSWNSGALTSGV | |
| | HTFPAVLQSSGLYSLSSVVTVPSSSLGTQTYICNVNHKPSNTK | |
| | VDKKVEPKSCDKTHTCPPCPAPELLGGPSVFLFPPKPKDTLM | |
| | ISRTPEVTCVVVDVSHEDPEVKFNWYVDGVEVHNAKTKPR | |
| | EEQYNSTYRVVSVLTVLHQDWLNGKEYKCKVSNKALPAPIE | |
| | KTISKAKGQPREPQVYTLPPSREEMTKNQVSLTCLVKGFYPS | |
| | DIAVEWESNGQPENNYKTTPPVLDSDGSFFLYSKLTVDKSR | |
| | WQQGNVFSCSVMHEALHNHYTQKSLSLSPG | |
| 6-B09 | QIVLTQSPAIMSASPGEKVTITCSASSSVIYMHWFQQKPGTSP | 31 |
| chimera | KLWIYATSNLASGVPARFSGSGSGTSYSLTISRMEAEDAATY | |
| LC | YCQQRSSYPLTFGAGTKLELKRTVAAPSVFIFPPSDEQLKSGT | |
| | ASVVCLLNNFYPREAKVQWKVDNALQSGNSQESVTEQDSK | |
| | DSTYSLSSTLTLSKADYEKHKVYACEVTHQGLSSPVTKSFNR | |
| | GEC | |
| 13-E16 VH | EVQLVETGGGLVQPKGSLKLSCAASGFTFSTNAMNWVRQA | 32 |
| | PGKGLEWIARIRSKSNNYATHYADSVKDRFTISRDDSQSILH | |
| | LQMNNLKNEDTAMYYCVRDSHYYVSTYVGLAWFAYWGQ | |
| | GTLVTVSA | |
| 13-E16 VL | QIVLTQSPAIMSASPGEKVTITCSASSSVSYMHWFQQKPGTS | 33 |
| | PKLWIYSTSNLASGVPARFSGSGSGTSYFLTISRMEAEDAAT | |
| | YYCQQRSSYPYTFGGGTKLERK | |
| 13-E16 HC | EVQLVETGGGLVQPKGSLKLSCAASGFTFSTNAMNWVRQA | 34 |
| | PGKGLEWIARIRSKSNNYATHYADSVKDRFTISRDDSQSILH | |
| | LQMNNLKNEDTAMYYCVRDSHYYVSTYVGLAWFAYWGQ | |
| | GTLVTVSAAKTTAPSVYPLAPVCGDTTGSSVTLGCLVKGYF | |
| | PEPVTLTWNSGSLSSGVHTFPAVLQSDLYTLSSSVTVTSSTW | |
| | PSQSITCNVAHPASSTKVDKKIEPRGPTIKPCPPCKCPAPNLL | |
| | GGPSVFIFPPKIKDVLMISLSPIVTCVVVDVSEDDPDVQISWF | |
| | VNNVEVHTAQTQTHREDYNSTLRVVSALPIQHQDWMSGKE | |
| | FKCKVNNKDLPAPIERTISKPKGSVRAPQVYVLPPPEEEMTK | |
| | KQVTLTCMVTDFMPEDIYVEWTNNGKTELNYKNTEPVLDS | |
| | DGSYFMYSKLRVEKKNWVERNSYSCSVVHEGLHNHHTTKS | |
| | FSRTPG | |
| 13-E16 LC | QIVLTQSPAIMSASPGEKVTITCSASSSVSYMHWFQQKPGTS | 35 |
| | PKLWIYSTSNLASGVPARFSGSGSGTSYFLTISRMEAEDAAT | |
| | YYCQQRSSYPYTFGGGTKLERKRADAAPTVSIFPPSSEQLTS | |
| | GGASVVCFLNNFYPKDINVKWKIDGSERQNGVLNSWTDQD | |
| | SKDSTYSMSSTLTLTKDEYERHNSYTCEATHKTSTSPIVKSF | |
| | NRNEC | |
| 13-E16 | GFTESTNAMN | 36 |
| CDR-H1 | | |
| 13-E16 | RIRSKSNNYATHYADSV | 37 |
| CDR-H2 | | |
| 13-E16 | VRDSHYYVSTYVGLA | 38 |
| CDR-H3 | | |
| 13-E16 | SASSSVSYMH | 39 |
| CDR-L1 | | |
| 13-E16 | TSNLAS | 40 |
| CDR-L2 | | |
| 13-E16 | QQRSSYPYT | 41 |
| CDR-L3 | | |
| 13-E16 | EVQLVETGGGLVQPKGSLKLSCAASGFTFSTNAMNWVRQA | 42 |
| chimera HC | PGKGLEWIARIRSKSNNYATHYADSVKDRFTISRDDSQSILH | |
| | LQMNNLKNEDTAMYYCVRDSHYYVSTYVGLAWFAYWGQ | |
| | GTLVTVSAASTKGPSVFPLAPSSKSTSGGTAALGCLVKDYFP | |
| | EPVTVSWNSGALTSGVHTFPAVLQSSGLYSLSSVVTVPSSSL | |
| | GTQTYICNVNHKPSNTKVDKKVEPKSCDKTHTCPPCPAPEL | |
| | LGGPSVFLFPPKPKDTLMISRTPEVTCVVVDVSHEDPEVKEN | |
| | WYVDGVEVHNAKTKPREEQYNSTYRVVSVLTVLHQDWLN | |
| | GKEYKCKVSNKALPAPIEKTISKAKGQPREPQVYTLPPSREE | |
| | MTKNQVSLTCLVKGFYPSDIAVEWESNGQPENNYKTTPPVL | |
| | DSDGSFFLYSKLTVDKSRWQQGNVFSCSVMHEALHNHYTQ | |
| | KSLSLSPG | |
| 13-E16 | QIVLTQSPAIMSASPGEKVTITCSASSSVSYMHWFQQKPGTS | 43 |
| chimera LC | PKLWIYSTSNLASGVPARFSGSGSGTSYFLTISRMEAEDAAT | |
| | YYCQQRSSYPYTFGGGTKLERKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| 19-007 VH | EVQLQQSVAELVRPGASVKLSCTASGFNIKNTQMHWVKQR | 44 |
| | PEQGLEWIGRIDPANGNTKYAPKFQGKATITGDTSSNTAYLQ | |
| | LSSLTSEDTAIYYCARFDYYYGSGDYAMDYWGQGTSVTVSS | |
| 19-007 VL | DVVMTQTPLTLSVTIGQPASISCKSSQSLLYSNGKTYLNWLL | 45 |
| | QRPGQSPKRLIYLVSKLDSGVPDRFTGSGSGTDFTLKISRVE | |
| | AEDLGVYYCVQGTRFPWTFGGGTNLEIK | |
| 19-007 HC | EVQLQQSVAELVRPGASVKLSCTASGFNIKNTQMHWVKQR | 46 |
| | PEQGLEWIGRIDPANGNTKYAPKFQGKATITGDTSSNTAYLQ | |
| | LSSLTSEDTAIYYCARFDYYYGSGDYAMDYWGQGTSVTVSS | |
| | TTTAPSVYPLVPGCSDTSGSSVTLGCLVKGYFPEPVTVKWN | |
| | YGALSSGVRTVSSVLQSGFYSLSSLVTVPSSTWPSQTVICNV | |
| | AHPASKTELIKRIEPRIPKPSTPPGSSCPPGNILGGPSVFIFPPK | |
| | PKDALMISLTPKVTCVVVDVSEDDPDVHVSWFVDNKEVHT | |
| | AWTQPREAQYNSTFRVVSALPIQHQDWMRGKEFKCKVNN | |
| | KALPAPIERTISKPKGRAQTPQVYTIPPPREQMSKKKVSLTCL | |
| | VTNFFSEAISVEWERNGELEQDYKNTPPILDSDGTYFLYSKL | |
| | TVDTDSWLQGEIFTCSVVHEALHNHHTQKNLSRSPELELNE | |
| | TCAEAQDGELDGLWTTITIFISLFLLSVCYSASVTLFKVKWIF | |
| | SSVVQVKQTAIPDYRNMIGQGA | |
| 19-007 LC | DVVMTQTPLTLSVTIGQPASISCKSSQSLLYSNGKTYLNWLL | 47 |
| | QRPGQSPKRLIYLVSKLDSGVPDRFTGSGSGTDFTLKISRVE | |
| | AEDLGVYYCVQGTRFPWTFGGGTNLEIKRADAAPTVSIFPP | |
| | SSEQLTSGGASVVCFLNNFYPKDINVKWKIDGSERQNGVLN | |
| | SWTDQDSKDSTYSMSSTLTLTKDEYERHNSYTCEATHKTST | |
| | SPIVKSFNRNEC | |
| 19-007 | GFNIKNTQMH | 48 |
| CDR-H1 | | |
| 19-007 | RIDPANGNTKYAP | 49 |
| CDR-H2 | | |
| 19-007 | ARFDYYYGSGDYAMDY | 50 |
| CDR-H3 | | |
| 19-007 | KSSQSLLYSNGKTYLN | 51 |
| CDR-L1 | | |
| 19-007 | LVSKLDS | 52 |
| CDR-L2 | | |
| 19-007 | VQGTRFPWT | 53 |
| CDR-L3 | | |
| 19-007 | EVQLQQSVAELVRPGASVKLSCTASGFNIKNTQMHWVKQR | 54 |
| chimera HC | PEQGLEWIGRIDPANGNTKYAPKFQGKATITGDTSSNTAYLQ | |
| | LSSLTSEDTAIYYCARFDYYYGSGDYAMDYWGQGTSVTVSS | |
| | ASTKGPSVFPLAPSSKSTSGGTAALGCLVKDYFPEPVTVSWN | |
| | SGALTSGVHTFPAVLQSSGLYSLSSVVTVPSSSLGTQTYICNV | |
| | NHKPSNTKVDKKVEPKSCDKTHTCPPCPAPELLGGPSVFLFP | |
| | PKPKDTLMISRTPEVTCVVVDVSHEDPEVKFNWYVDGVEV | |
| | HNAKTKPREEQYNSTYRVVSVLTVLHQDWLNGKEYKCKVS | |
| | NKALPAPIEKTISKAKGQPREPQVYTLPPSREEMTKNQVSLT | |
| | CLVKGFYPSDIAVEWESNGQPENNYKTTPPVLDSDGSFFLYS | |
| | KLTVDKSRWQQGNVFSCSVMHEALHNHYTQKSLSLSPG | |
| 19-007 | DVVMTQTPLTLSVTIGQPASISCKSSQSLLYSNGKTYLNWLL | 55 |
| chimera LC | QRPGQSPKRLIYLVSKLDSGVPDRFTGSGSGTDFTLKISRVE | |
| | AEDLGVYYCVQGTRFPWTFGGGTNLEIKRTVAAPSVFIFPPS | |
| | DEQLKSGTASVVCLLNNFYPREAKVQWKVDNALQSGNSQE | |
| | SVTEQDSKDSTYSLSSTLTLSKADYEKHKVYACEVTHQGLS | |
| | SPVTKSFNRGEC | |
| 1-K17 VH | QVQLQQPGAELVKPGASVKMSCKASGYTFTSYNMHWVKQ | 56 |
| | TPGQGLEWIGAIYPGNGDTSYNQKFKGKATLTADKSSSTAY | |
| | MHLSSLTSEDSAVYYCARKGGTPFAYWGQGTLLTVSA | |
| 1-K17 VL | DVVMTQTPLSLPVSLGDQASISCRSSQSLVHSNGNTYLYWY | 57 |
| | LQKPGQSPKLLIYKVSNRFSGVPDRESGSGSGTDFTLKISRVE | |
| | AEDLGVYFCSQSTHVPYTFGGGTKLEIK | |
| 1-K17 HC | QVQLQQPGAELVKPGASVKMSCKASGYTFTSYNMHWVKQ | 58 |
| | TPGQGLEWIGAIYPGNGDTSYNQKFKGKATLTADKSSSTAY | |
| | MHLSSLTSEDSAVYYCARKGGTPFAYWGQGTLLTVSAAKTT | |
| | PPSVYPLAPGSAAQTNSMVTLGCLVKGYFPEPVTVTWNSGS | |
| | LSSGVHTFPAVLESDLYTLSSSVTVPSSPRPSETVTCNVAHPA | |
| | SSTKVDKKIVPRDCGCKPCICTVPEVSSVFIFPPKPKDVLTITL | |
| | TPKVTCVVVDISKDDPEVQFSWFVDDVEVHTAQTQPREEQF | |
| | NSTFRSVSELPIMHQDWLNGKEFKCRVNSAAFPAPIEKTISK | |
| | TKGRPKAPQVYTIPPPKEQMAKDKVSLTCMITDFFPEDITVE | |
| | WQWNGQPAENYKNTQPIMNINGSYFVYSKLNVQKSNWEA | |
| | GNTFTCSVLHEGLHNHHTEKSLSHSPG | |
| 1-K17 LC | DVVMTQTPLSLPVSLGDQASISCRSSQSLVHSNGNTYLYWY | 59 |
| | LQKPGQSPKLLIYKVSNRFSGVPDRFSGSGSGTDFTLKISRVE | |
| | AEDLGVYFCSQSTHVPYTFGGGTKLEIKRADAAPTVSIFPPS | |
| | SEQLTSGGASVVCFLNNFYPKDINVKWKIDGSERQNGVLNS | |
| | WTDQDSKDSTYSMSSTLTLTKDEYERHNSYTCEATHKTSTS | |
| | PIVKSFNRNEC | |
| 1-K17, 1- | GYTFTSYNMH | 60 |
| K17.015 | | |
| CDR-H1, 1- | | |
| K17.044 CDR-H1 | | |
| 1-K17 | AIYPGNGDTSYNQ | 61 |
| CDR-H2 | | |
| 1-K17, 1- | ARKGGTPFAY | 62 |
| K17.015 | | |
| CDR-H1, 1- | | |
| K17.044 CDR-H3 | | |
| 1-K17 | RSSQSLVHSNGNTYLY | 63 |
| CDR-L1 | | |
| 1-K17, 1- | KVSNRFS | 64 |
| K17.015 | | |
| CDR-H1, 1- | | |
| K17.044 | | |
| CDR-L2 | | |
| 1-K17, 1- | SQSTHVPYT | 65 |
| K17.015 | | |
| CDR-H1, 1- | | |
| K17.044 CDR-L3 | | |
| 1-K17 | QVQLQQPGAELVKPGASVKMSCKASGYTFTSYNMHWVKQ | 66 |
| chimera HC | TPGQGLEWIGAIYPGNGDTSYNQKFKGKATLTADKSSSTAY | |
| | MHLSSLTSEDSAVYYCARKGGTPFAYWGQGTLLTVSAASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCDKTHTCPPCPAPELLGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 1-K17 | DVVMTQTPLSLPVSLGDQASISCRSSQSLVHSNGNTYLYWY | 67 |
| chimera LC | LQKPGQSPKLLIYKVSNRFSGVPDRFSGSGSGTDFTLKISRVE | |
| | AEDLGVYFCSQSTHVPYTFGGGTKLEIKRTVAAPSVFIFPPSD | |
| | EQLKSGTASVVCLLNNFYPREAKVQWKVDNALQSGNSQES | |
| | VTEQDSKDSTYSLSSTLTLSKADYEKHKVYACEVTHQGLSS | |
| | PVTKSFNRGEC | |
| 1-K17.015 | EVQLVQSGAEVKKPGASVKVSCKASGYTFTSYNMHWVRQ | 68 |
| VH | APGQRLEWIGAIYPGQGDTSYNQKFKGRATLTADKSASTAY | |
| | MELSSLRSEDTAVYYCARKGGTPFAYWGQGTLVTVSS | |
| 1-K17.015 | DVVMTQSPLSLPVTLGQPASISCRSSQSLVHSNAQTYLYWY | 69 |
| VL | QQRPGQSPRLLIYKVSNRFSGVPDRFSGSGSGTDFTLKISRVE | |
| | AEDVGVYFCSQSTHVPYTFGQGTKLEIK | |
| 1-K17.015 | EVQLVQSGAEVKKPGASVKVSCKASGYTFTSYNMHWVRQ | 70 |
| HC | APGQRLEWIGAIYPGQGDTSYNQKFKGRATLTADKSASTAY | |
| | MELSSLRSEDTAVYYCARKGGTPFAYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCDKTHTCPPCPAPELLGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 1-K17.015 | DVVMTQSPLSLPVTLGQPASISCRSSQSLVHSNAQTYLYWY | 71 |
| LC | QQRPGQSPRLLIYKVSNRFSGVPDRESGSGSGTDFTLKISRVE | |
| | AEDVGVYFCSQSTHVPYTFGQGTKLEIKRTVAAPSVFIFPPS | |
| | DEQLKSGTASVVCLLNNFYPREAKVQWKVDNALQSGNSQE | |
| | SVTEQDSKDSTYSLSSTLTLSKADYEKHKVYACEVTHQGLS | |
| | SPVTKSFNRGEC | |
| 1-K17.015 | AIYPGQGDTSYNQ | 72 |
| CDR-H2 | | |
| 1-K17.015 | RSSQSLVHSNAQTYLY | 73 |
| CDR-L1 | | |
| 1-K17.044 | EVQLVQSGAEVKKPGASVKVSCKASGYTFTSYNMHWVRQ | 74 |
| VH | APGQGLEWIGAIYPGAGDTSYNQKFKGRATLTADKSTSTAY | |
| | MELSSLRSEDTAVYYCARKGGTPFAYWGQGTLVTVSS | |
| 1-K17.044 | DVVMTQSPLSLPVTLGQPASISCRSSQSLVHSNAATYLYWYQ | 75 |
| VL | QRPGQSPRLLIYKVSNRFSGVPDRFSGSGSGTDFTLKISRVEA | |
| | EDVGVYFCSQSTHVPYTFGQGTKLEIK | |
| 1-K17.044 | EVQLVQSGAEVKKPGASVKVSCKASGYTFTSYNMHWVRQ | 76 |
| HC | APGQGLEWIGAIYPGAGDTSYNQKFKGRATLTADKSTSTAY | |
| | MELSSLRSEDTAVYYCARKGGTPFAYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCDKTHTCPPCPAPELLGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSREEMTKNQVSLTCLVK | |
| | GFYPSDIAVEWESNGQPENNYKTTPPVLDSDGSFFLYSKLTV | |
| | DKSRWQQGNVFSCSVMHEALHNHYTQKSLSLSPG | |
| 1-K17.044 | DVVMTQSPLSLPVTLGQPASISCRSSQSLVHSNAATYLYWYQ | 77 |
| LC | QRPGQSPRLLIYKVSNRFSGVPDRFSGSGSGTDFTLKISRVEA | |
| | EDVGVYFCSQSTHVPYTFGQGTKLEIKRTVAAPSVFIFPPSDE | |
| | QLKSGTASVVCLLNNFYPREAKVQWKVDNALQSGNSQESV | |
| | TEQDSKDSTYSLSSTLTLSKADYEKHKVYACEVTHQGLSSP | |
| | VTKSFNRGEC | |
| 1-K17.044 | AIYPGAGDTSYNQ | 78 |
| CDR-H2 | | |
| 1-K17.044 | RSSQSLVHSNAATYLY | 79 |
| CDR-L1 | | |
| 7-B16 VH | EVQLVESGGGLVQPKGSLKLSCAASGFTFNTYAMNWVRQA | 80 |
| | PGKGLEWVARIRSKSNNYATYYADSVKDRFTISRDDSQSML | |
| | YLQMNNLKTEDTAMYYCVRGLLRYRFFDVWGAGTTVTVS | |
| | S | |
| 7-B16 VL | DIVMTQAEPSVPVTPGESISISCRSSKSLLHSNGNTYLYWFLQ | 81 |
| | RPGQSPQLLIYRMSNLASGVPDRFSGSGSGTAFTLRISRVEAE | |
| | DVGVYYCMQHLEYPFTFGGGTKLEIK | |
| 7-B16 HC | EVQLVESGGGLVQPKGSLKLSCAASGFTFNTYAMNWVRQA | 82 |
| | PGKGLEWVARIRSKSNNYATYYADSVKDRFTISRDDSQSML | |
| | YLQMNNLKTEDTAMYYCVRGLLRYRFFDVWGAGTTVTVS | |
| | SAKTTPPSVYPLAPGSAAQTNSMVTLGCLVKGYFPEPVTVT | |
| | WNSGSLSSGVHTFPAVLESDLYTLSSSVTVPSSPRPSETVTCN | |
| | VAHPASSTKVDKKIVPRDCGCKPCICTVPEVSSVFIFPPKPKD | |
| | VLTITLTPKVTCVVVDISKDDPEVQFSWFVDDVEVHTAQTQ | |
| | PREEQFNSTFRSVSELPIMHQDWLNGKEFKCRVNSAAFPAPI | |
| | EKTISKTKGRPKAPQVYTIPPPKEQMAKDKVSLTCMITDFFP | |
| | EDITVEWQWNGQPAENYKNTQPIMNINGSYFVYSKLNVQK | |
| | SNWEAGNTFTCSVLHEGLHNHHTEKSLSHSPG | |
| 7-B16 LC | DIVMTQAEPSVPVTPGESISISCRSSKSLLHSNGNTYLYWFLQ | 83 |
| | RPGQSPQLLIYRMSNLASGVPDRFSGSGSGTAFTLRISRVEAE | |
| | DVGVYYCMQHLEYPFTFGGGTKLEIKRADAAPTVSIFPPSSE | |
| | QLTSGGASVVCFLNNFYPKDINVKWKIDGSERQNGVLNSW | |
| | TDQDSKDSTYSMSSTLTLTKDEYERHNSYTCEATHKTSTSPI | |
| | VKSFNRNEC | |
| 7-B16, 7- | GFTFNTYAMN | 84 |
| B16.001 | | |
| CDR-H1 | | |
| 7-B16, 7- | RIRSKSNNYATYYADSVKD | 85 |
| B16.001, 7- | | |
| B16.033 | | |
| CDR-H2 | | |
| 7-B16, 7- | VRGLLRYRFFDV | 86 |
| B16.001, 7- | | |
| B16.033 | | |
| CDR-H3 | | |
| 7-B16, 7- | RSSKSLLHSNGNTYLY | 87 |
| B16.001, 7- | | |
| B16.033 | | |
| CDR-L1 | | |
| 7-B16, 7- | RMSNLAS | 88 |
| B16.001, 7- | | |
| B16.033 | | |
| CDR-L2 | | |
| 7-B16, 7- | MQHLEYPFT | 89 |
| B16.001, 7- | | |
| B16.033 | | |
| CDR-L3 | | |
| 7-B16 | EVQLVESGGGLVQPKGSLKLSCAASGFTFNTYAMNWVRQ | 90 |
| chimera HC | PGKGLEWVARIRSKSNNYATYYADSVKDRFTISRDDSQSML | |
| | YLQMNNLKTEDTAMYYCVRGLLRYRFFDVWGAGTTVTVS | |
| | SASTKGPSVFPLAPSSKSTSGGTAALGCLVKDYFPEPVTVSW | |
| | NSGALTSGVHTFPAVLQSSGLYSLSSVVTVPSSSLGTQTYICN | |
| | VNHKPSNTKVDKKVEPKSCDKTHTCPPCPAPELLGGPSVFL | |
| | FPPKPKDTLMISRTPEVTCVVVDVSHEDPEVKFNWYVDGVE | |
| | VHNAKTKPREEQYNSTYRVVSVLTVLHQDWLNGKEYKCK | |
| | VSNKALPAPIEKTISKAKGQPREPQVYTLPPSREEMTKNQVS | |
| | LTCLVKGFYPSDIAVEWESNGQPENNYKTTPPVLDSDGSFFL | |
| | YSKLTVDKSRWQQGNVFSCSVMHEALHNHYTQKSLSLSPG | |
| 7-B16 | DIVMTQAEPSVPVTPGESISISCRSSKSLLHSNGNTYLYWFLQ | 91 |
| chimera LC | RPGQSPQLLIYRMSNLASGVPDRFSGSGSGTAFTLRISRVEAE | |
| | DVGVYYCMQHLEYPFTFGGGTKLEIKRTVAAPSVFIFPPSDE | |
| | QLKSGTASVVCLLNNFYPREAKVQWKVDNALQSGNSQESV | |
| | TEQDSKDSTYSLSSTLTLSKADYEKHKVYACEVTHQGLSSP | |
| | VTKSFNRGEC | |
| 7-B16.001 | EVQLVESGGGLVQPGGSLKLSCAASGFTFNTYAMNWVRQA | 92 |
| VH | SGKGLEWVGRIRSKSNNYATYYADSVKDRFTISRDDSKNTA | |
| | YLQMNSLKTEDTAVYYCVRGLLRYRFFDVWGQGTTVTVSS | |
| 7-B16.001 | DIVMTQSPLSLPVTPGEPASISCRSSKSLLHSNGNTYLYWFLQ | 93 |
| VL | KPGQSPQLLIYRMSNLASGVPDRFSGSGSGTDFTLKISRVEA | |
| | EDVGVYYCMQHLEYPFTFGGGTKVEIK | |
| 7-B16.001 | EVQLVESGGGLVQPGGSLKLSCAASGFTFNTYAMNWVRQA | 94 |
| HC | SGKGLEWVGRIRSKSNNYATYYADSVKDRFTISRDDSKNTA | |
| | YLQMNSLKTEDTAVYYCVRGLLRYRFFDVWGQGTTVTVSS | |
| | ASTKGPSVFPLAPSSKSTSGGTAALGCLVKDYFPEPVTVSWN | |
| | SGALTSGVHTFPAVLQSSGLYSLSSVVTVPSSSLGTQTYICNV | |
| | NHKPSNTKVDKKVEPKSCDKTHTCPPCPAPELLGGPSVFLFP | |
| | PKPKDTLMISRTPEVTCVVVDVSHEDPEVKFNWYVDGVEV | |
| | HNAKTKPREEQYNSTYRVVSVLTVLHQDWLNGKEYKCKVS | |
| | NKALPAPIEKTISKAKGQPREPQVYTLPPSREEMTKNQVSLT | |
| | CLVKGFYPSDIAVEWESNGQPENNYKTTPPVLDSDGSFFLYS | |
| | KLTVDKSRWQQGNVFSCSVMHEALHNHYTQKSLSLSPG | |
| 7-B16.001 | DIVMTQSPLSLPVTPGEPASISCRSSKSLLHSNGNTYLYWFLQ | 95 |
| LC | KPGQSPQLLIYRMSNLASGVPDRFSGSGSGTDFTLKISRVEA | |
| | EDVGVYYCMQHLEYPFTFGGGTKVEIKRTVAAPSVFIFPPSD | |
| | EQLKSGTASVVCLLNNFYPREAKVQWKVDNALQSGNSQES | |
| | VTEQDSKDSTYSLSSTLTLSKADYEKHKVYACEVTHQGLSS | |
| | PVTKSFNRGEC | |
| 7-B16.033 | EVQLVESGGGLVQPGRSLRLSCTASGFTFATYAMNWVRQAP | 96 |
| VH | GKGLEWVARIRSKSNNYATYYADSVKDRFTISRDDSKSILYL | |
| | QMNSLKTEDTAVYYCVRGLLRYRFFDVWGQGTTVTVSS | |
| 7-B16.033 | DIVMTQSPLSLPVTPGEPASISCRSSKSLLHSNGNTYLYWFLQ | 97 |
| VL | KPGQSPQLLIYRMSNLASGVPDRFSGSGSGTDFTLKISRVEA | |
| | EDVGVYYCMQHLEYPFTFGGGTKVEIK | |
| 7-B16.033 | EVQLVESGGGLVQPGRSLRLSCTASGFTFATYAMNWVRQAP | 98 |
| HC | GKGLEWVARIRSKSNNYATYYADSVKDRFTISRDDSKSILYL | |
| | QMNSLKTEDTAVYYCVRGLLRYRFFDVWGQGTTVTVSSAS | |
| | TKGPSVFPLAPSSKSTSGGTAALGCLVKDYFPEPVTVSWNSG | |
| | ALTSGVHTFPAVLQSSGLYSLSSVVTVPSSSLGTQTYICNVNH | |
| | KPSNTKVDKKVEPKSCDKTHTCPPCPAPELLGGPSVFLFPPK | |
| | PKDTLMISRTPEVTCVVVDVSHEDPEVKFNWYVDGVEVHN | |
| | AKTKPREEQYNSTYRVVSVLTVLHQDWLNGKEYKCKVSNK | |
| | ALPAPIEKTISKAKGQPREPQVYTLPPSREEMTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| 7-B16.033 | DIVMTQSPLSLPVTPGEPASISCRSSKSLLHSNGNTYLYWFLQ | 99 |
| LC | KPGQSPQLLIYRMSNLASGVPDRFSGSGSGTDFTLKISRVEA | |
| | EDVGVYYCMQHLEYPFTFGGGTKVEIKRTVAAPSVFIFPPSD | |
| | EQLKSGTASVVCLLNNFYPREAKVQWKVDNALQSGNSQES | |
| | VTEQDSKDSTYSLSSTLTLSKADYEKHKVYACEVTHQGLSS | |
| | PVTKSFNRGEC | |
| 7-B16.033 | GFTFATYAMN | 100 |
| CDR-H1 | | |
| Human | MDYTLDLSVTTVTDYYYPDIFSSPCDAELIQTNGKLLLAVFY | 101 |
| CCR8 | CLLFVFSLLGNSLVILVLVVCKKLRSITDVYLLNLALSDLLFV | |
| | FSFPFQTYYLLDQWVFGTVMCKVVSGFYYIGFYSSMFFITL | |
| | MSVDRYLAVVHAVYALKVRTIRMGTTLCLAVWLTAIMATIP | |
| | LLVFYQVASEDGVLQCYSFYNQQTLKWKIFTNFKMNILGLL | |
| | IPFTIFMFCYIKILHQLKRCQNHNKTKAIRLVLIVVIASLLFW | |
| | VPFNVVLFLTSLHSMHILDGCSISQQLTYATHVTEIISFTHCCV | |
| | NPVIYAFVGEKFKKHLSEIFQKSCSQIFNYLGRQMPRESCEK | |
| | SSSCQQHSSRSSSVDYIL | |
| Mouse | MDYTMEPNVTMTDYYPDFFTAPCDAEFLLRGSMLYLAILYC | 102 |
| CCR8 | VLFVLGLLGNSLVILVLVGCKKLRSITDIYLLNLAASDLLFVL | |
| | SIPFQTHNLLDQWVFGTAMCKVVSGLYYIGFFSSMFFITLMS | |
| | VDRYLAIVHAVYAIKVRTASVGTALSLTVWLAAVTATIPLMV | |
| | FYQVASEDGMLQCFQFYEEQSLRWKLFTHFEINALGLLLPFA | |
| | ILLFCYVRILQQLRGCLNHNRTRAIKLVLTVVIVSLLFWVPFN | |
| | VALFLTSLHDLHILDGCATRQRLALAIHVTEVISFTHCCVNPV | |
| | IYAFIGEKFKKHLMDVFQKSCSHIFLYLGRQMPVGALERQLS | |
| | SNQRSSHSSTLDDIL | |
| Cynomolgus | MDYTLDPSMTTMTDYYYPDSLSSPCDGELIQRNDKLLLAVF | 103 |
| monkey | YCLLFVFSLLGNSLVILVLVVCKKLRNITDIYLLNLALSDLLF | |
| CCR8 | VFSFPFQTYYQLDQWVFGTVMCKVVSGFYYIGFYSSMFFIT | |
| | LMSVDRYLAVVHAVYAIKVRTIRMGTTLSLVVWLTAIMATIP | |
| | LLVFYQVASEDGVLQCYSFYNQQTLKWKIFTNFEMNILGLLI | |
| | PFTIFMFCYIKILHQLKRCQNHNKTKAIRLVLIVVIASLLFWV | |
| | PFNVVLFLTSLHSMHILDGCSISQQLNYATHVTEIISFTHCCV | |
| | NPVIYAFVGEKFKKHLSEIFQKSCSHIFIYLGRQMPRESCEKS | |
| | SSCQQHSFRSSSIDYIL | |
| 7B16.002 | ARGLLRYRFFDV | 104 |
| CDRH3 | | |
| 7B16.003 | VAGLLRYRFFDV | 105 |
| CDRH3 | | |
| 7B16.004 | VRALLRYRFFDV | 106 |
| CDRH3 | | |
| 7B16.005 | VRGALRYRFFDV | 107 |
| CDRH3 | | |
| 7B16.006 | VRGLARYRFFDV | 108 |
| CDRH3 | | |
| 7B16.007 | VRGLLAYRFFDV | 109 |
| CDRH3 | | |
| 7B16.008 | VRGLLRARFFDV | 110 |
| CDRH3 | | |
| 7B16.009 | VRGLLRYAFFDV | 111 |
| CDRH3 | | |
| 7B16.010 | VRGLLRYRAFDV | 112 |
| CDRH3 | | |
| 7B16.011 | VRGLLRYRFADV | 113 |
| CDRH3 | | |
| 7B16.012 | VRGLLRYRFFAV | 114 |
| CDRH3 | | |
| 7B16.013 | VRGLLRYRFFDA | 115 |
| CDRH3 | | |
| 7B16- | ARGLLRARFFDV | 116 |
| H3C1.001 | | |
| CDRH3 | | |
| 7B16- | ARGLLRYRFFDA | 117 |
| H3C2.001 | | |
| CDRH3 | | |
| 7B16- | VRGLLRARFFDA | 118 |
| H3C3.001 | | |
| CDRH3 | | |
| 7B16- | ARGLLRARFFDA | 119 |
| H3C4.001 | | |
| CDRH3 | | |
| 7B16- | RSSQSLLHSNGYNYLD | 120 |
| L1.001 | | |
| CDRL1 | | |
| 7B16- | LGSNRAS | 121 |
| L2.001 | | |
| CDRL2 | | |
| 7B16- | MQALQTPFV | 122 |
| L3.001 | | |
| CDRL3 | | |
| 7B16- | GFTFSGSAMH | 123 |
| H1.001 | | |
| CDRH1 | | |
| 7B16- | RIRSKANSYATAYAASVKD | 124 |
| H2.001 | | |
| CDRH2 | | |
| 7B16- | TRYYYYYGMDV | 125 |
| H3.001 | | |
| CDRH3 | | |
| KRpep-2 | RRCPLYISYDPVCRR | 126 |
| KRpep-2d | RRRRCPLYISYDPVCRRRR | 127 |
| CCR8- | EEGKLVIWINGDKGYNGLAEVGKKFEKDTGIKVTVEHPDKL | 128 |
| NT.MBP | EEKFPQVAATGDGPDIIFWAHDRFGGYAQSGLLAEITPDKAF | |
| | QDKLYPFTWDAVRYNGKLIAYPIAVEALSLIYNKDLLPNPPK | |
| | TWEEIPALDKELKAKGKSALMFNLQEPYFTWPLIAADGGYA | |
| | FKYENGKYDIKDVGVDNAGAKAGLTFLIDLIKNKHMNADT | |
| | DYSIAEAAFNKGETAMTINGPWAWSNIDTSKVNYGVTVLPT | |
| | FKGQPSKPFVGVLSAGINAASPNKELAKEFLENYLLTDEGLE | |
| | AVNKDKPLGAVALKSYEEELAKDPRIAATMENAQKGEIMPN | |
| | IPQMSAFWYAVRTAVINAASGRQTVDEALKDAQTSGGGSGG | |
| | GSGGGSGGGSHHHHHHGGGSGGGGSGGENLYFQGMDYTL | |
| | DLSVTTVTDYYYPDIFSSPCDAELIQTNGK | |

1. A method of treating cancer in a subject comprising co-administering to the subject an effective amount of:
i) an anti-CCR8 antibody;
ii) a chemotherapeutic agent, and
iii) an anti-PD-1 antibody or anti-PD-L1 antibody
wherein the anti-CCR8 antibody has antibody-dependent cellular cytotoxicity (ADCC) activity and/or complement-dependent cytotoxicity (CDC) activity, and wherein the anti-CCR8 antibody is optionally a CCR8 neutralizing antibody.

2. The method of claim 1, wherein the chemotherapeutic agent is administered at a lower dose than in a standard of care chemotherapeutic regimen that does not comprise an anti-CCR8 antibody.

3.-5. (canceled)

6. The method of claim 1, wherein the chemotherapeutic agent is selected from the group consisting of a platinum complex, taxane, pemetrexed, gemcitabine, fluorouracil, irinotecan, etoposide, and doxorubicin.

7. (canceled)

8. The method of claim 6, wherein the platinum complex is selected from the group consisting of carboplatin, cisplatin, and oxaliplatin.

9. The method of claim 6, wherein the chemotherapeutic agent comprises a taxane.

10. The method of claim 9, wherein the taxane is docetaxel.

11. The method of claim 6, wherein the chemotherapeutic agent comprises gemcitabine.

12.-23. (canceled)

24. The method of claim 1, wherein the cancer is selected from the group consisting of breast cancer, pancreatic cancer, and lung cancer.

25.-28. (canceled)

29. The method of claim 24, wherein the cancer is lung cancer.

30. The method of claim 29, wherein the lung cancer is non-small cell lung cancer (NSCLC) or small cell lung cancer (SCLC).

31. (canceled)

32. The method of claim 30, wherein the lung cancer is NSCLC and the co-administered chemotherapeutic agent is selected from the group consisting of afatinib, albumin-bound paclitaxel, alectinib, cabozantinib, carboplatin, cisplatin, crizotinib, dabrafenib, docetaxel, erlotinib, etoposide, gemcitabine, paclitaxel, pemetrexed, vandetanib, vemurafenib, vinblastine, vinorelbine, and any combinations thereof.

33. The method of claim 30, wherein the lung cancer is SCLC and the co-administered chemotherapeutic agent is selected from the group consisting of 5-flourouracil, albumin bound paclitaxel, altretamine, anastrozole, capecitabine, carboplatin, cisplatin, cyclophosphamide, docetaxel, doxorubicin, etoposide, exemestane, gemcitabine, ifosfamide, irinotecan, letrozole, leuprolide acetate, liposomal doxorubicin, megestrol acetate, melphalan, olaparib, oxaliplatin, paclitaxel, pazopanib, pemetrexed, tamoxifen, topotecan, vinorelbine, and any combinations thereof.

34. The method of claim 1, where in the cancer is metastatic.

35.-37. (canceled)

38. The method of claim 1, wherein the subject is human.

39.-47. (canceled)

48. The method of claim 1, wherein the anti-CCR8 antibody comprises:
a. an HCDR1 comprising the amino acid sequence of SEQ ID NO: 12, an HCDR2 comprising the amino acid sequence of SEQ ID NO: 13, an HCDR3 comprising the amino acid sequence of SEQ ID NO: 14, an LCDR1 comprising the amino acid sequence of SEQ ID NO: 15, an LCDR2 comprising the amino acid sequence of SEQ ID NO: 16, and an LCDR3 comprising the amino acid sequence of SEQ ID NO: 17;
b. an HCDR1 comprising the amino acid sequence of SEQ ID NO: 24, an HCDR2 comprising the amino acid sequence of SEQ ID NO: 25, an HCDR3 comprising the amino acid sequence of SEQ ID NO: 26, an LCDR1 comprising the amino acid sequence of SEQ ID NO: 27, an LCDR2 comprising the amino acid sequence of SEQ ID NO: 28, and an LCDR3 comprising the amino acid sequence of SEQ ID NO: 29;
c. an HCDR1 comprising the amino acid sequence of SEQ ID NO: 36, an HCDR2 comprising the amino acid sequence of SEQ ID NO: 37, an HCDR3 comprising the amino acid sequence of SEQ ID NO: 38, an LCDR1 comprising the amino acid sequence of SEQ ID NO: 39, an LCDR2 comprising the amino acid sequence of SEQ ID NO: 40, and an LCDR3 comprising the amino acid sequence of SEQ ID NO: 41;
d. an HCDR1 comprising the amino acid sequence of SEQ ID NO: 48, an HCDR2 comprising the amino acid sequence of SEQ ID NO: 49, an HCDR3 comprising the amino acid sequence of SEQ ID NO: 50, an LCDR1 comprising the amino acid sequence of SEQ ID NO: 51, an LCDR2 comprising the amino acid sequence of SEQ ID NO: 52, and an LCDR3 comprising the amino acid sequence of SEQ ID NO: 53;
e. an HCDR1 comprising the amino acid sequence of SEQ ID NO: 60, an HCDR2 comprising the amino acid sequence of SEQ ID NO: 61, 72, or 78, an HCDR3 comprising the amino acid sequence of SEQ ID NO: 62, 73, or 79, an LCDR1 comprising the amino acid sequence of SEQ ID NO: 63, an LCDR2 comprising the amino acid sequence of SEQ ID NO: 64, and an LCDR3 comprising the amino acid sequence of SEQ ID NO: 65; or
f. an HCDR1 comprising the amino acid sequence of SEQ ID NO: 84 or 100, an HCDR2 comprising the amino acid sequence of SEQ ID NO: 85, an HCDR3 comprising the amino acid sequence of SEQ ID NO: 86, an LCDR1 comprising the amino acid sequence of SEQ ID NO: 87, an LCDR2 comprising the amino acid sequence of SEQ ID NO: 88, and an LCDR3 comprising the amino acid sequence of SEQ ID NO: 89.

49. The method of claim 1, wherein the anti-CCR8 antibody comprises:
a. a heavy chain variable region (VH) comprising an amino acid sequence that is at least 90%, 91%, 92%, 93%, 94%, 95%, 96%, 97%, 98%, or 99% identical to the amino acid sequence of SEQ ID NO: 68 or 74, and a light chain variable region (VL) comprising an amino acid sequence that is at least 90%, 91%, 92%, 93%, 94%, 95%, 96%, 97%, 98%, or 99% identical to the amino acid sequence of SEQ ID NO: 69 or 75; or
b. a heavy chain variable region (VH) comprising an amino acid sequence that is at least 90%, 91%, 92%, 93%, 94%, 95%, 96%, 97%, 98%, or 99% identical to the amino acid sequence of SEQ ID NO: 92 or 96, and a light chain variable region (VL) comprising an amino acid sequence that is at least 90%, 91%, 92%, 93%, 94%, 95%, 96%, 97%, 98%, or 99% identical to the amino acid sequence of SEQ ID NO: 93 or 97.

50.-54. (canceled)

55. The method of claim 1, wherein the anti-CCR8 antibody comprises:
a. a heavy chain (HC) comprising an amino acid sequence that is at least 90%, 91%, 92%, 93%, 94%, 95%, 96%, 97%, 98%, or 99% identical to the amino acid sequence of SEQ ID NO: 70 or 76, and a light chain (LC) comprising an amino acid sequence that is at least 90%, 91%, 92%, 93%, 94%, 95%, 96%, 97%, 98%, or 99% identical to the amino acid sequence of SEQ ID NO: 71 or 77; or
b. a heavy chain (HC) comprising an amino acid sequence that is at least 90%, 91%, 92%, 93%, 94%, 95%, 96%, 97%, 98%, or 99% identical to the amino acid sequence of SEQ ID NO: 94 or 98, and a light chain (LC) comprising an amino acid sequence that is at least 90%, 91%, 92%, 93%, 94%, 95%, 96%, 97%, 98%, or 99% identical to the amino acid sequence of SEQ ID NO: 95 or 99.

56. (canceled)

57. The method of claim 1, wherein the anti-CCR8 antibody is an afucosylated antibody.

58.-61. (canceled)

62. The method of claim 1, wherein the anti-CCR8 antibody is selected from the group consisting of BMS-986340 (Bristol Myers Squibb), LM-108 (LaNova Medicines), S-531011 (Shionogi), FPA157 (Five Prime, Amgen), IPG-7236 (Immunophage Biomedical), ICP-B05 (InnoCare Pharma Tech), SRF-114 (Surface Oncology), HBM1022 (Harbour BioMed), HFB1011 (HiFiBio), BAY-3375968 (Bayer), IO-1 (Oncurious), ZL-1218 (Zai Lab), GB2101 (Genor), and PSB-114 (Sound Biologics).

63. (canceled)

64. The method of claim 1, wherein the anti-PD-1 antibody or an anti-PD-L1 antibody is selected from the group consisting of pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, and zimberelimab.

65.-69. (canceled)
